(19) United States
(12) Patent Application Publication
(10) Pub. No.: US 2015/0218656 A1
(43) Pub. Date: Aug. 6, 2015

(54) METHODS FOR DETECTION AND DIAGNOSIS OF A LYMPHOID MALIGNANCY USING HIGH THROUGHPUT SEQUENCING

(71) Applicants: Adaptive Biotechnologies Corp., Seattle, WA (US); Brigham and Women's Hospital, Boston, MA (US)

(72) Inventors: Ilan Kirsch, Seattle, WA (US); Harlan Robins, Seattle, WA (US); Rachael Clark, Boston, MA (US); Thomas Kupper, Boston, MA (US)

(21) Appl. No.: 14/604,633

(22) Filed: Jan. 23, 2015

Related U.S. Application Data
(60) Provisional application No. 61/935,259, filed on Feb. 3, 2014.
(60) Provisional application No. 62/073,765, filed on Oct. 31, 2014.

(51) Int. Cl. C12Q 1/68

(52) U.S. Cl. CPC C12Q 1/6886; C12Q 2600/16; C12Q 2600/156

(57) ABSTRACT

Methods and compositions are provided for detection and diagnosis of a lymphoid malignancy using high throughput sequencing of rearranged T cell receptor DNA sequences.

### CROSS REFERENCE TO RELATED APPLICATIONS

This application claims the benefit of U.S. Provisional Application No. 61/935,259, filed Feb. 3, 2014, and U.S. Provisional Application No. 62/073,765, filed Oct. 31, 2014, each of which is herein incorporated by reference in its entirety.

### STATEMENT REGARDING FEDERALLY SPONSORED RESEARCH OR DEVELOPMENT

This invention was made with government support under R01 AR063962 and R01 AR056720 awarded by the National Institute of Arthritis and Musculoskeletal and Skin Diseases at the National Institutes of Health (NIH/NIAMS), R01 AI097128 awarded by the National Institute of Allergy and Infectious Diseases (NIAID), and the SPORE in Skin Cancer P50 CA9368305 awarded by the National Cancer Institute at the NIH (NIH/NCI). The government has certain rights in the invention.

### FIELD OF INVENTION

The invention relates to methods and compositions for, useful for detection and diagnosis of lymphoid neoplasms.

### BACKGROUND OF THE INVENTION

Lymphoid neoplasms are derived from a clonal expansion and proliferation of B- and T-lymphocytes. Suspicion of lymphoid leukemia and lymphoma in a subject may arise from clinical symptoms and/or an abnormal complete blood count. Immunophenotyping, morphologic evaluation, cytogenetics, and molecular analysis then form the foundation for a differential diagnosis. Conventional methods can include immunohistochemistry, flow cytometry, chromosome study, fluorescence in situ hybridization (FISH), polymerase chain reaction (PCR), and gene sequencing. However, current methods for detection and diagnosis of lymphoid neoplasms can be limited in sensitivity, accuracy or timeliness.

Cutaneous T-cell lymphomas (CTCL) are a category of lymphoid neoplasm and include a heterogeneous collection of non-Hodgkin's lymphomas derived from skin tropic T cells. CTCL encompasses skin limited variants, such as mycosis fungoides (MF) and leukemic forms of the disease, including Sézary syndrome (1). In MF, T cells are confined to fixed inflammatory skin lesions, the disease is often indolent, and approximately 80% of patients have a normal life expectancy (2). MF patients with progressive disease can develop skin tumors and lymph node involvement, but peripheral blood involvement is unusual. Patients with leukemic CTCL (L-CTCL, including Sézary syndrome) present most commonly with diffuse skin erythema, lymphadenopathy and malignant T cells accumulate in the blood, skin and lymph nodes. L-CTCL is often refractory to therapy and survival is three years with death occurring most commonly from infection. Hematopoietic stem cell transplantation is the only potentially definitive cure for both advanced MF and L-CTCL (3).

Early diagnosis of CTCL can be challenging, particularly in MF. CTCL originates in solid tissue, and the skin lesions of MF can clinically and histologically resemble those of benign inflammatory disorders including psoriasis and atopic dermatitis. CTCL can be difficult to diagnose because the lymphoid cells make up a small fraction of the total tissue cells, the cells are embedded in the solid tissue, and the histopathologic appearance can resemble both malignant and benign conditions (Pimpinalli N, Olsen E A, Santucci M, Vonderheid E, Haeffner A C, Stevens S, et al. Defining Early Mycosis Fungoides, J Am Acad Dermatol 53: 1053-1063 (2005) Guitart J, and Magro C. Cutaneous T-cell Lymphoid Dyscrasia: A Unifying Term for Idiopathic Chronic Dermatoses with Persistent T-cell Clones, Arch Dermatol 143: 921-932 (2007)).

Conventionally, the diagnosis of CTCL is based on a combination of findings including the clinical presentation, suggestive histopathology and identification of a clonal T-cell population in blood or skin lesions.

The most commonly used clinical test, multiplex/heteroduplex PCR amplification of the TCR Vγ chain followed by GeneScan capillary electrophoresis analysis, detects clones in only a subset of patients with CTCL (5, 6). As a result, the diagnosis of MF is commonly delayed and is made on average six years after the first development of skin lesions (7).

High-throughput sequencing (HTS) is an emerging technology that can provide insight into the complexity of the adaptive immune response through the analysis of lymphoid receptor gene rearrangement. Robins et al., Blood 114, 4099-4107 (2009). Studies using this technology have challenged understanding in the art of the extent of lymphocyte diversity occurring within, and shared by, individuals (Robins et al., Blood 114, 4099-4107 (2009), Robins et al., Sci Transl Med 2, 47ra64 (2010)), and have provided mechanistic insight into the early molecular genetic events critical for the T-cell lineage maturation (Sherwood et al., Sci Transl Med 3, 90ra61 (2011)). Recently, high-throughput sequencing of lymphoid cell adaptive immune receptor genes has been used to monitor lymphocyte diversity after adoptive immunotherapy with chimeric antigen receptor-modified T cells for the treatment of chemotherapy-refractory chronic lymphocytic leukemia (Kalos et al., Sci Transl Med 3, 95ra73 (2011).). Separately, high-throughput sequencing of lymphoid cell adaptive immune receptor genes has been used for monitoring disease in B lymphoproliferative disorders (Boyd et al., Sci Transl Med 1, 12ra23 (2009)). HTS has exhibited the ability to identify rare T cell clones (one T cell in 100,000) with high accuracy and reproducibility (Robins, Desmarais, Matthis, Livingston, Andriesen, Reijonen, Carlson, Nepom, Yee, Cerosaletti, Ultra-sensitive detection of rare T cell clones. J Immunol Methods doi:10.1016/j.jim.2011.09.001 (Sep. 10, 2011 Epub ahead of print PMID 21945395)). Another study demonstrated the evaluation of minimal residual disease in precursor acute T lymphoblastic leukemias (Wu, D, et. al., Sci. Transl. Med. 2012).

Thus, there is a need for improved sensitivity and specificity in the diagnosis of lymphoid malignancies that is reflected in the heterogeneity and relative frequencies of occurrence of particular unique adaptive immune receptors, and in the ability to detect malignant lymphoid cells. In particular, methods are needed for accurately and reliably detecting lymphoid neoplasms that are difficult to diagnose, such as, for example, discriminating between CTCL and benign inflammatory skin diseases, in a timely manner. Methods are needed to both facilitate initial diagnosis of lymphoid malignancies (T- and B-cell malignancies) and to discriminate malignant recurrences from benign inflammatory reactions. The presently described embodiments address these needs and provide other related advantages.

### SUMMARY OF THE INVENTION

Methods of the invention include a method for determining a threshold for detecting lymphoid malignancies in a human subject, comprising for a plurality of samples each comprising genomic DNA and obtained from a sample from a human subject suspected of having a lymphoid malignancy, generating a profile of rearranged T-cell receptor (TCR) complementarity determining region-3 (CDR3) sequences in the sample, the profile comprising a frequency of occurrence for each unique TCR CDR3 rearranged sequence.

In some embodiments, determining the T cell clone with the highest frequency of occurrence comprises determining the frequency of the most frequent T cell clone as a fraction of a total number of nucleated cells in the sample.

In other embodiments, determining the T cell clone with the highest frequency of occurrence in the sample comprises determining a percentage.

In some embodiments, the threshold is a proportion of 1 most frequent T cell clone in 500 total nucleated cells. In other embodiments, the threshold is a proportion of 1 most frequent T cell clone in 1000 total nucleated cells.

In one embodiment, the threshold is equal to or greater than a proportion of 1 in 500 total nucleated cells. In another embodiment, the threshold is equal to or greater than a proportion of 1 in 1000 total nucleated cells. In yet another embodiment, the threshold is used for detecting a presence of CTCL in the subject.

In certain embodiments, the threshold is used for detecting a recurrence of a lymphoid malignancy in the subject.

In certain embodiments, a frequency of occurrence for a most frequent T cell clone that is above the threshold is indicative of a lymphoid malignancy. In another embodiment, a frequency of occurrence for the most frequent T cell clone that is statistically significantly higher than the threshold is indicative of a lymphoid malignancy. In yet another embodiment, a frequency of occurrence for the most frequent T cell clone that is below the threshold is indicative of a non-malignant condition.

In some embodiments, the T-cell receptor (TCR) is encoded by a TCR 0 gene. In another embodiment, the T-cell receptor (TCR) is encoded by a TCR γ gene.

In some embodiments, identifying the most frequent T cell clone comprises combining the frequencies of occurrence of the top two most frequent TCRγ gene sequences in the sample.

In certain embodiments, generating a profile of rearranged T-cell receptor (TCR) complementarity determining region-3 (CDR3) sequences in the sample comprises: amplifying the rearranged T-cell receptor (TCR) complementarity determining region-3 (CDR3) sequences in a single multiplex PCR using a plurality of V-segment primers and a plurality of J-segment primers to produce a plurality of amplicons representing the diversity of TCR genes in the sample; and sequencing the plurality of amplicons to produce a plurality of sequence reads.

Methods of the invention include a method for diagnosing cutaneous T cell lymphoma (CTCL) in a human subject, comprising: obtaining a genomic DNA sample from the human subject; generating a profile of rearranged T-cell receptor (TCR) complementarity determining region-3 (CDR3) sequences, the profile comprising a frequency of each unique TCR CDR3 rearranged sequence; identifying a T cell clone with the highest frequency of occurrence in a total number of nucleated cells in the sample; and determining whether the T cell clone with the highest frequency of occurrence has a frequency of occurrence that is above or below a predetermined threshold for malignancy, wherein a frequency of occurrence above the predetermined threshold is consistent with a lymphoid malignancy in the subject. In some embodiments, the method includes diagnosing the subject with a lymphoid malignancy. In one embodiment, diagnosis is at an early stage for the particular malignancy. In certain embodiments, the method includes detecting a lymphoid malignancy in a subject in whom a lymphoid malignancy was not identified using another detection method. The predetermined threshold for malignancy is a threshold of 1 in 1000 total nucleated cells.

In some embodiments, the predetermined threshold for malignancy is equal to or greater than a threshold of 1 in 1000 total nucleated cells. In certain embodiments, the method includes determining that the most frequent T cell clone has a frequency of occurrence that is at least one standard deviation above or below the predetermined threshold. In one embodiment, the method includes determining that the most frequent T cell clone has a frequency of occurrence that is statistically significantly different at p<0.05 from the predetermined threshold. In another embodiment, the method includes determining that the most frequent T cell clone has a frequency of occurrence that is statistically significantly different at p<0.01 from the predetermined threshold. In yet another embodiment, the method includes determining that the most frequent T cell clone has a frequency of occurrence that is statistically significantly different at p<0.001 from the predetermined threshold.

In one embodiment, the sample is obtained from a blood sample.

In another aspect, generating a profile of rearranged T-cell receptor (TCR) complementarity determining region-3 (CDR3) sequences comprises amplifying the rearranged T-cell receptor (TCR) complementarity determining region-3 (CDR3) sequences in a single multiplex PCR using a plurality of V-segment primers and a plurality of J-segment primers to produce a plurality of amplicons representing the diversity of TCR genes in the sample; and sequencing the plurality of amplicons to produce a plurality of sequence reads.

In other aspects, the method includes correcting for amplification bias in the plurality of V-segment primers and plurality of J-segment primers.

The invention includes a kit for diagnosing a lymphoid malignancy in a human subject, comprising compositions for amplifying genomic DNA obtained from a sample from the human subject in a single multiplex PCR; and instructions for amplification of the genomic DNA and high throughput sequencing and instructions for determining whether a top T cell clone in the sample has a frequency of occurrence that is above or below a predetermined threshold for malignancy, wherein a frequency of occurrence above the predetermined threshold is consistent with a lymphoid malignancy in the subject.

In one embodiment, the lymphoid malignancy is selected from: acute T-cell lymphoblastic leukemia (T-ALL), acute B-cell lymphoblastic leukemia (B-ALL), multiple myeloma, plasmacytoma, macroglobulinemia, chronic lymphocytic leukemia (CLL), acute lymphoblastic leukemia (ALL), multiple myeloma, plasmacytoma, macroglobulinemia, Hodgkins lymphoma, non-Hodgkins lymphoma, cutaneous T-cell lymphoma (CTCL), mantle cell lymphoma, peripheral T-cell lymphoma, hairy cell leukemia, T prolymphocytic lymphoma, angioimmunoblastic T-cell lymphoma, T lymphoblastic leukemia/lymphoma, peripheral T-cell lymphoma, adult T cell leukemia/lymphoma, mycosis fungoides, Sezary syndrome, T lymphoblastic leukemia, myeloproliferative neoplasm, and myelodysplastic syndrome. In one embodiment, the lymphoid malignancy is CTCL.

According to certain embodiments of the invention described herein, there is provided a method for determining a malignancy score for unique rearranged amplicon sequences of a sample to determine whether a sample comprises malignant lymphoid cells. First, rearranged amplicon sequence information is received from a sample of solid tissue from a subject. The rearranged amplicon sequence information includes unique rearranged amplicon sequences, each of which encodes a gene segment of an adaptive immune receptor (AIR) polypeptide. The total number of observed rearranged amplicon sequences in the sample is determined, as is the total number of unique rearranged amplicon sequences in the sample. A frequency of occurrence of each unique rearranged amplicon sequence is determined. In one embodiment, one or more of the unique rearranged amplicon sequences having a frequency of occurrence in the sample greater than a threshold frequency of occurrence is determined. In another embodiment, a malignancy score is determined for the one or more of the unique rearranged amplicon sequences, the malignancy score based on comparing the frequency of occurrence of the one or more of the unique rearranged amplicon sequences with the threshold frequency of occurrence. A malignancy score that is statistically significantly different from a reference score indicates that the sample of solid tissue includes malignant lymphoid cells, and a malignancy score that is not statistically significantly different from a reference score indicates that the sample of solid tissue does not include malignant lymphoid cells.

In an embodiment, the threshold frequency of occurrence is based on an average frequency of occurrence of one or more of the unique rearranged amplicon sequences in non-malignant cells. In a further embodiment, the threshold frequency of occurrence is further based on a standard deviation of the average frequency of occurrence of one or more of the unique rearranged amplicon sequences in non-malignant cells. In another embodiment, determining the malignancy score includes dividing the frequency of occurrence of the one or more of the unique rearranged amplicon sequences by the threshold frequency of occurrence. In yet another embodiment, two of the unique rearranged amplicon sequences are determined having frequencies of occurrence in the sample at frequencies greater than the threshold frequency of occurrence. In a further embodiment, determining the malignancy score includes summing the frequencies of occurrence of the two unique rearranged amplicons sequences, and dividing the sum by the total number of genomes from the sample. In another embodiment, the frequency of occurrence of each unique rearranged amplicon sequence is calculated as a percentage of the total number of observed rearranged amplicon sequences. In another embodiment, the frequency of occurrence of each unique rearranged amplicon sequence is calculated as a percentage of a total number of genomes from the sample.

In one embodiment, the rearranged amplicon sequence information is obtained by amplifying a multiplex polymerase chain reaction (PCR) from the sample a plurality of gene segments of the AIR polypeptide, each gene segment including a variable (V)-region and a joining (J)-region, using: V-segment oligonucleotide primers and J-segment oligonucleotide primers. Each V-segment oligonucleotide primer is capable of hybridizing to one or more V-region polynucleotides of the gene segments, and each J-segment oligonucleotide primer is capable of hybridizing to one or more J-region polynucleotides of the gene segments. The V-segment oligonucleotide primers and J-segment oligonucleotide primers promote amplification of the plurality of gene segments of an AIR polypeptide to produce amplicons. The amplicons are sequenced to determine a nucleotide sequence for each of the amplicons.

In a further embodiment, the V-segment oligonucleotide primers are each capable of specifically hybridizing to at least one polynucleotide encoding a mammalian AIR V-region polypeptide. Each AIR V-segment oligonucleotide primer includes a nucleotide sequence of at least 15 contiguous nucleotides that is complementary to at least one functional AIR V-encoding gene segment, and the plurality of AIR V-segment oligonucleotide primers specifically hybridize to substantially all functional AIR V-encoding gene segments that are present in the sample. Furthermore, the J-segment oligonucleotide primers are each capable of specifically hybridizing to at least one polynucleotide encoding a mammalian AIR J-region polypeptide. Each AIR J-segment oligonucleotide primer includes a nucleotide sequence of at least 15 contiguous nucleotides that is complementary to at least one functional AIR J-encoding gene segment, and the plurality of AIR J-segment oligonucleotide primers specifically hybridize to substantially all functional AIR J-encoding gene segments that are present in the sample. Also, the AIR V-segment oligonucleotide primers and the AIR J-segment oligonucleotide primers are capable of promoting amplification in the multiplex PCR of substantially all rearranged AIR CDR3-encoding regions in the sample to produce a plurality of amplified rearranged amplicon sequences sufficient to quantify the full diversity of the AIR CDR3-encoding region in the sample.

In another embodiment, each of the V-segment oligonucleotide primers and each of the J-segment oligonucleotide primers includes a nucleotide sequence of at least 15 contiguous nucleotides. In other embodiments, each amplified rearranged nucleic acid molecule is less than 1500, 1000, 600, 500, 400, 300, 200, or 100 nucleotides in length. In an embodiment, each amplified rearranged nucleic acid molecule is between 50-600 nucleotides in length.

In a separate embodiment, the unique rearranged amplicon sequences include genomic DNA sequences. In another embodiment, a treatment is provided for the subject based on the determination that the sample comprises malignant lymphoid cells. In another embodiment, the AIR polypeptide is a mammalian AIR polypeptide and is selected from a T-cell receptor-gamma (TCRG) polypeptide, a T-cell receptor-beta (TCRB) polypeptide, a T-cell receptor-alpha (TCRA) polypeptide, a T-cell receptor-delta (TCRD) polypeptide, an immunoglobulin heavy-chain (IGH) polypeptide, and an immunoglobulin light-chain (IGL) polypeptide. In a further embodiment, the IgH polypeptide is selected from an IgM polypeptide, an IgA polypeptide, an IgG polypeptide, an IgD polypeptide and an IgE polypeptide. In another embodiment, the IgL polypeptide is selected from an IGL-lambda polypeptide and an IGL-kappa polypeptide. In other embodiments, the mammalian AIR polypeptide is a human AIR polypeptide, a non-human primate AIR polypeptide, a rodent AIR polypeptide, a canine AIR polypeptide, a feline AIR polypeptide or an ungulate AIR polypeptide.

In one embodiment, the lymphoid malignancy is selected from: acute T-cell lymphoblastic leukemia (T-ALL), acute B-cell lymphoblastic leukemia (B-ALL), multiple myeloma, plasmacytoma, macroglobulinemia, chronic lymphocytic leukemia (CLL), acute lymphoblastic leukemia (ALL), multiple myeloma, plasmacytoma, macroglobulinemia, Hodgkins lymphoma, non-Hodgkins lymphoma, cutaneous T-cell lymphoma (CTCL), mantle cell lymphoma, peripheral T-cell lymphoma, hairy cell leukemia, T prolymphocytic lymphoma, angioimmunoblastic T-cell lymphoma, T lymphoblastic leukemia/lymphoma, peripheral T-cell lymphoma, adult T cell leukemia/lymphoma, mycosis fungoides, Sezary syndrome, T lymphoblastic leukemia, myeloproliferative neoplasm, and myelodysplastic syndrome. In one embodiment, the lymphoid malignancy is CTCL.

Methods of the invention include a method for determining a threshold for detecting cutaneous T cell lymphoma (CTCL) in a human subject, comprising for a plurality of samples each comprising genomic DNA and obtained from a skin biopsy of a human subject, generating a profile of rearranged T-cell receptor (TCR) complementarity determining region-3 (CDR3) sequences in the sample, the profile comprising a frequency of occurrence for each unique TCR CDR3 rearranged sequence. The method includes determining a T cell clone with the highest frequency of occurrence in the sample; identifying the T cell clone with the highest frequency in a sample from a subject with a non-malignant skin condition or a subject previously diagnosed with CTCL; comparing the frequencies of occurrence of the T cell clones with the highest frequency from subjects with a non-malignant skin condition with the frequencies of occurrence of T cell clones from subjects previously diagnosed with CTCL; and determining a threshold for detecting CTCL in a sample based on the comparison.

In some embodiments, determining the T cell clone with the highest frequency of occurrence comprises determining the frequency of the most frequent T cell clone as a fraction of a total number of nucleated cells in the sample.

In other embodiments, determining the T cell clone with the highest frequency of occurrence in the sample comprises determining a percentage.

In some embodiments, the threshold is a proportion of 1 most frequent T cell clone in 500 total nucleated cells. In other embodiments, the threshold is a proportion of 1 most frequent T cell clone in 1000 total nucleated cells.

In one embodiment, the threshold is equal to or greater than a proportion of 1 in 500 total nucleated cells. In another embodiment, the threshold is equal to or greater than a proportion of 1 in 1000 total nucleated cells. In yet another embodiment, the threshold is used for detecting a presence of CTCL in the subject.

In certain embodiments, the threshold is used for detecting a recurrence of CTCL in the subject.

In certain embodiments, a frequency of occurrence for a most frequent T cell clone that is above the threshold is indicative of CTCL. In another embodiment, a frequency of occurrence for the most frequent T cell clone that is statistically significantly higher than the threshold is indicative of CTCL. In yet another embodiment, a frequency of occurrence for the most frequent T cell clone that is below the threshold is indicative of a non-malignant skin condition.

In one embodiment, a non-malignant skin condition is a benign inflammatory skin disease. In another embodiment, the non-malignant skin condition is selected from the group consisting of: psoriasis, eczematous dermatitis, and normal healthy skin.

In some embodiments, the T-cell receptor (TCR) is encoded by a TCR 0 gene. In another embodiment, the T-cell receptor (TCR) is encoded by a TCR γ gene.

In some embodiments, identifying the most frequent T cell clone comprises combining the frequencies of occurrence of the top two most frequent TCRγ gene sequences in the sample.

In certain embodiments, generating a profile of rearranged T-cell receptor (TCR) complementarity determining region-3 (CDR3) sequences in the sample comprises: amplifying the rearranged T-cell receptor (TCR) complementarity determining region-3 (CDR3) sequences in a single multiplex PCR using a plurality of V-segment primers and a plurality of J-segment primers to produce a plurality of amplicons representing the diversity of TCR genes in the sample; and sequencing the plurality of amplicons to produce a plurality of sequence reads.

Methods of the invention include a method for diagnosing cutaneous T cell lymphoma (CTCL) in a human subject, comprising: obtaining a genomic DNA sample from the human subject; generating a profile of rearranged T-cell receptor (TCR) complementarity determining region-3 (CDR3) sequences, the profile comprising a frequency of each unique TCR CDR3 rearranged sequence; identifying a T cell clone with the highest frequency of occurrence in a total number of nucleated cells in the sample; and determining whether the T cell clone with the highest frequency of occurrence has a frequency of occurrence that is above or below a predetermined threshold for malignancy, wherein a frequency of occurrence above the predetermined threshold is consistent with CTCL in the subject. In some embodiments, the method includes diagnosing the subject with CTCL. In one embodiment, diagnosis is at an early stage of CTCL. In certain embodiments, the method includes detecting CTCL in a subject in whom CTCL was not identified using another detection method. The predetermined threshold for malignancy is a threshold of 1 in 1000 total nucleated cells.

In some embodiments, the predetermined threshold for malignancy is equal to or greater than a threshold of 1 in 1000 total nucleated cells. In certain embodiments, the method includes determining that the most frequent T cell clone has a frequency of occurrence that is at least one standard deviation above or below the predetermined threshold. In one embodiment, the method includes determining that the most frequent T cell clone has a frequency of occurrence that is statistically significantly different at p<0.05 from the predetermined threshold. In another embodiment, the method includes determining that the most frequent T cell clone has a frequency of occurrence that is statistically significantly different at p<0.01 from the predetermined threshold. In yet another embodiment, the method includes determining that the most frequent T cell clone has a frequency of occurrence that is statistically significantly different at p<0.001 from the predetermined threshold.

In one embodiment, the sample is obtained from a skin biopsy. In another embodiment, the sample is obtained from a blood sample.

In one aspect, the method includes comparing a frequency of occurrence of the most frequent T cell clone in a skin biopsy and a frequency of occurrence of the most frequent T cell clone in a blood sample.

In another aspect, generating a profile of rearranged T-cell receptor (TCR) complementarity determining region-3 (CDR3) sequences comprises amplifying the rearranged T-cell receptor (TCR) complementarity determining region-3 (CDR3) sequences in a single multiplex PCR using a plurality of V-segment primers and a plurality of J-segment primers to produce a plurality of amplicons representing the diversity of TCR genes in the sample; and sequencing the plurality of amplicons to produce a plurality of sequence reads.

In other aspects, the method includes correcting for amplification bias in the plurality of V-segment primers and plurality of J-segment primers.

The invention includes a kit for diagnosing cutaneous T cell lymphoma (CTCL) in a human subject, comprising compositions for amplifying genomic DNA obtained from a sample from the human subject in a single multiplex PCR; and instructions for amplification of the genomic DNA and high throughput sequencing and instructions for determining whether a top T cell clone in the sample has a frequency of occurrence that is above or below a predetermined threshold for malignancy, wherein a frequency of occurrence above the predetermined threshold is consistent with CTCL in the subject.

Methods of the invention include determining a threshold for detecting a lymphoid malignancy in a human subject, comprising: generating a profile of rearranged B-cell receptor (BCR) complementarity determining region-3 (CDR3) sequences from genomic DNA obtained from a sample from the human subject, the profile comprising a frequency of occurrence for each unique BCR CDR3 rearranged sequence; determining a B cell clone with the highest frequency of occurrence in the sample; identifying the B cell clone with the highest frequency in a sample from a subject with a non-malignant condition or a subject previously diagnosed with a lymphoid malignancy; comparing the frequencies of occurrence of the B cell clones with the highest frequency from subjects with a non-malignant condition with the frequencies of occurrence of B cell clones from subjects previously diagnosed with a lymphoid malignancy; and determining a threshold for detecting a lymphoid malignancy in a sample based on the comparison.

In one embodiment, determining the B cell clone with the highest frequency of occurrence comprises determining the frequency of the most frequent B cell clone as a fraction of a total number of nucleated cells in the sample.

In another embodiment, determining the B cell clone with the highest frequency of occurrence in the sample comprises determining a percentage.

In some embodiments, the threshold is a proportion of 1 most frequent B cell clone in 1000 total nucleated cells. In another embodiment, the threshold is equal to or greater than a proportion of 1 in 1000 total nucleated cells.

### BRIEF DESCRIPTION OF THE SEVERAL VIEWS OF THE DRAWINGS

These and other features, aspects, and advantages of the present invention will become better understood with regard to the following description, and accompanying drawings, where:

Figure (FIG.) 1 is a box and whiskers plot showing the distribution of the most abundant TCR clone as a percent of TCR clones in control samples by tissue type, according to an embodiment of the invention.

FIG. 2 is a graph showing the identification of diagnostic clones in various sample based on the frequency of occurrence of the unique rearranged amplicon sequence in the total number of observed rearranged amplicon sequences expressed as a percentage, according to an embodiment of the invention.

FIG. 3 illustrates the top TCRB clone (%) determined from biopsy samples taken from a patient at four different time points over six years, according to an embodiment of the invention.

FIG. 4 illustrates various methods of tracking residual disease, and shows whether a particular method positively or negatively identified residual disease in a sample, according to an embodiment of the invention.

FIG. 5 illustrates the summed frequencies of occurrence of two unique rearranged amplicon sequences divided by the total number of nucleated cells, from various samples, according to an embodiment of the invention.

FIG. 6 illustrates a box and whiskers plot comparing samples diagnosed with CTCL versus other skin lesion categories, and illustrating the distribution of the two unique rearranged amplicon sequences with the highest frequencies of occurrence over the total nucleated cell population, according to an embodiment of the invention.

FIG. 7A shows increasing clonality of lesional skin T cells with advanced stages of CTCL. The mean and SEM of clonality scores are shown of 8 patients with Stage IA, 18 patients with stage IB, 5 patients with stage IIA, 7 patients with stage IIB, 2 patients with stage III, and 5 patients with stage IV disease. High throughput TCRβ CDR3 region sequencing was used to identify expanded T cell clones and to discriminate CTCL from benign inflammatory skin disorders. DNA was isolated from the lesional skin of patients with CTCL, psoriasis, eczematous dermatitis (ED) and from the skin of healthy individuals (Nml skin) and subjected to high throughput deep TCRVβ sequencing.

FIG. 7B shows a 3-D histograph of TCR sequencing results that show V gene vs. J gene usages of T cells from a lesional skin sample. Expanded populations of clonal malignant T cells in CTCL skin lesions are shown. The tallest (green) peak includes the clonal malignant T cell population, as well as other benign T cells that share the same V and J gene usage.

FIG. 7C shows percent of clones for the top 10 T cell clones in a subject. The individual top T cell clone sequence is shown (1), with detailed information on the CDR3 AA sequence and V and J gene usage. The nine most frequent benign infiltrating T cell sequences are also shown (2-9). In this patient, the malignant T cell clone made up 10.3% of the total T cell population in lesional skin.

FIG. 7D shows the most frequent T cell clone expressed as a percentage of total T cells for individual samples. The most frequent T cell clone expressed as a percentage of total T cells did not completely discriminate CTCL from patients with benign inflammatory skin disease.

FIG. 7E shows the most frequent T cell sequences expressed as a percentage of total T cell is shown for aggregate data for subjects with CTCL, psoriasis, eczematous dermatitis (ED) and from the skin of healthy individuals (Nml skin).

FIG. 7F shows the most frequent T cell clone expressed as the fraction of total nucleated cells for individual samples from subjects with CTCL, psoriasis, eczematous dermatitis (ED) and from the skin of healthy individuals (Nml skin) The most frequent T cell clone expressed as the fraction of total nucleated cells successfully discriminates CTCL from benign inflammatory skin diseases.

FIG. 7G shows the most frequent T cell sequence expressed as a fraction of total nucleated cells is shown for aggregate data (G) of samples from subjects with CTCL, psoriasis, eczematous dermatitis (ED) and from the skin of healthy individuals (Nml skin) This analysis allowed discrimination of CTCL from benign inflammatory skin diseases and healthy skin (p<0.001 for all groups). *p<0.05, **p<0.01,***p<0.001.

FIG. 8A shows results of high throughput sequencing of both the TCRγ and TCRβ CDR3 regions obtained from skin samples. The top TCRβ sequence and the sum of the top two TCRγ sequences divided by two were expressed as the fraction of total nucleated cells, and the results from TCRγ and TCRβ sequencing are compared. In general, there was close concordance between TCRγ and TCRβ sequencing results. The exception was one patient with a known γδ T cell malignancy, in whom TCRγ sequencing identified a malignant clone but TCRβ did not, consistent with the known lack of TCR Vβ gene rearrangement in γδT cells. High throughput sequencing of the TCR γ CDR3 genes also discriminates CTCL from benign inflammatory skin diseases.

FIG. 8B shows the TCR γ fraction of nucleated cells (sum of the top two TCR γ clones divided by two) for individual samples expressed as a fraction of total nucleated cells discriminates CTCL from benign inflammatory skin diseases.

FIG. 8C shows the sum of the top two TCR γ clones divided by two for an aggregate of samples expressed as a fraction of total nucleated cells discriminates CTCL from benign inflammatory skin diseases (Psoriasis, ED, Non malignant skin (nml)) (*p<0.05, ***p<0.001).

FIG. 9A shows patients diagnosed for CTCL using high throughput sequencing (HTS) in various stages, who were found negative for clonality by conventional TCRγ PCR. HTS and TCRγ PCR were carried out on skin biopsies from 39 CTCL patients; HTS identified clones in 39/39 CTCL patients compared to TCRγ PCR, which identified clonal populations in 29/39 samples. The stages of the nine of the ten CTCL patients with clones detected by HTS but negative for clonality by TCRγ PCR are shown.

FIG. 9B shows individual samples that were detected for CTCL using HTS, as compared to those detected by TCRγ PCR. Failure of TCRγ PCR to detect clonality was not related to the level of the malignant T cell clone within the skin sample. The top TCRβ clone, expressed as the fraction of nucleated cells, is shown. CTCL data are shown along with psoriasis samples, which are included for comparison.

FIG. 9C shows aggregate data for samples that were detected for CTCL using HTS, as compared to those detected by TCRγ PCR. Aggregate data are shown along with psoriasis samples, which are included for comparison.

FIG. 9D shows a clinical photo and HTS results for Patient 541, who had pathology proven stage IB CTCL, but in whom TCRγ PCR did not detect a clonal population.

FIG. 9E shows a clinical photo and HTS results for patient 347. TCRγ PCR was negative and pathology was equivocal, but HTS results of TCRB demonstrated a clear malignant clone.

FIG. 9F shows TCR γ PCR results for two biopsies from patient 551. In total, four skin biopsies were sent for HTS, and three biopsy samples were studied by TCRγ PCR. All samples were negative for clonality by TCRγ PCR, but 4/4 were positive for clonality by HTS.

FIG. 9G shows TCRγ PCR results for two biopsies of patient 551. The results for the third sample are shown in FIG. 7. Asterisks indicate peaks noted by the pathologist within the expected areas, but none were judged significant enough to designate as a clonal population.

FIG. 9H shows that HTS of TCRVβ demonstrated the presence of two distinct Vβ clonal sequences, denoting the presence of either a single malignant clone with two rearranged TCRβ alleles or two separate malignant T cell clones.

FIG. 9I shows results of HTS of TCRγ, which demonstrated the presence of four dominant gamma chain clones in all four biopsies (black circles), confirming that there were two clonal malignant T cell populations in this patient, each with one rearranged TCRβ allele and two rearranged TCRγ alleles. The five most frequent benign T cell TCRγ sequences are also shown for comparison (white circles). ***p<0.001.

FIG. 10A illustrates that HTS discriminates CTCL recurrences from benign inflammation, provides accurate assessment of responses to therapy and facilitates early diagnosis of disease recurrence in both the skin and blood of patients with CTCL. Patient 247 had a history of stage IB CTCL, subsequent developed leukemic involvement and was treated with low dose alemtuzumab. FIG. 10A shows that prior to therapy, TCRVβ HTS demonstrated a clear malignant clone in blood.

FIG. 10B shows a photograph of patient 247. The patient initially improved on alemtuzumab, and then developed the skin eruption as shown. Histopathology of the lesional skin was suggestive of a drug hypersensitivity reaction, but a T cell dyscrasia could not be ruled out.

FIG. 10C shows a graph of HTS results of blood and lesional skin, which showed clearance of the malignant T cell clone from blood and skin, confirming this was a benign inflammatory dermatitis. Bactrim was discontinued and the eruption completely resolved with topical steroids and narrow band UVB therapy.

FIG. 10D shows a 3D histogram of diverse populations of T cells remaining within the skin of alemtuzumab treated patients. TCRVβ HTS of the skin of patient 247 while on alemtuzumab is shown. This patient had no circulating T or B cells but a diverse population of T cells remained in skin.

FIG. 10E shows a photograph of skin of patient 247.

FIG. 10F shows a graph of HTS results of the malignant clone and benign T cells (as % T cells). HTS allows longitudinal observation of disease activity over time and assesses responses to therapy. Patient 409, who had Stage IIA CTCL, was studied by HTS in 2012, after treatment with electron beam and brachytherapy and again in 2014, after initiation of gemcitabine. HTS demonstrated an identical clonal T-cell population in the skin at both time points, reduced but still frequent after gemcitabine therapy. The malignant clone (black circle) and three most frequent benign T cell clones (white circles) are shown.

FIG. 10G shows a photograph of patient 425. Patient 425 had recalcitrant stage IIB CTCL with CD30⁺ large cell transformation. She underwent stem cell transplantation (SCT) and appeared well until she developed a new right chest lesion 10 months after SCT.

FIG. 10H a graph of HTS results of a malignant clone and benign T cells (as % T cells). HTS provides early diagnosis of disease recurrence. HTS prior to SCT demonstrated that presence of a malignant T cell clone. Biopsy of the lesion post SCT demonstrated recurrence of the same malignant T cell clone in the skin. The malignant T cell clone (black circles) and the three most frequent benign T cell clones (white circle) are shown. Subsequent withdrawal of systemic immunosuppression and narrowband UVB therapy induced a complete remission.

FIG. 10I shows the top T cell clones as a fraction of total nucleated cells for clonal populations in patients with MF without evidence of blood disease (MF) and in patients with leukemic disease (L-CTCL) with known blood involvement. HTS allows accurate assessment of peripheral blood disease.

FIG. 11A shows a skin lesion of patient 539. Patient 539 was stage IIIB CTCL and had no evidence of peripheral blood disease by clinical flow analysis but was experiencing both patchy (LCT on histopathology) and nodular skin (MF on histopathology) skin lesions. The patient improved following local radiation therapy but subsequently developed a new tumor at a previously uninvolved site five months later.

In FIG. 11B(i), FIG. 11B(ii) and FIG. 11B(iii), HTS studies of three biopsies are shown. In patients with skin limited disease and no clinical involvement of peripheral blood, HTS demonstrates hematogenous spread of small numbers of malignant T cells. Clinical flow was negative for blood involvement on both dates (Jan. 16, 2013 and Jun. 19, 2013). HTS demonstrated the same clonal T cell population in all three skin biopsies and analysis of the blood demonstrated the presence of small numbers of malignant T cells in the peripheral blood at the time new skin lesions were developing.

FIG. 11C shows the T cell clone in the three skin biopsies as % T cells in skin and as a fraction of total T cells in blood. The numbers of T cells carrying these sequences are indicated above the bars.

FIG. 11D shows a photograph of skin of Patient 418, who had longstanding stage IIB folliculotropic CTCL.

FIG. 11E shows a photograph of skin of Patient 418 with recent thickening of existing skin lesions and new areas of disease.

FIG. 11F shows the percent of total T cells of a malignant clone (a total of 25,205 cells of the malignant clone). Clinical flow analyses were negative for peripheral blood involvement. HTS studies of the skin and blood during the development of the skin lesions demonstrated a clear malignant T cell clone in lesional skin that was also found in low numbers in peripheral blood.

FIG. 11G shows a photograph of skin of a patient. Patient 317 had a longstanding history of large plaque parapsoriasis since childhood for over 40 years. The diagnosis of MF was finally made in 2007. In 2014, the presented with a 1.5 year history of worsening disease with new areas of involvement.

FIG. 11H shows results of HTS analysis in skin and blood samples of patient 317. HTS demonstrated a malignant T cell clone in the skin (702 cells of the malignant T cell clone; greater than 2% of T cells in skin sample), and small numbers were also demonstrated in peripheral blood. Clinical flow analyses showed no evidence of peripheral blood disease.

FIG. 12A shows counts of a malignant clone and benign infiltrating T cells for a patient. The TCR Vβ HTS profile is shown for patient 541; both the malignant clone and benign infiltrating T cells in the skin lesions are evaluated by this technique.

FIG. 12B shows CDR3 length analysis, which provides a rapid assay of T cell diversity via HTS. Shown are the results of TCRγ HTS of lesional skin from a patient with stage III CTCL. The CDR3 lengths of all T cells (left panel, including the malignant clone) and benign infiltrating T cells only (right panel) are shown. The two rearranged TCRγ allele sequences of the malignant clone are indicated by asterisks. A healthy diverse population of benign infiltrating T cells was present.

FIG. 12C shows the top two most frequent Vγ sequences in each patient sample. Malignant αβ T cell clones of 33 CTCL patients were analyzed by TCRγ HTS. 27 patients had two rearranged TCRγ alleles, as evidenced by the presence of two similarly frequent TCRγ sequences.

FIG. 12D shows results of TCRγ HTS for six patients, who had a single rearranged TCRγ allele, as evidenced by only a single high-frequency TCRγ sequence.

FIG. 12E shows the number of patients with bi-allelic or mono-allelic TCRγ. HTS T cells therefore had on average 1.8 rearranged TCRγ alleles, a proportion characteristic of mature T cells. Prior studies demonstrated that mature αβ T cells have on average 1.8 rearranged TCRγ alleles (9).

FIG. 12F shows a diagram for identification of the TCR Vβ chain by HTS and subsequent immunostaining using commercially available TCR Vβ antibodies, which allows study of clonal malignant T cells.

FIG. 12G and FIG. 12H show immunostaining of malignant T cells with TCR Vβ-specific antibodies in two patients with stage IIB MF. The patient shown in FIG. 12H had a high abundant clone and larger cells as a result of large cell transformation.

FIG. 13 shows TCR γ chain PCR results for a third biopsy from patient 551. Asterisks indicate peaks noted by the pathologist within the expected areas, but none were judged significant enough to designate as a clonal population. Clinical photos and PCR results from other biopsies are shown in FIGS. 9F-9I.

### DETAILED DESCRIPTION OF THE INVENTION

Definitions

Terms used in the claims and specification are defined as set forth below unless otherwise specified.

As used herein, adaptive immune receptor (AIR) refers to an immune cell receptor, e.g., a T cell receptor (TCR) or a B cell receptor (BCR) found in mammalian cells. In certain embodiments, the adaptive immune receptor is encoded by a TCRB, TCRG, TCRA, TCRD, IGH, IGK, and IGL gene or gene segment.

The term “primer,” as used herein, refers to an oligonucleotide sequence capable of acting as a point of initiation of DNA synthesis under suitable conditions. Such conditions include those in which synthesis of a primer extension product complementary to a nucleic acid strand is induced in the presence of four different nucleoside triphosphates and an agent for extension (e.g., a DNA polymerase or reverse transcriptase) in an appropriate buffer and at a suitable temperature.

In some embodiments, as used herein, the term “gene” refers to the segment of DNA involved in producing a polypeptide chain, such as all or a portion of a TCR or Ig polypeptide (e.g., a CDR3-containing polypeptide); it includes regions preceding and following the coding region “leader and trailer” as well as intervening sequences (introns) between individual coding segments (exons), regulatory elements (e.g., promoters, enhancers, repressor binding sites and the like), or recombination signal sequences (RSSs), as described herein.

The nucleic acids of the present embodiments also referred to herein as polynucleotides, and including oligonucleotides, can be in the form of RNA or in the form of DNA, including cDNA, genomic DNA, and synthetic DNA. The DNA can be double-stranded or single-stranded, and if single stranded can be the coding strand or non-coding (anti-sense) strand. A coding sequence which encodes a TCR or an IG or a region thereof (e.g., a V region, a D segment, a J region, a C region, etc.) for use according to the present embodiments can be identical to the coding sequence known in the art for any given TCR or immunoglobulin gene regions or polypeptide domains (e.g., V-region domains, CDR3 domains, etc.), or can be a different coding sequence, which as a result of the redundancy or degeneracy of the genetic code, encodes the same TCR or immunoglobulin region or polypeptide.

As used herein, “lymphoid malignancy” or “lymphoid neoplasm” refer to any malignant or cancerous condition affecting a lymphoid cell. All known types of leukemias and lymphomas belong to the classification of “lymphoid malignancies/lymphoid neoplasm.”

Exemplary mature B-cell neoplasms include, but are not limited to, chronic lymphocytic leukemia/small lymphocytic lymphoma, B-cell prolymphocytic leukemia, lymphoplasmacytic lymphoma, cplenic marginal zone lymphoma, hairy cell leukemia, splenic B-cell lymphoma, plasma cell neoplasms, extranodal marginal zone lymphoma, nodal marginal zone lymphoma, follicular lymphoma, primary cutaneous follicle center lymphoma, mantle cell lymphoma, diffuse large B-cell lymphoma (DLBCL), DLBCL associated with chronic inflammation, lymphomatoid granulomatosis, primary mediastinal large B-cell lymphoma, intravascular large B-cell lymphoma, ALK-positive large B-cell lymphoma, plasmablastic lymphoma, large B-cell lymphoma arising in HHV-8-associated multicentric Castleman's disease, Burkitt's lymphoma, B-cell lymphoma, unclassifiable.

Examples of mature T-cell and natural killer (NK) cell neoplasms include, but are not limited to, T-cell prolymphocytic leukemia, T-cell large granular lymphocytic leukemia, chronic lymphoproliferative disorder of NK cells, aggressive NK cell leukemia, EBV-positive T-cell lymphophoproliferative diseases of childhood, adult T-cell leukemia/lymphoma, extranodal NK/T-cell lymphoma (nasal type), enteropathy-type T-cell lymphoma, hepatosplenic T-cell lymphoma, subcutaneous panniculitis-like T-cell lymphoma, mycosis fungoides, Sezary's syndrome, primary cutaneous CD30+ T-cell lymphoproliferative disorders, primary cutaneous peripheral T-cell lymphomas, rare subtypes, peripheral T-cell lymphoma, angioimmunoblastic T-cell lymphoma (ALK+), and anaplastic large cell lymphoma (ALK-).

Other types of lymphoid neoplasms include precursor B- and T-cell neoplasms and immunodeficiency-associated lymphoproliferative disorders (lymphoproliferative diseases associated with primary immune disorders, lymphomas associated with HIV infection, posttransplant lymphoproliferative disorders (PTLD), and other iatrogenic immunodeficiency-associated lymphoproliferative disorders).

The National Comprehensive Cancer Network (NCCN) has also categorized the following as Non-Hodgkin's lymphomas: Chronic Lymphocytic leukemia/small lymphocytic lymphoma, follicular lymphoma, marginal zone lymphomas, (gastric MALT lymphoma, non-gastric MALT lymphoma, nodal marginal zone lymphoma, splenic marginal zone lymphoma), mantle cell lymphoma, diffuse large B-cell lymphoma, Burkitt lymphoma, lymphoblastic lymphoma, AIDS related B-cell lymphomas, primary cutaneous B-cell lymphomas, peripheral T-cell lymphoma, non-cutaneous, mycosis fungoides/Sezary Syndrome, adult T-cell leukemia/lymphoma, extranodal NK/T-cell lymphoma, post-transplant lymphoproliferative disorders, T-cell prolymphocytic leukemia, and hairy cell leukemia.

As used herein CTCL refers to a heterogeneous collection of non-Hodgkin's lymphomas derived from skin tropic T cells. CTCL encompasses skin limited variants, such as mycosis fungoides (MF), and leukemic forms of the disease, including Sézary syndrome. CTCL is divided into the following types: Mycosis fungoides (MF), Pagetoid reticulosis, Sézary syndrome, Granulomatous slack skin, Lymphomatoid papulosis, Pityriasis lichenoides chronica, Pityriasis lichenoides et varioliformis acuta, CD30+ cutaneous T-cell lymphoma, Secondary cutaneous CD30+ large cell lymphoma, Non-mycosis fungoides CD30− cutaneous large T-cell lymphoma, Pleomorphic T-cell lymphoma, Lennert lymphoma, Subcutaneous T-cell lymphoma, Angiocentric lymphoma, and Blastic NK-cell lymphoma.

The term “ameliorating” refers to any therapeutically beneficial result in the treatment of a disease state, e.g., a disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques can be used for recombinant technology, molecular biological, microbiological, chemical syntheses, chemical analyses, pharmaceutical preparation, formulation, and delivery, and treatment of patients.

Unless the context requires otherwise, throughout the present specification and claims, the word “comprise” and variations thereof, such as, “comprises” and “comprising” are to be construed in an open, inclusive sense, that is, as “including, but not limited to.” By “consisting of” is meant including, and typically limited to, whatever follows the phrase “consisting of.” By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are required and can or cannot be present depending upon whether or not they affect the activity or action of the listed elements.

It must be noted that, as used in the specification and the appended claims, the singular forms “a,” “an” and “the” include plural referents unless the context clearly dictates otherwise.

As used herein, in particular embodiments, the terms “about” or “approximately” when preceding a numerical value indicates the value plus or minus a range of 5%, 6%, 7%, 8% or 9%, or greater, etc. In other embodiments, the terms “about” or “approximately” when preceding a numerical value indicates the value plus or minus a range of 10%, 11%, 12%, 13% or 14%, or greater, etc. In yet other embodiments, the terms “about” or “approximately” when preceding a numerical value indicates the value plus or minus a range of 15%, 16%, 17%, 18%, 19% or 20%, or greater, etc.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

Methods of the Invention

The invention includes using compositions and methods for quantitative detection of sequences of substantially all possible TCR and IG gene rearrangements that can be present in a sample containing lymphoid cell DNA. Methods for determining TCR and/or IG repertoire diversity are described further in U.S. Ser. No. 12/794,507, filed on Jun. 4, 2010, International App. No. PCT/US2013/062925, filed on Oct. 1, 2013, U.S. Ser. No. 13/217,126, filed on Aug. 24, 2011, International App. No. PCT/US2011/049012, filed on Aug. 24, 2011, U.S. Ser. No. 14/381,967, filed Aug. 8, 2014, and U.S. Ser. No. 61/949,069 filed Mar. 6, 2014 which are both incorporated by reference in their entireties.

The methods of the invention include 1) use of primers and methods for controlled and unbiased multiplex polymerase chain reaction (PCR) amplification of all possible CDR3 regions that might be present in genomic DNA (or cDNA) derived from a given immune receptor (IG or TCR) locus within each lymphocyte in a blood, bone marrow, or tissue sample, 2) high throughput sequencing of the amplified products, 3) refined and formidable computational analysis of the raw sequence data output to eliminate “noise”, extract signal, trouble shoot technological artifacts, and validate process control from sample receipt through sequence delivery, and 4) highly accurate and sensitive detection of CTCL in a sample based on high throughput sequencing of TCR repertoire of the sample.

Samples

Samples used in the methods of the invention can include, any tissue from a subject where there is a lymphoid infiltrate in the tissue, and the lymphoid infiltrate can be malignant or benign.

The subject is a mammalian subject, for example, a human subject.

Samples can include a bodily fluid from a subject, such as a peripheral blood sample. The blood sample can be about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 mL or greater. Other examples of samples include, but not limited to, urine, blood and blood plasma, saliva, internal body fluids, etc.

The sample can be obtained by a health care provider, for example, a physician, physician assistant, nurse, dermatologist, rheumatologist, dentist, paramedic, surgeon, or a research technician. Multiple samples from a subject can be obtained (e.g., pre and post treatment).

The sample can be a biopsy. In other embodiments, the sample is a skin biopsy of a subject with a skin condition, such as CTCL, psoriasis, eczematous dermatitis (ED), or from a healthy subject. In some embodiments, the biopsy can be from another tissue or organ, for example, ovary, breast, brain, liver, lung, heart, colon, kidney, or bone marrow. Any biopsy technique used by those skilled in the art can be used for isolating a sample from a subject. For example, a biopsy can be an open biopsy, in which general anesthesia is used. The biopsy can be a closed biopsy, in which a smaller cut is made than in an open biopsy. The biopsy can be a core or incisional biopsy, in which part of the tissue is removed. The biopsy can be an excisional biopsy, in which attempts to remove an entire lesion are made. The biopsy can be a fine needle aspiration biopsy, in which a sample of tissue or fluid is removed with a needle.

The sample includes T-cells and/or B-cells. T-cells (T lymphocytes) include, for example, cells that express T cell receptors. T-cells include Helper T cells (effector T cells or Th cells), cytotoxic T cells (CTLs), memory T cells, and regulatory T cells. The sample can include one or more expanded clones, including a dominant clone (e.g., a top T cell clone), among a number of benign T cells or a total number of nucleated cells. The sample can include at least 1,000, at least 10,000, at least 100,000, at least 250,000, at least 500,000, at least 750,000, or at least 1,000,000 T-cells.

B-cells include, for example, plasma B cells, memory B cells, B1 cells, B2 cells, marginal-zone B cells, and follicular B cells. B-cells can express immunoglobulins (Igs, antibodies, B cell receptor). The sample can include one or more expanded clones, including a dominant clone (e.g., a top B cell clone), among a number of benign B cells or a total number of nucleated cells. The sample can include a single B cell in some applications or more generally at least 1,000, at least 10,000, at least 100,000, at least 250,000, at least 500,000, at least 750,000, or at least 1,000,000 B-cells.

The sample can include nucleic acid molecules extracted from a cell, for example, DNA (e.g., genomic DNA or mitochondrial DNA) or RNA (e.g., messenger RNA or microRNA). The nucleic acid can be cell-free DNA or RNA. In other embodiments, the sample comprises complementary DNA (cDNA) that has been reverse transcribed from mRNA. In the methods of the provided invention, the amount of RNA or DNA from a subject that can be analyzed includes, for example, as low as a single cell in some applications and as many as 10 millions of cells or more, translating to a range of DNA of 6 pg-60 μg, and RNA of approximately 1 pg-10 μg.

In some embodiments, skin samples can be obtained from patients undergoing cosmetic surgery procedures. In other embodiments, both blood and lesional skin samples can be obtained from patients with CTCL and from patients with non-malignant skin conditions, or healthy, normal skin.

Cells

B cells and T cells can be obtained from a biological sample, such as from a variety of tissues, solid tumor samples, and biological fluid samples, including skin tissue, bone marrow, thymus, lymph glands, lymph nodes, peripheral tissues and peripheral blood.

Any peripheral tissue can be sampled for the presence of B and T cells and is therefore contemplated for use in the methods described herein. Tissues and biological fluids from which adaptive immune cells may be obtained include, but are not limited to skin, epithelial tissues, colon, spleen, a mucosal secretion, oral mucosa, intestinal mucosa, vaginal mucosa or a vaginal secretion, cervical tissue, ganglia, saliva, cerebrospinal fluid (CSF), bone marrow, cord blood, serum, serosal fluid, plasma, lymph, urine, ascites fluid, pleural fluid, pericardial fluid, peritoneal fluid, abdominal fluid, culture medium, conditioned culture medium or lavage fluid. In certain embodiments, adaptive immune cells may be isolated from an apheresis sample. Peripheral blood samples may be obtained by phlebotomy from subjects. Peripheral blood mononuclear cells (PBMC) are isolated by techniques known to those of skill in the art, e.g., by Ficoll-Hypaque® density gradient separation. In certain embodiments, whole PBMCs are used for analysis.

In other embodiments, the sample comprises solid tumor tissue, a circulating blood mononuclear cell fraction, or cells collected from urinary sediment.

In certain related embodiments, preparations that comprise predominantly lymphocytes (e.g., T and B cells) or that comprise predominantly T cells or predominantly B cells, may be prepared. In other related embodiments, specific subpopulations of T or B cells may be isolated prior to analysis using the methods described herein. Various methods and commercially available kits for isolating different subpopulations of T and B cells are known in the art and include, but are not limited to, subset selection immunomagnetic bead separation or flow immunocytometric cell sorting using antibodies specific for one or more of any of a variety of known T and B cell surface markers. Illustrative markers include, but are not limited to, one or a combination of CD2, CD3, CD4, CD8, CD14, CD19, CD20, CD25, CD28, CD45RO, CD45RA, CD54, CD62, CD62L, CDw137 (41BB), CD154, GITR, FoxP3, CD54, and CD28. For example, and as is known to the skilled person, cell surface markers, such as CD2, CD3, CD4, CD8, CD14, CD19, CD20, CD45RA, and CD45RO may be used to determine T, B, and monocyte lineages and subpopulations in flow cytometry. Similarly, forward light-scatter, side-scatter, and/or cell surface markers such as CD25, CD62L, CD54, CD137, and CD154 may be used to determine activation state and functional properties of cells.

Illustrative combinations useful in certain of the methods described herein may include CD8⁺CD45RO⁺ (memory cytotoxic T cells), CD4⁺CD45RO⁺ (memory T helper), CD8⁺CD45RO⁻ (CD8⁺CD62L⁻CD45RA⁺ (naïve-like cytotoxic T cells); CD4⁺CD25⁻CD62L^{hi}GITR⁺FoxP3⁺ (regulatory T cells). Illustrative antibodies for use in immunomagnetic cell separations or flow immunocytometric cell sorting include fluorescently labeled anti-human antibodies, e.g., CD4 FITC (clone M-T466, Miltenyi Biotec), CD8 PE (clone RPA-T8, BD Biosciences), CD45RO ECD (clone UCHL-1, Beckman Coulter), and CD45RO APC (clone UCHL-1, BD Biosciences). Staining of total PBMCs may be done with the appropriate combination of antibodies, followed by washing cells before analysis. Lymphocyte subsets can be isolated by fluorescence activated cell sorting (FACS), e.g., by a BD FACSAria™ cell-sorting system (BD Biosciences) and by analyzing results with FlowJo™ software (Treestar Inc.), and also by conceptually similar methods involving specific antibodies immobilized to surfaces or beads.

### Nucleic Acid Extraction

In some embodiments, total genomic DNA can be extracted from cells by methods known to those of skill in the art. Examples include using the QIAamp® DNA blood Mini Kit (QIAGEN®). The approximate mass of a single haploid genome is 3 pg. Preferably, at least 100,000 to 200,000 cells are used for analysis of diversity, i.e., about 0.6 to 1.2 μg DNA from diploid T cells. Using PBMCs as a source, the number of T cells can be estimated to be about 30% of total cells.

In some embodiments, RNA can be extracted from cells in a sample, such as a sample of blood, lymph, tissue, or other sample from a subject known to contain lymphoid cells, using standard methods or commercially available kits known in the art. In other embodiments, cDNA can be transcribed from mRNA obtained from the cells and then used as templates in a multiplex PCR.

Alternatively, total nucleic acid can be isolated from cells, including both genomic DNA and mRNA. If diversity is to be measured from mRNA in the nucleic acid extract, the mRNA can be converted to cDNA prior to measurement. This can readily be done by methods of one of ordinary skill, for example, using reverse transcriptase according to known procedures.

In certain embodiments, DNA can be isolated from frozen, OCT embedded or formalin fixed paraffin embedded (FFPE) skin samples. For OCT embedded tissue samples, cryosections can be cut and DNA extraction can be carried extracted using known techniques. For FFPE samples, paraffin is first removed from the tissue scrolls and DNA can then be extracted by known techniques.

### Multiplex Quantitative PCR

Multiplex quantitative PCR is described herein and in Robins et al., 2009 Blood 114, 4099; Robins et al., 2010 Sci. Translat. Med. 2:47ra64; Robins et al., 2011 J. Immunol. Meth. doi:10.1016/j.jim.2011.09. 001; Sherwood et al. 2011 Sci. Translat. Med. 3:90ra61; U.S. Ser. No. 13/217,126, U.S. Ser. No. 12/794,507, WO/2010/151416, WO/2011/106738 (PCT/US2011/026373), WO2012/027503 (PCT/US2011/049012), U.S. Ser. No. 61/550,311, and U.S. Ser. No. 61/569,118, which are each incorporated by reference in its entirety. The present methods involve a single multiplex PCR method using a set of forward primers that specifically hybridize to V segments and a set of reverse primers that specifically hybridize to the J segments of a TCR or IG locus, where a single multiplex PCR reaction using the primers allows amplification of all the possible VJ (and VDJ) combinations within a given population of T or B cells.

A single multiplex PCR system can be used to amplify rearranged adaptive immune cell receptor loci from genomic DNA, preferably from a CDR3 region. In certain embodiments, the CDR3 region is amplified from a TCRA, TCRG, TCRG or TCRD CDR3 region or similarly from an IGH or IGL (lambda or kappa) locus. Compositions are provided that comprise a plurality of V-segment and J-segment primers that are capable of promoting amplification in a multiplex polymerase chain reaction (PCR) of substantially all productively rearranged adaptive immune receptor CDR3-encoding regions in the sample for a given class of such receptors to produce a multiplicity of amplified rearranged DNA molecules from a population of T cells (for TCR) or B cells (for IG) in the sample. In certain embodiments, primers are designed so that each amplified rearranged DNA molecule is less than 600 nucleotides in length, thereby excluding amplification products from non-rearranged adaptive immune receptor loci.

In some embodiments, the method uses two pools of primers to provide for a highly multiplexed, single tube PCR reaction. A “forward” pool of primers can include a plurality of V-segment oligonucleotide primers used as “forward” primers and a plurality of J-segment oligonucleotide primers used as “reverse” primers. In other embodiments, J-segment primers can be used as “forward” primers, and V-segment can be used “reverse” primers. In some embodiments, an oligonucleotide primer that is specific to (e.g., having a nucleotide sequence complementary to a unique sequence region of) each V-region encoding segment (“V segment) in the respective TCR or IG gene locus can be used. In other embodiments, primers targeting a highly conserved region are used to simultaneously amplify multiple V segments or multiple J segments, thereby reducing the number of primers required in the multiplex PCR. In certain embodiments, the J-segment primers anneal to a conserved sequence in the joining (“J”) segment.

Each primer can be designed such that a respective amplified DNA segment is obtained that includes a sequence portion of sufficient length to identify each J-segment unambiguously based on sequence differences amongst known J-region encoding gene segments in the human genome database, and also to include a sequence portion to which a J-segment specific primer can anneal for resequencing. This design of V- and J-segment specific primers enables direct observation of a large fraction of the somatic rearrangements present in the adaptive immune receptor gene repertoire within an individual. This feature in turn enables rapid comparison of the TCR and/or IG repertoires in individuals pre-transplant and post-transplant, for example.

In one embodiment, the present disclosure provides a plurality of V-segment primers and a plurality of J-segment primers, wherein the plurality of V-segment primers and the plurality of J-segment primers amplify all or substantially all combinations of the V- and J-segments of a rearranged immune receptor locus. In some embodiments, the method provides amplification of substantially all of the rearranged adaptive immune receptor (AIR) sequences in a lymphoid cell and is capable of quantifying the diversity of the TCR or IG repertoire of at least 10⁶, 10⁵, 10⁴, or 10³ unique rearranged AIR sequences in a sample. “Substantially all combinations” can refer to at least 80%, 85%, 90%, 91%, 92%, 93%, 94%, 95%, 96%, 97%, 98%, 99% or more of all the combinations of the V- and J-segments of a rearranged immune receptor locus. In certain embodiments, the plurality of V-segment primers and the plurality of J-segment primers amplify all of the combinations of the V- and J-segments of a rearranged adaptive immune receptor locus.

In general, a multiplex PCR system can use 2, 3, 4, 5, 6, 7, 8, 9, 10, 11, 12, 13, 14, 15, 16, 17, 18, 19, 20, 21, 22, 23, 24, or 25, and in certain embodiments, at least 26, 27, 28, 29, 30, 31, 32, 33, 34, 35, 36, 37, 38, or 39, and in other embodiments 40, 41, 42, 43, 44, 45, 46, 47, 48, 49, 50, 51, 52, 53, 54, 55, 56, 57, 58, 59, 60, 65, 70, 75, 80, 85, or more forward primers, in which each forward primer specifically hybridizes to or is complementary to a sequence corresponding to one or more V region segments. The multiplex PCR system also uses at least 2, 3, 4, 5, 6, or 7, and in certain embodiments, 8, 9, 10, 11, 12 or 13 reverse primers, or 14, 15, 16, 17, 18, 19, 20, 21, 22, 23, 24, or 25 or more primers, in which each reverse primer specifically hybridizes to or is complementary to a sequence corresponding to one or more J region segments. In some embodiments, each reverse J primer is specific to a different J gene segment. In other embodiments, there is no common J primer that binds to all J gene segments.

The V segment and J segment primers have certain characteristics to amplify the total diversity of TCR or IG repertoires. In certain embodiments, the V segment primers have similar melting temperatures within a range of 0.1° C., 0.2° C., 0.3° C., 0.4° C., 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.9° C., 2.0° C., 2.1° C., 2.2° C., 2.3° C., 2.4° C., 2.5° C., 2.6° C., 2.7° C., 2.8° C., 2.9° C., 3.0° C., 3.1° C., 3.2° C., 3.3° C., 3.4° C., 3.5° C., 3.6° C., 3.7° C., 3.8° C., 3.9° C., 4.0° C., 4.5° C., 5.0° C. In some embodiments, the J segment primers have similar melting temperatures within a range of 0.1° C., 0.2° C., 0.3° C., 0.4° C., 0.5° C., 0.6° C., 0.7° C., 0.8° C., 0.9° C., 1.0° C., 1.1° C., 1.2° C., 1.3° C., 1.4° C., 1.5° C., 1.6° C., 1.7° C., 1.8° C., 1.9° C., 2.0° C., 2.1° C., 2.2° C., 2.3° C., 2.4° C., 2.5° C., 2.6° C., 2.7° C., 2.8° C., 2.9° C., 3.0° C., 3.1° C., 3.2° C., 3.3° C., 3.4° C., 3.5° C., 3.6° C., 3.7° C., 3.8° C., 3.9° C., 4.0° C., 4.5° C., 5.0° C.

In certain embodiments, the plurality of V segment and J segment primers are not consensus primers. The V segment and J segment primers are not universal, degenerate primers. In some embodiments, each V segment primer is complementary to a single V segment or a family of V segments. In some embodiments, each J segment primer is complementary to a single J segment or a family of J segments. In other embodiments, each J segment primer is complementary and specific to a single J segment gene.

In other embodiments, the plurality of V segment and J segment primers sit outside a region of untemplated deletions in the TCR or IG locus. In some embodiments, the 3′ end of the V segment primers are complementary to a target region that is at least 10, 11, 12, 13, 14, 15, 16, 17, 18, 19, 20, 21, 22, 23, 24, 25, 26, 27, 28, 29, 30 or more nucleotides upstream from the V-RSS. In some embodiments, the 3′ end of the J segment primers are complementary to a target region that is at least 10, 11, 12, 13, 14, 15, 16, 17, 18, 19, 20, 21, 22, 23, 24, 25, 26, 27, 28, 29, 30 or more nucleotides downstream from the J-RSS.

Various combinations of V and J segment primers can be used to amplify the full diversity of TCR and IG sequences in a repertoire. For details on the multiplex PCR system, including exemplary primer oligonucleotide sequences for amplifying substantially all TCR and/or IG sequences, see, e.g., Robins et al., 2009 Blood 114, 4099; Robins et al., 2010 Sci. Translat. Med. 2:47ra64; Robins et al., 2011 J. Immunol. Meth. doi:10.1016/j.jim.2011.09. 001; Sherwood et al. 2011 Sci. Translat. Med. 3:90ra61; U.S. Ser. No. 13/217,126, U.S. Ser. No. 12/794,507, WO/2010/151416, WO/2011/106738 (PCT/US2011/026373), WO2012/027503 (PCT/US2011/049012), U.S. Ser. No. 61/550,311, U.S. Ser. No. 61/569,118, WO/2013/188831 (PCT/US2013/045994), which is each incorporated by reference in its entirety.

Oligonucleotides or polynucleotides that are capable of specifically hybridizing or annealing to a target nucleic acid sequence by nucleotide base complementarity can do so under moderate to high stringency conditions. For purposes of illustration, suitable moderate to high stringency conditions for specific PCR amplification of a target nucleic acid sequence would be between 25 and 80 PCR cycles, with each cycle consisting of a denaturation step (e.g., about 10-30 seconds (s) at greater than about 95° C.), an annealing step (e.g., about 10-30s at about 60-68° C.), and an extension step (e.g., about 10-60s at about 60-72° C.), optionally according to certain embodiments with the annealing and extension steps being combined to provide a two-step PCR. As would be recognized by the skilled person, other PCR reagents can be added or changed in the PCR reaction to increase specificity of primer annealing and amplification, such as altering the magnesium concentration, optionally adding DMSO, and/or the use of blocked primers, modified nucleotides, peptide-nucleic acids, and the like.

In certain embodiments, nucleic acid hybridization techniques can be used to assess hybridization specificity of the primers described herein. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5×and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C.-70° C.

In certain embodiments, the primers are designed not to cross an intron/exon boundary. The forward primers in certain embodiments anneal to the V segments in a region of relatively strong sequence conservation between V segments so as to maximize the conservation of sequence among these primers. Accordingly, this minimizes the potential for differential annealing properties of each primer, and so that the amplified region between V and J primers contains sufficient TCR or Ig V sequence information to identify the specific V gene segment used. In one embodiment, the J segment primers hybridize with a conserved element of the J segment and have similar annealing strength. In one particular embodiment, the J segment primers anneal to the same conserved framework region motif. In certain embodiments, the J segment primers have a melting temperature range within 10° C., 7.5° C., 5° C., or 2.5° C. or less.

Oligonucleotides (e.g., primers) can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

A primer is preferably a single-stranded oligonucleotide. The appropriate length of a primer depends on the intended use of the primer but typically ranges from 6 to 50 nucleotides, 15-50 nucleotides, or in certain embodiments, from 15-35 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

As described herein, primers can incorporate additional features which allow for the detection or immobilization of the primer, but do not alter the basic property of the primer, that of acting as a point of initiation of DNA synthesis. For example, primers can contain an additional nucleic acid sequence at the 5′ end, which does not hybridize to the target nucleic acid, but which facilitates cloning, detection, or sequencing of the amplified product. The region of the primer which is sufficiently complementary to the template to hybridize is referred to herein as the hybridizing region.

As used herein, a primer is “specific” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences which contain the target primer binding sites. In other terms, the primers of the invention are each complementary to a target sequence and can include 1, 2, or more mismatches without reducing complementarity or hybridization of the primer to the target sequence.

In particular embodiments, primers for use in the methods described herein comprise or consist of a nucleic acid of at least about 15 nucleotides long that has the same sequence as, or is substantially complementary to, a contiguous nucleic acid sequence of the target V or J segment. Longer primers, e.g., those of about 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80 or more nucleotides long that have the same sequence as, or sequence complementary to, a contiguous sequence of the target V or J segment, will also be of use in certain embodiments. Various mismatches (1, 2, 3, or more) to the target sequence can be contemplated in the primers, while preserving complementarity to the target V or J segment. All intermediate lengths of the aforementioned primers are contemplated for use herein. As would be recognized by the skilled person, the primers can have additional sequence added (e.g., nucleotides that cannot be the same as or complementary to the target V or J segment), such as restriction enzyme recognition sites, adaptor sequences for sequencing, bar code sequences, and the like (see e.g., primer sequences provided herein and in the sequence listing). Therefore, the length of the primers can be longer, such as 55, 56, 57, 58, 59, 60, 65, 70, 75, or 80 or more nucleotides in length or more, depending on the specific use or need.

For example, in one embodiment, the forward and reverse primers are both modified at the 5′ end with the universal forward primer sequence compatible with a DNA sequencing nucleic acid sequence. Such universal primers sequences can be adapted to those used in the Illumina GAII single-end read sequencing system. Exemplary universal primer sequences and sequencing oligonucleotides are provided in U.S. Ser. No. 13/217,126, U.S. Ser. No. 12/794,507, PCT/US2011/049012, PCT/US2013/045994, which are incorporated by reference in their entireties.

In some embodiments, the forward and reverse primers are both modified at the 5′ end with an adaptor sequence that is not complementary to the V-segment, J-segment, or C-segment (target sequence) and can be used as a region complementary to a second set of primers or a sequencing oligonucleotide. The adaptor sequence may include one or more other sequences (barcode sequence, random sequences, or other sequencing oligonucleotide sequences) for additional amplification reactions, quantification purposes or sequencing purposes.

As would be recognized by the skilled person, in certain embodiments, other modifications may be made to the primers, such as the addition of restriction enzyme sites, fluorescent tags, and the like, depending on the specific application.

Also contemplated are adaptive immune receptor V-segment or J-segment oligonucleotide primer variants that can share a high degree of sequence identity to the oligonucleotide primers. Thus, in these and related embodiments, adaptive immune receptor V-segment or J-segment oligonucleotide primer variants can have substantial identity to the adaptive immune receptor V-segment or J-segment oligonucleotide primer sequences disclosed herein. For example, such oligonucleotide primer variants can comprise at least 70% sequence identity, preferably at least 75%, 80%, 85%, 90%, 91%, 92%, 93%, 94%, 95%, 96%, 97%, 98%, or 99% or higher sequence identity compared to a reference polynucleotide sequence such as the oligonucleotide primer sequences disclosed herein, using the methods described herein (e.g., BLAST analysis using standard parameters). One skilled in this art will recognize that these values can be appropriately adjusted to determine corresponding ability of an oligonucleotide primer variant to anneal to an adaptive immune receptor segment-encoding polynucleotide by taking into account codon degeneracy, reading frame positioning and the like. Typically, oligonucleotide primer variants will contain one or more substitutions, additions, deletions and/or insertions, preferably such that the annealing ability of the variant oligonucleotide is not substantially diminished relative to that of an adaptive immune receptor V-segment or J-segment oligonucleotide primer sequence that is specifically set forth herein. As also noted elsewhere herein, in preferred embodiments adaptive immune receptor V-segment and J-segment oligonucleotide primers are designed to be capable of amplifying a rearranged TCR or IGH sequence that includes the coding region for CDR3.

According to certain embodiments, the primers for use in the multiplex PCR methods of the present disclosure can be functionally blocked to prevent non-specific priming of non-T or B cell sequences. For example, the primers can be blocked with chemical modifications as described in U.S. Publication No. 2010/0167353.

In some embodiments, the V- and J-segment primers are used to produce a plurality of amplicons from the multiplex PCR reaction. In certain embodiments, the V-segment primer sand J-segment primers can produce at least 10⁶ amplicons representing the diversity of TCR or IG rearranged CDR3 molecules in the sample. In some embodiments, the amplicons range in size from 10, 20, 30, 40, 50, 75, 100, 200, 300, 400, 500, 600, 700, 800, 900, 1000, 1100, 1200, 1300, 1400, 1500 to 1600 nucleotides in length. In preferred embodiments, the amplicons have a size between 50-600 nucleotides in length.

According to non-limiting theory, these embodiments exploit current understanding in the art that once an adaptive immune cell (e.g., a T or B lymphocyte) has rearranged its adaptive immune receptor-encoding (e.g., TCR or Ig) genes, its progeny cells possess the same adaptive immune receptor-encoding gene rearrangement, thus giving rise to a clonal population that can be uniquely identified by the presence therein of rearranged (e.g., CDR3-encoding) V- and J-gene segments that can be amplified by a specific pairwise combination of V- and J-specific oligonucleotide primers as herein disclosed.

### Amplification Bias Control

Multiplex PCR assays can result in a bias in the total numbers of amplicons produced from a sample, given that certain primer sets are more efficient in amplification than others. To overcome the problem of such biased utilization of subpopulations of amplification primers, methods can be used that provide a template composition for standardizing the amplification efficiencies of the members of an oligonucleotide primer set, where the primer set is capable of amplifying rearranged DNA encoding a plurality of adaptive immune receptors (TCR or Ig) in a biological sample that comprises DNA from lymphoid cells.

Since accurate quantification of clones for CTCL detection is critical, an approach can be used to ensure minimal bias in multiplex PCR. See Carlson C S, Emerson R O, Sherwood A M, Desmarais C, Chung M-W, Parsons J M, et al. Using synthetic templates to design an unbiased multiplex PCR assay. Nature Communications. 2013; 4:2680, which is incorporated by reference. For example, each potential VDJ rearrangement of the TCRB locus contains one of thirteen J segments, one of 2 D segments and one of 52 V segments, many of which have disparate nucleotide sequences. In order to amplify all possible VDJ combinations, a single tube, multiplex PCR assay with 45 V forward and 13 J reverse primers was used. To remove potential PCR bias, every possible V-J pair was chemically synthesized as a template with specific barcodes. Id. These templates were engineered so as to be recognizable as non-biologic and have universal 3′ and 5′ ends to permit amplification with universal primers and subsequent quantification by HTS. This synthetic immune system can then be used to calibrate the multiplex PCR assay. Iteratively, the multiplex pool of templates is amplified and sequenced with TCRB V/J-specific primers, and the primer concentrations are adjusted to re-balance PCR amplification. Once the multiplex primer mixture amplifies each V and J template nearly equivalently, residual bias is removed computationally. The parallel procedure for TCRG was described previously in Carlson et al. Nature Communications. 2013; 4:2680.

In some embodiments, the synthetic templates comprise a template composition of general formula (I):

5′-U1-B1-V-B2-X-J-B3-U2-3′ (I)

The constituent template oligonucleotides, of which the template composition is comprised, are diverse with respect to the nucleotide sequences of the individual template oligonucleotides. The individual template oligonucleotides can vary in nucleotide sequence considerably from one another as a function of significant sequence variability among the large number of possible TCR or BCR variable (V) and joining (J) region polynucleotides. Sequences of individual template oligonucleotide species can also vary from one another as a function of sequence differences in U1, U2, B (B1, B2 and B3) and R oligonucleotides that are included in a particular template within the diverse plurality of templates.

In certain embodiments, V is a polynucleotide comprising at least 20, 30, 60, 90, 120, 150, 180, or 210, and not more than 1000, 900, 800, 700, 600 or 500 contiguous nucleotides of an adaptive immune receptor variable (V) region encoding gene sequence, or the complement thereof, and in each of the plurality of template oligonucleotide sequences V comprises a unique oligonucleotide sequence.

In some embodiments, J is a polynucleotide comprising at least 15-30, 31-60, 61-90, 91-120, or 120-150, and not more than 600, 500, 400, 300 or 200 contiguous nucleotides of an adaptive immune receptor joining (J) region encoding gene sequence, or the complement thereof, and in each of the plurality of template oligonucleotide sequences J comprises a unique oligonucleotide sequence.

U1 and U2 can be each either nothing or each comprise an oligonucleotide having, independently, a sequence that is selected from (i) a universal adaptor oligonucleotide sequence, and (ii) a sequencing platform-specific oligonucleotide sequence that is linked to and positioned 5′ to the universal adaptor oligonucleotide sequence.

B1, B2 and B3 can be each either nothing or each comprise an oligonucleotide B that comprises a first and a second oligonucleotide barcode sequence of 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900 or 1000 contiguous nucleotides (including all integer values therebetween), wherein in each of the plurality of template oligonucleotide sequences B comprises a unique oligonucleotide sequence in which (i) the first barcode sequence uniquely identifies the unique V oligonucleotide sequence of the template oligonucleotide and (ii) the second barcode sequence uniquely identifies the unique J oligonucleotide sequence of the template oligonucleotide.

X can be either nothing or comprises a restriction enzyme recognition site that comprises an oligonucleotide sequence that is absent from V, J, U1, U2, B1, B2 and B3.

The template compositions can also include random (R) sequences of length N. Random sequences R can include 2, 3, 4, 5, 6, 7, 8, 9, 10, 11, or more random contiguous nucleotides in each template composition and can be unique to each template composition. There can be one or more R sequences in each synthetic template composition. The random sequences may be inserted in various sections between or within the components in the general formula I (5′-U1-B1-V-B2-X-B3-J-B4-U2-3′) and be of various lengths in size. For example the general formula can be 5′-U1-B1-V-R-B2-X-B3-J-B4-U2-3′ and N can be at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 70, 80, 90, 100, 200, 300, 300, 500 or more contiguous nucleotides. The random sequence can be used to uniquely identify each specific paired V-J combination or to quantify or estimate the number of molecules in a sample.

Methods are used with the template composition for determining non-uniform nucleic acid amplification potential among members of a set of oligonucleotide amplification primers that are capable of amplifying productively rearranged DNA encoding one or a plurality of adaptive immune receptors in a biological sample that comprises DNA from lymphoid cells of a subject. The method can include the steps of: (a) amplifying DNA of a template composition for standardizing amplification efficiency of an oligonucleotide primer set in a multiplex polymerase chain reaction (PCR) that comprises: (i) the template composition (I) described above, wherein each template oligonucleotide in the plurality of template oligonucleotides is present in a substantially equimolar amount; (ii) an oligonucleotide amplification primer set that is capable of amplifying productively rearranged DNA encoding one or a plurality of adaptive immune receptors in a biological sample that comprises DNA from lymphoid cells of a subject.

The primer set can include: (1) in substantially equimolar amounts, a plurality of V-segment oligonucleotide primers that are each independently capable of specifically hybridizing to at least one polynucleotide encoding an adaptive immune receptor V-region polypeptide or to the complement thereof, wherein each V-segment primer comprises a nucleotide sequence of at least 15 contiguous nucleotides that is complementary to at least one functional adaptive immune receptor V region-encoding gene segment and wherein the plurality of V-segment primers specifically hybridize to substantially all functional adaptive immune receptor V region-encoding gene segments that are present in the template composition, and (2) in substantially equimolar amounts, a plurality of J-segment oligonucleotide primers that are each independently capable of specifically hybridizing to at least one polynucleotide encoding an adaptive immune receptor J-region polypeptide or to the complement thereof, wherein each J-segment primer comprises a nucleotide sequence of at least 15 contiguous nucleotides that is complementary to at least one functional adaptive immune receptor J region-encoding gene segment and wherein the plurality of J-segment primers specifically hybridize to substantially all functional adaptive immune receptor J region-encoding gene segments that are present in the template composition.

The V-segment and J-segment oligonucleotide primers are capable of promoting amplification in said multiplex polymerase chain reaction (PCR) of substantially all template oligonucleotides in the template composition to produce a multiplicity of amplified template DNA molecules, said multiplicity of amplified template DNA molecules being sufficient to quantify diversity of the template oligonucleotides in the template composition, and wherein each amplified template DNA molecule in the multiplicity of amplified template DNA molecules is less than 1000, 900, 800, 700, 600, 500, 400, 300, 200, 100, 90, 80 or 70 nucleotides in length.

The method also includes steps of: (b) sequencing all or a sufficient portion of each of said multiplicity of amplified template DNA molecules to determine, for each unique template DNA molecule in said multiplicity of amplified template DNA molecules, (i) a template-specific oligonucleotide DNA sequence and (ii) a relative frequency of occurrence of the template oligonucleotide; and (c) comparing the relative frequency of occurrence for each unique template DNA sequence from said template composition, wherein a non-uniform frequency of occurrence for one or more template DNA sequences indicates non-uniform nucleic acid amplification potential among members of the set of oligonucleotide amplification primers. The amounts for each V-segment and J-segment primer set used in subsequent amplification assays can be adjusted to reduce amplification bias across the primer sets based on the relative frequency of occurrence for each unique template DNA sequence in the template composition.

Further description about bias control compositions and methods are provided in U.S. Provisional Application No. 61/726,489, filed Nov. 14, 2012, U.S. Provisional Application No. 61/644,294, filed on May 8, 2012, U.S. Provisional Application 61/949,069 filed on Mar. 6, 2014, and PCT/US2013/040221, filed on May 8, 2013, PCT/US2013/045994 (WO/2013/188831), filed on Jun. 14, 2013, which are incorporated by reference in their entireties.

### Sequencing

Sequencing may be performed using any of a variety of available high throughput single molecule sequencing machines and systems. Illustrative sequence systems include sequence-by-synthesis systems such as the Illumina Genome Analyzer and associated instruments (Illumina, Inc., San Diego, Calif.), Helicos Genetic Analysis System (Helicos BioSciences Corp., Cambridge, Mass.), Pacific Biosciences PacBio RS (Pacific Biosciences, Menlo Park, Calif.), or other systems having similar capabilities. Sequencing is achieved using a set of sequencing oligonucleotides that hybridize to a defined region within the amplified DNA molecules. The sequencing oligonucleotides are designed such that the V- and J-encoding gene segments can be uniquely identified by the sequences that are generated, based on the present disclosure and in view of known adaptive immune receptor gene sequences that appear in publicly available databases. Exemplary sequencing oligonucleotides are described in Robins et al., 2009 Blood 114, 4099; Robins et al., 2010 Sci. Translat. Med. 2:47ra64; Robins et al., 2011 J. Immunol. Meth. doi:10.1016/j.jim.2011.09. 001; Sherwood et al. 2011 Sci. Translat. Med. 3:90ra61; U.S. Ser. No. 13/217,126, U.S. Ser. No. 12/794,507, WO/2010/151416, WO/2011/106738 (PCT/US2011/026373), WO2012/027503 (PCT/U52011/049012), U.S. Ser. No. 61/550,311, U.S. Ser. No. 61/569,118, and PCT/US2013/045994 (WO/2013/188831), filed on Jun. 14, 2013, which are incorporated by reference in their entireties.

Any technique for sequencing nucleic acid known to those skilled in the art can be used in the methods of the provided invention. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of the separated molecules has more recently been demonstrated by sequential or single extension reactions using polymerases or ligases as well as by single or sequential differential hybridizations with libraries of probes. These reactions have been performed on many clonal sequences in parallel including demonstrations in current commercial applications of over 100 million sequences in parallel. These sequencing approaches can thus be used to study the repertoire of T-cell receptor (TCR) and/or B-cell receptor (BCR).

The sequencing technique used in the methods of the invention can generate least 1000 reads per run, at least 10,000 reads per run, at least 100,000 reads per run, at least 500,000 reads per run, or at least 1,000,000 reads per run. The sequencing technique used in the methods of the invention can generate about 30 bp, about 40 bp, about 50 bp, about 60 bp, about 70 bp, about 80 bp, about 90 bp, about 100 bp, about 110, about 120 bp, about 150 bp, about 200 bp, about 250 bp, about 300 bp, about 350 bp, about 400 bp, about 450 bp, about 500 bp, about 550 bp, or about 600 bp per read. The sequencing technique used in the methods of the invention can generate at least 30, 40, 50, 60, 70, 80, 90, 100, 110, 120, 150, 200, 250, 300, 350, 400, 450, 500, 550, or 600 bp per read.

Example sequencing methods include, but are not limited to, true single molecule sequencing (tSMS), 454 sequencing (Roche), SOLiD sequencing (Applied Biosystems), SOLEXA sequencing (Illumina), SMRT Sequencing (Pacific Biosciences), nanopore sequencing, chemical-sensitive field effect transistor array sequencing, or sequencing by electron microscope, or other high throughput sequencing methods known to those of skill in the art.

In some embodiments, bias-controlled V segment and J segment gene primers are used to amplify rearranged V(D)J segments to produce a plurality of amplicons for high throughput sequencing at ˜20× coverage. Coverage is the number of copies sequenced of each synthetic template.

### Processing Sequence Data

As presently disclosed, there are also provided methods for analyzing the sequences of the diverse pool of uniquely rearranged CDR3-encoding regions that are generated using the compositions and methods that are described herein. As described above, amplification bias can be corrected using bias control synthetic templates.

Corrections can also be made for PCR errors and for estimating true distribution of specific clonotypes (e.g., a TCR or IG having a uniquely rearranged CDR3 sequence) in blood or in a sample derived from other peripheral tissue or bodily fluid.

In some embodiments, the sequenced reads are filtered for those including CDR3 sequences. Sequencer data processing involves a series of steps to remove errors in the primary sequence of each read, and to compress the data. A complexity filter removes approximately 20% of the sequences that are misreads from the sequencer. Then, sequences were required to have a minimum of a six base match to both one of the TCR or IG J-regions and one of the TCR or IG V-regions. Applying the filter to the control lane containing phage sequence, on average only one sequence in 7-8 million passed these steps. Finally, a nearest neighbor algorithm is used to collapse the data into unique sequences by merging closely related sequences, in order to remove both PCR error and sequencing error.

Analyzing the data, the ratio of sequences in the PCR product are derived working backward from the sequence data before estimating the true distribution of clonotypes (e.g., unique clonal sequences) in the blood. For each sequence observed a given number of times in the data herein, the probability that that sequence was sampled from a particular size PCR pool is estimated. Because the CDR3 regions sequenced are sampled randomly from a massive pool of PCR products, the number of observations for each sequence are drawn from Poisson distributions. The Poisson parameters are quantized according to the number of T cell genomes that provided the template for PCR. A simple Poisson mixture model both estimates these parameters and places a pairwise probability for each sequence being drawn from each distribution. This is an expectation maximization method, which reconstructs the abundances of each sequence that was drawn from the blood.

In some embodiments, to estimate the total number of unique adaptive immune receptor CDR3 sequences that are present in a sample, a computational approach employing the “unseen species” formula may be employed (Efron and Thisted, 1976 Biometrika 63, 435-447). This approach estimates the number of unique species (e.g., unique adaptive immune receptor sequences) in a large, complex population (e.g., a population of adaptive immune cells such as T cells or B cells), based on the number of unique species observed in a random, finite sample from a population (Fisher et al., 1943 J. Anim. Ecol. 12:42-58; Ionita-Laza et al., 2009 Proc. Nat. Acad. Sci. USA 106:5008). The method employs an expression that predicts the number of “new” species that would be observed if a second random, finite and identically sized sample from the same population were to be analyzed. “Unseen” species refers to the number of new adaptive immune receptor sequences that would be detected if the steps of amplifying adaptive immune receptor-encoding sequences in a sample and determining the frequency of occurrence of each unique sequence in the sample were repeated an infinite number of times. By way of non-limiting theory, it is operationally assumed for purposes of these estimates that adaptive immune cells (e.g., T cells, B cells) circulate freely in the anatomical compartment of the subject that is the source of the sample from which diversity is being estimated (e.g., blood, lymph, etc.).

To apply this formula, unique adaptive immune receptors (e.g., TCRβ, TCRα, TCRγ, TCRδ, IgH) clonotypes takes the place of species. The mathematical solution provides that for S, the total number of adaptive immune receptors having unique sequences (e.g., TCRβ, TCRγ, IgH “species” or clonotypes, which may in certain embodiments be unique CDR3 sequences), a sequencing experiment observes xₛ copies of sequence s. For all of the unobserved clonotypes, xₛ equals 0, and each TCR or Ig clonotype is “captured” in the course of obtaining a random sample (e.g., a blood draw) according to a Poisson process with parameter λₛ. The number of T or B cell genomes sequenced in the first measurement is defined as 1, and the number of T or B cell genomes sequenced in the second measurement is defined as t.

Because there are a large number of unique sequences, an integral is used instead of a sum. If G(λ) is the empirical distribution function of the parameters λ₁, . . . , λ_{S}, and nₓ is the number of clonotypes (e.g., unique TCR or Ig sequences, or unique CDR3 sequences) observed exactly x times, then the total number of clonotypes, i.e., the measurement of diversity E, is given by the following formula (I):

$$E ( {n}_{x} ) = S {\int }_{0}^{∞} ( \frac{{e}^{- λ} {λ}^{x}}{x !} ) d G ( λ ) . \qquad ( I )$$

Accordingly, formula (I) may be used to estimate the total diversity of species in the entire source from which the identically sized samples are taken. Without wishing to be bound by theory, the principle is that the sampled number of clonotypes in a sample of any given size contains sufficient information to estimate the underlying distribution of clonotypes in the whole source. The value for Δ(t), the number of new clonotypes observed in a second measurement, may be determined, preferably using the following equation (II):

$$Δ ( t ) = {\sum }_{x} {E ( {n}_{x} )}_{msmt 1 + msmt 2} - {\sum }_{x} {E ( {n}_{x} )}_{msmt 1} = S {\int }_{0}^{∞} {e}^{- λ} ( 1 - {e}^{- λ t} ) d G ( λ ) \qquad ( II )$$

in which msmt1 and msmt2 are the number of clonotypes from measurements 1 and 2, respectively. Taylor expansion of 1-e^{-λt} and substitution into the expression for Δ(t) yields:

Δ(t)=E(x₁)t−E(x₂)t²+E(x₃)t³− (III)

which can be approximated by replacing the expectations (E(nₓ)) with the actual numbers sequences observed exactly x times in the first sample measurement. The expression for Δ(t) oscillates widely as t goes to infinity, so Δ(t) is regularized to produce a lower bound for Δ(∞), for example, using the Euler transformation (Efron et al., 1976 Biometrika 63:435).

In one example, using the numbers observed in a first measurement of TCRβ sequence diversity in a blood sample, this formula (II) predicted that 1.6*10⁵ new unique sequences should be observed in a second measurement. The actual value of the second measurement was 1.8*10⁵ new TCRβ sequences, which suggested according to non-limiting theory that the prediction provided a valid lower bound on total TCRβ sequence diversity in the subject from whom the sample was drawn.

Additional description about the unseen species model and processing sequence data are described in Robins et al., 2009 Blood 114, 4099; Robins et al., 2010 Sci. Translat. Med. 2:47ra64; Robins et al., 2011 J. Immunol. Meth. doi:10.1016/j.jim.2011.09. 001; Sherwood et al. 2011 Sci. Translat. Med. 3:90ra61; U.S. Ser. No. 13/217,126, U.S. Ser. No. 12/794,507, WO/2010/151416, WO/2011/106738 (PCT/US2011/026373), WO2012/027503 (PCT/US2011/049012), U.S. Ser. No. 61/550,311, and U.S. Ser. No. 61/569,118, which are incorporated by reference in their entireties.

In certain embodiments, after correcting sequencing errors via a clustering algorithm, CDR3 segments are annotated according to the International ImMunoGeneTics collaboration. See Lefranc, M.-P., Giudicelli, V., Ginestoux, C., Jabado-Michaloud, J., Folch, G., Bellahcene, F., Wu, Y., Gemrot, E., Brochet, X., Lane, J., Regnier, L., Ehrenmann, F., Lefranc, G. and Duroux, P. IMGT®, the International ImMunoGeneTics Information system®. Nucl. Acids Res, 37, D1006-D1012 (2009); doi:10.1093/nar/gkn838. PMID: 18978023; Lefranc, M.-P., IMGT, the International ImMunoGeneTics Information System. Cold Spring Harb Protoc. 2011 Jun. 1. 2011(6). pii: pdb.top115. doi: 10.1101/pdb.top115. PMID: 21632786.

PCR Template Abundance Estimation

In order to estimate the average read coverage per input template in a multiplex PCR and high throughput sequencing approach, a set of unique types of synthetic templates can be employed. For example, the set of unique types of synthetic templates can comprise every combination of Vβ and Jβ gene segments (25) for TCRB and every combination of Vγ and Jγ gene segments (approximately 75) for TCRG. These molecules are included in each PCR reaction at very low concentration (e.g., a limiting dilution), such that most unique types of synthetic template are not observed in the sequencing output. Thus, based on the limiting dilution calculation, there is a known number of synthetic templates in the starting material.

A multiplex amplification reaction and HTS assay are performed using the synthetic templates and biological templates. Using the known concentration of the synthetic template pool, a relationship is simulated between the number of observed unique synthetic molecules (e.g., the number of output sequence reads for synthetic templates) and the total number of synthetic templates added to reaction (e.g., number of starting synthetic templates). This relationship is very nearly one-to-one at the low concentrations that are used. These synthetic molecules allow for calculation for each PCR reaction, the mean number of sequencing reads obtained per molecule of PCR template. An amplification factor is determined for each input template based on the ratio of the number of input synthetic templates to the number of output sequencing reads per synthetic template. This amplification factor is then used to estimate the number of T cells that are unique clones (T cells each having unique TCR rearrangement) in the input material.

Clonal Detection

A putative cancer clone is defined by sequence abundance. A clone can have either one or two rearranged alleles. In TCRG, the majority of clones, both TCRG alleles are rearranged. For TCRB, a minority of clones has both alleles rearranged. For consistency, a clone's abundance was defined by summing the abundance of the top two alleles for TCRG and the top single allele for TCRB. For TCRG, the method is robust for the cases with only a single rearrangement, as the second largest allele, which is present in a non-malignant cell, is relatively low.

The percent of T cells consisting of the cancer clone is determined by dividing the abundance of the cancer clone (number of sequence reads for a unique TCR sequence) by the total number of T cell reads. For TCRG, the number of sequence reads for the cancer clone was divided by two because most cells have two rearrangements. The fraction of total nucleated cells is determined by multiplying this number by the fraction of T cells in the samples.

In one embodiment, the method for determining the fraction of a T cell clone in the total nucleated cells can include the following steps:
(1) Determine an amplification factor for a primer set for a particular unique rearranged TCR sequence. A set of synthetic templates is used to measure amplification bias of a set of amplification primers. For example, the amount of synthetic templates added to the PCR assay is known based on limiting dilution. The synthetic templates are added to each PCR reaction at very low concentration (e.g., a limiting dilution), such that most unique types of synthetic template are not observed in the sequencing output. A multiplex PCR and high throughput sequencing is performed using the synthetic templates, biological templates, and a plurality of V-segment and J-segment primers. The number of sequence reads for each unique T cell sequence and number of sequence reads for each synthetic template are quantified. The amplification factor can be determined by dividing the number of output sequence reads for the synthetic template to the number of input molecules for a synthetic template. For example, if there are 10 input molecules for a particular synthetic template, and an output of 100 sequence reads for that synthetic template, the amplification factor is 100/10=10.
(2) Determine total number of nucleated cells in sample. The amount of DNA added to a PCR is known. It is estimated that each cell has approximately 6.0 pg DNA. The total number of nucleated cells can be estimated by dividing the total amount of DNA/(6.0 picograms/genome).
(3) Determine the top T cell clone in a sample based on frequency of occurrence of sequence reads. For each unique TCR sequence, determine the number of sequence reads in the sample and identify the unique TCR sequence with the highest frequency of occurrence (top T cell clone).
(4) Determine the number of T cells with the same unique TCR sequence. Divide the number of sequence reads for a T cell clone by the amplification factor to get the number of T cells with the same unique TCR sequence.
(5) Determine the fraction of the T cell clone in the total number of nucleated cells. Divide the number of T cells of the same T cell clone (from 4) by the total number of nucleated cells (from 2).

One can also determine the fraction of the T cell clone in the total number of T cells. The total number of T cells is determined by dividing the total number of sequence reads from the HTS by the amplification factor (in 1).

The fraction of a top T cell clone in a total number of nucleated cells or the fraction of a top T cell clone in a total number of T cells can be represented as a log₁₀ norm.

This method can be used similarly for B cells and determining the fraction of a B cell clone in the total nucleated cells.

Cryosection Immunostaining

CTCL skin samples can be embedded in optimal cutting temperature (OCT), frozen, and stored at −80° C. until use. Cryosections can be cut, air dried, fixed for 5 min in acetone, rehydrated in PBS, and blocked with 20 μg/ml of human IgG (Jackson ImmunoResearch Laboratories) for 15 min at room temperature. Sections can be incubated with directly conjugated anti-TCR Vβ antibody for 30 min, and then rinsed three times in PBS/1% BSA for 5 min. Sections can be mounted using Prolong Gold Antifade with DAPI (Life Technologies) and examined immediately by immunofluorescence microscopy. Sections can be photographed using a microscope (Eclipse 6600; Nikon) equipped with a 40×/0.75 objective lens (Plan Fluor; Nikon). Images can be captured with a camera (SPOT RT model 2.3.1; Diagnostic Instruments) and acquired with SPOT 4.0.9 software (Diagnostic Instruments).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Characterization of T Cell Clonality and Malignancy Detection

In this example, samples of blood, bone marrow, skin, and lymph node and tonsil tissue from 99 patients were obtained from the University of Washington (Department of Laboratory Medicine). 96 of these patients had a presumed T-cell malignancy, and the remaining 3 patients had been diagnosed with a myeloproliferative disorder. Control samples were also obtained from individuals without evidence of leukemia, lymphoma, or other cancer (14 lymphoid tissue samples, 7 blood samples, 8 bone marrow samples), from the University of Washington.

Genomic DNA was prepared using a Qiagen DNA extraction kit from the samples of peripheral blood mononuclear cells (PBMCs), bone marrow, frozen or formalin-fixed paraffin-embedded (FFPE) 4 mm skin punch biopsies, or FFPE lymph node samples. DNA corresponding to 60,000-200,000 genomes was used for index clone identification. Approximately one million total cells were studied for determination of residual disease.

A multiplex PCR system was used to amplify rearranged TCRB or TCRG loci as previously described in U.S. Ser. No. 12/794,507 and U.S. Ser. No. 13/217,126, which is each incorporated by reference in its entirety. A set of forward primers (each specific to a functional TCRB or TCRG V gene segment) and a set of reverse primers (each specific to a TCRB or TCRG J gene segment) were used in solid-phase PCR (Illumina HiSeq platform) to generate amplicons that cover the entire complementarity determining region (CDR3). The CDR3 encoding regions of TCRB and TCRG genes were sequenced for all 99 specimens by high-throughput sequencing (HTS) using an Illumina GA2 platform (Illumina, San Diego, Calif.) as previously described (see Robins et al., Blood, 114(19):4099-4107, 2009; and Robins et al., Sci Transl Med. 3(90):90ra61, 2011). The samples were used to identify unique rearranged TCR CDR3-encoding DNA sequences, and the samples were assessed for the frequency of each rearranged TCR CDR3-encoding DNA sequence as a percentage of the total.

The total number of observed rearranged amplicon sequences in each sample was counted. The frequency of occurrence of each unique rearranged amplicon sequence was also calculated by dividing the number of counts of each unique rearranged amplicon sequence by the total number of rearranged amplicon sequences in the sample. To obtain the number of copies of a particular unique rearranged amplicon sequence in a sample, the frequency of occurrence of that amplicon sequence was divided by an amplification factor as described above. Distinct TCRB or TCRG CDR3 amplicon sequences were plotted on the basis of their frequency of appearance to generate the TCR “repertoire” (the number of counts corresponding to a specific TCRB or TCRG sequence/amplicon, divided by the total number of TCRB or TCRG sequences generated from the given sample).

FIG. 1 illustrates a box and whiskers plot indicating the distribution of the most abundant TCR clone in the healthy controls by tissue type. The portion indicating 2^{nd} QT shows values representing frequencies between the 25^{th} and 50^{th} percentile. The portion indicating 3^{rd} QT shows values representing frequencies between the 51^{st} and 75^{th} percentile. Table 1 shows the averages and standard deviations of the highest copy TCR amplicons in the control samples. An atypical amplicon, or an amplicon exhibiting a frequency of occurrence at a statistically significant difference from the average reference frequency, was marked as potentially malignant. In this case, an atypical amplicon was determined to be one occurring at a frequency of at least 7 standard deviations above average. Thus, for example, for Lymph/tonsil in Table 1, an atypical clone was determined to be 3.32%, or (7×0.38%)+0.64%.

TABLE 1
Defining abnormal amplicons based on distribution of
TCR repertoire in normal samples

| | | | Standard | Atypical clone (at least |
|---|---|---|---|---|
| | N | Average | deviation | 7 standard deviations |
| | samples | (%) | (%) | above average) (%) |
| Lymph/tonsil | 14 | 0.64 | 0.38 | 3.32 |
| Blood | 7 | 1.73 | 1.73 | 13.83 |
| Bone marrow | 8 | 1.52 | 1.50 | 12.02 |

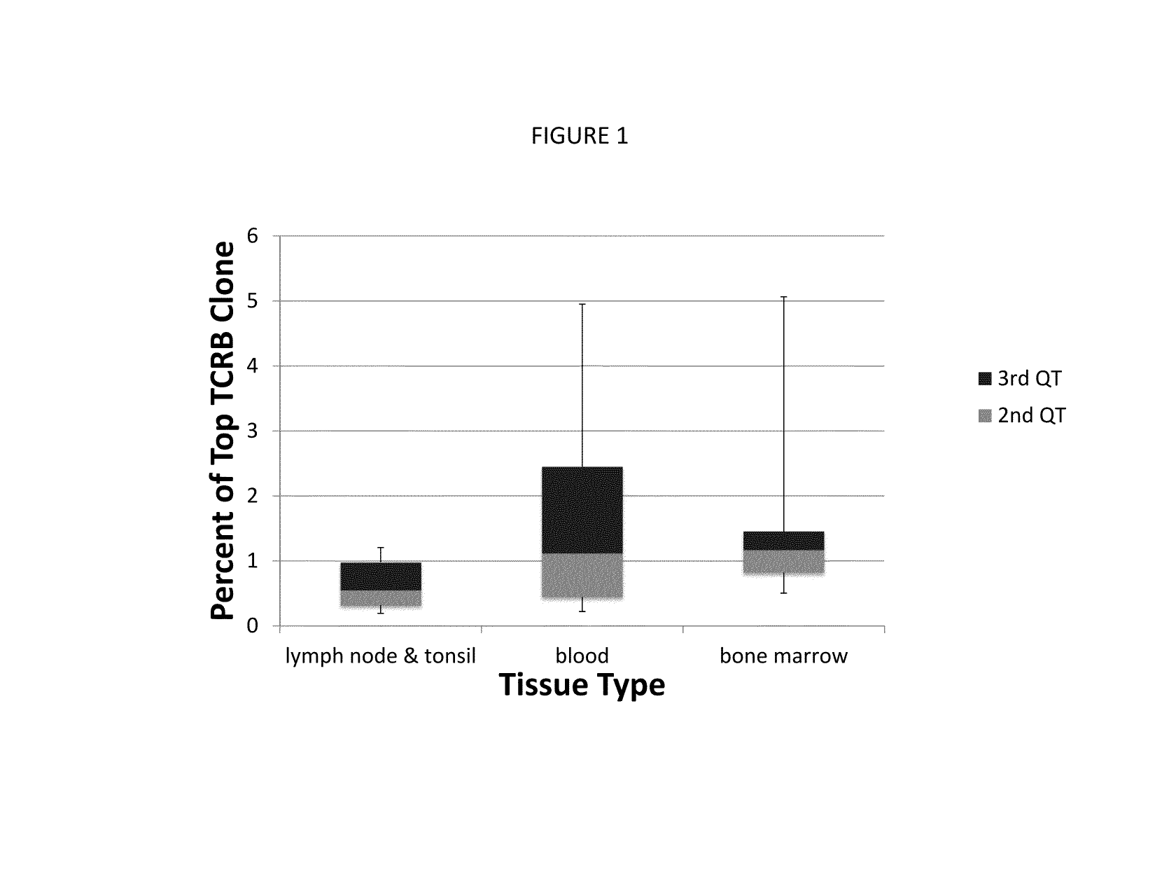

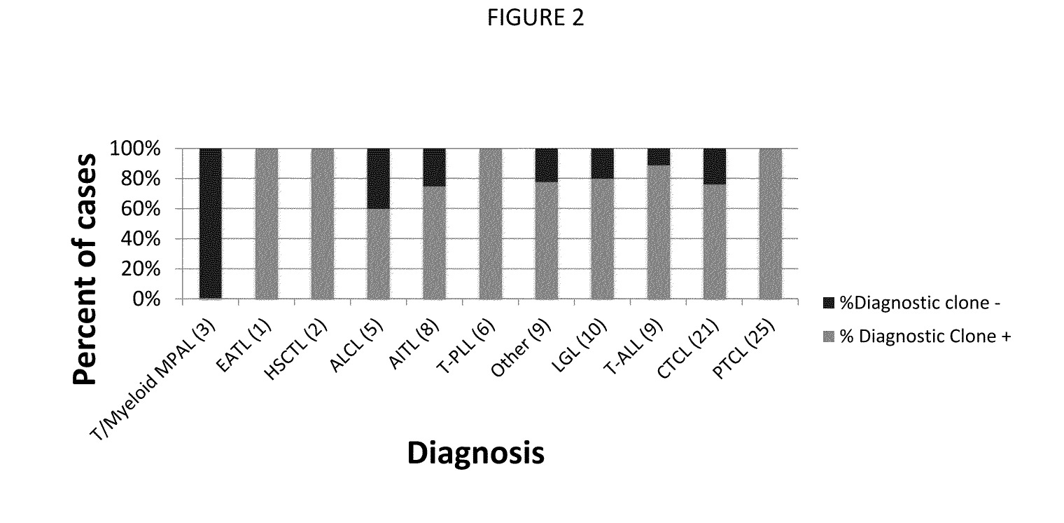

FIG. 2 shows the identification of diagnostic clones identified as malignant in various samples (a clone is a unique rearranged amplicon sequence). For a selected unique rearranged amplicon sequence, the percentage of the number of the unique rearranged amplicon sequence in the total number of observed rearranged amplicon sequences was determined in each sample. The figure shows the samples categorized by diagnosis of the patient: 3 samples of MPAL (mixed phonotypic acute leukemia), 1 sample of EATL (enteropathy associated T lymphoma), 2 samples of HSCTL (hepatosplenic T-cell lymphoma), 5 samples of ALCL (anaplastic large cell lymphoma), 8 samples of AITL (angioimmunoblastic T lymphoma), 6 samples of T-PLL (T prolymphocytic leukemia), 9 samples of other, 10 samples of LGL (large granular lymphoma), 9 samples of T-ALL (T acute lymphoblastic leukemia), 21 samples of CTCL (cutaneous T cell lymphoma), and 25 samples of PTCL (peripheral T cell lymphoma). In this example, the diagnostic clone was defined in this case as malignant if it had an average frequency that was at least 7 standard deviations above the average of the selected unique rearranged amplicon frequency in a control. This was the calculation used for determining a malignant clone, but others can be used, as described herein. The figure indicates that even using this stringent criterion for defining an index clone and even in the context of relatively subjective and nonstandardized histopathological diagnoses, the multiplex PCR and HTS (high-throughput sequencing) approach identifies major clones in the majority of T-cell malignancies.

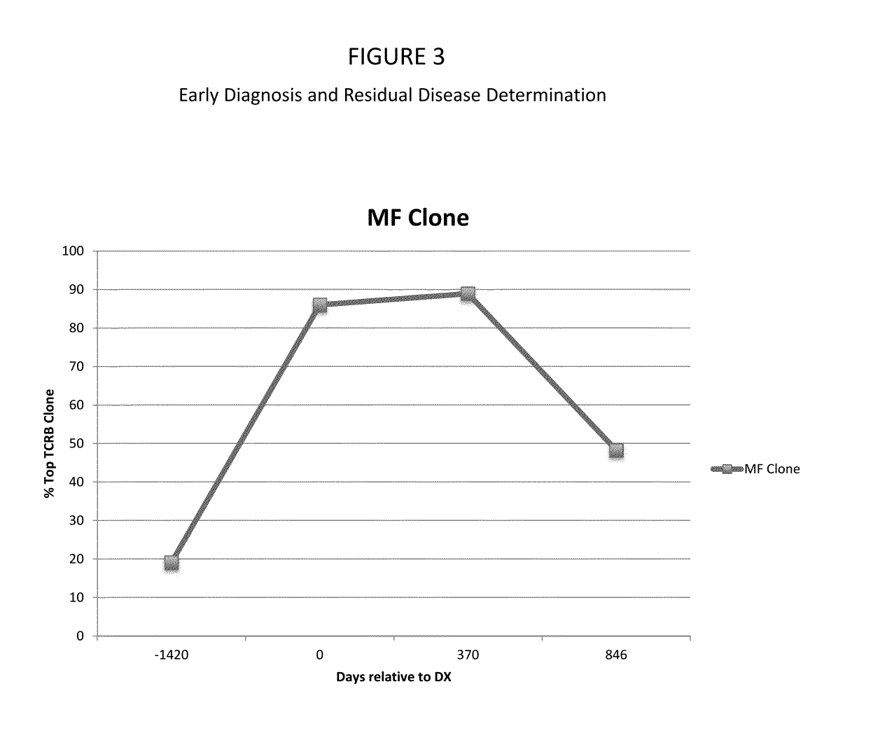

As shown in FIG. 3, biopsy samples were taken from a patient at different time points for over six years, and the percentage of the unique rearranged amplicon sequence present at the highest frequency of occurrence was measured over this period of time. The percentage of the top T cell clone was consistent with a T-cell hyperproliferation or expansion which, given other clinical signs and symptoms, was indicative of Mycosis fungoides. On Day −1420, the patient had found a suspect skin lesion (the x-axis of FIG. 3 shows the number of days relative to a Mycosis fungoides, or MF, diagnosis). However, the biopsy sample obtained on day −1420 indicated a top TCRB clone, but did not indicate a very high frequency of occurrence for that clone (20%). On Day 0, the biopsy sample showed that the frequency of occurrence of the top TCRB clone had increased greatly, up to around 85%. The HTS assay was used to sequence the clones in the various samples, and the malignant clone was identified over four years before the clinical diagnosis was made. Furthermore, the same clone was shown to be further expanded at the time of the clinical diagnosis. Therefore, methods of this invention can be used for early diagnosis of patients.

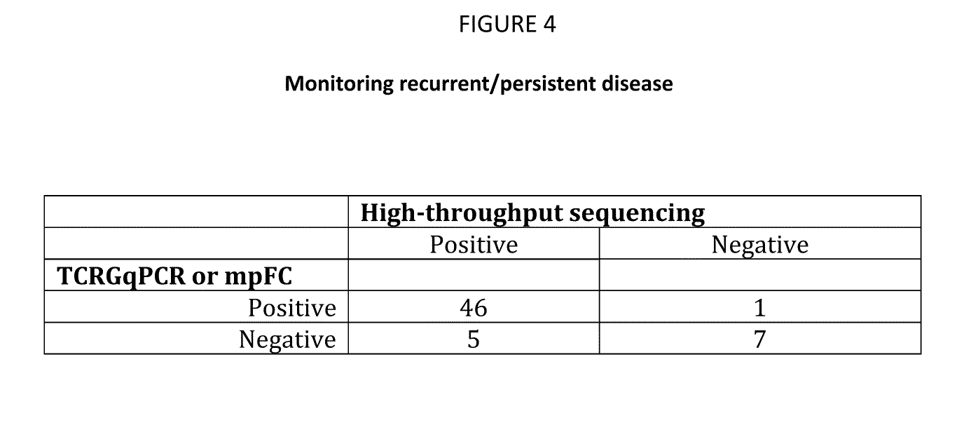

FIG. 4 illustrates identification of a trackable clone (positive or negative) using (1) high throughput sequencing (HTS) or (2) TCRG quantitative PCR or multiparametric flow cytometry (mpFC). For the samples for which both HTS and TCRG qPCR or mpFC identified a trackable clone, HTS was able to track residual disease in all but one of the samples tracked by the other methods, and was additionally able to detect residual disease in five samples that were missed by the other methods. This shows the sensitivity and accuracy of the HTS method.

This method can be used similarly for B cells and identifying a trackable B-cell clone (positive or negative) using (1) high throughput sequencing (HTS).

### Example 2

### Cutaneous T-Cell Lymphoma Detection

The methods of the invention were used to detect CTCL using HTS. 47 individuals were included in this study. 37 individuals had been previously diagnosed with CTCL or lymphoproliferative disease. 6 biopsies were taken from the skin of “normal” control individuals, 2 biopsies were taken from individuals having a suspect lesion, 1 biopsy was taken from an individual with a benign inflammatory infiltrate, and 1 biopsy was taken from an individual originally having a “diagnostic dilemma” and subsequently being diagnosed with eczematous hypersensitivity reaction.

Genomic DNA was prepared as described in Example 1. Multiplex PCR and HTS were also performed as described in Example 1.

The total number of observed rearranged CDR3 sequences in each sample was counted. The frequency of occurrence of each unique rearranged CDR3 sequence was calculated by dividing the number of counts of each unique rearranged CDR3 sequence by the total number of rearranged CDR3 sequences in the sample. To obtain the number of copies of a particular unique rearranged amplicon sequence in a sample, the frequency of occurrence of that amplicon sequence was divided by an amplification factor as described above. Distinct TCRB or TCRG CDR3 amplicon sequences were plotted on the basis of their frequency of occurrence to generate the TCR “repertoire” (the number of counts corresponding to a specific TCRB or TCRG sequence/amplicon, divided by the total number of TCRB or TCRG sequences observed from the given sample).

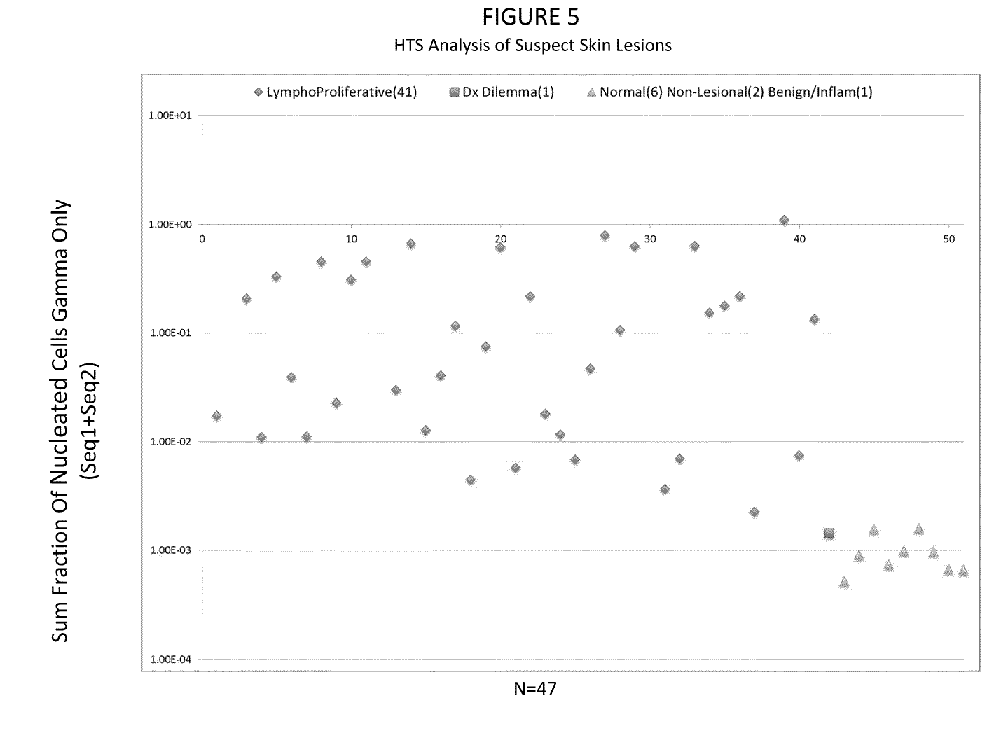

The two unique rearranged amplicon sequences with the highest frequencies of occurrence were selected. For each sample, the two frequencies of occurrence were summed and then divided by the total number of nucleated cells (or genomes) in the corresponding sample. FIG. 5 illustrates these frequencies (sum fraction of nucleated cells) for each sample. The graph shows the samples known to be lymphproliferative, normal, and unknown (“Dx Dilemma”). The graph illustrates that each and every one of the lymphoproliferative samples have a higher sum fraction of the two frequencies of occurrence over the total number of nucleated cells than the normal/benign samples.

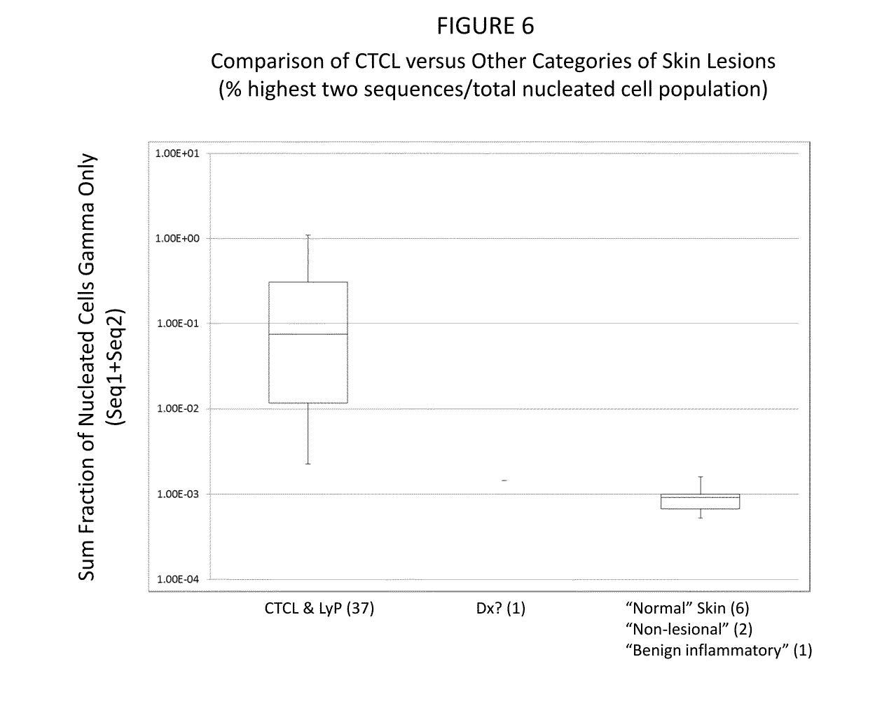

FIG. 6 shows a comparison of samples diagnosed with CTCL versus other skin lesion categories. The box and whiskers plot illustrates the distribution of the two unique rearranged amplicon sequences with the highest frequencies of occurrence over the total nucleated cell population. This study was accomplished in a “blinded” fashion in which the assay was performed without knowledge of the clinical diagnosis. The results demonstrate that the assay is a robust discriminator between malignant and non-malignant skin samples, even those that have benign lymphoid infiltrates. Therefore, this assay can inform in the context of all of the signs, symptoms, and other diagnostic tests from a given patient whether that patient suffers from CTCL or not.

This method can also be used for B cell lymphoid neoplasms and other lymphoid proliferative disorders.

### Example 3

### Identification of Expanded T Cell Clones Using High Throughput TCR CDR3 Region Sequencing and Discrimination of CTCL from Benign Inflammatory Skin Disorders

Materials and Methods

Skin and Blood Samples

The protocols of the studies in the examples were performed in accordance with the Declaration of Helsinki, and were approved by the Institutional Review Board of the Partners Human Research Committee (Partners Research Management) and the Dana Farber Cancer Institute Skin from healthy patients was obtained from patients undergoing cosmetic surgery procedures. Blood and lesional skin from patients with CTCL were obtained from patients seen at the Dana-Farber/Brigham and Women's Cancer Center Cutaneous Lymphoma Program. L-CTCL and MF patients described in this study met the WHO-EORTC criteria for CTCL. Lesional skin from patients with psoriasis and eczematous dermatitis were obtained from patients seen at the Brigham and Women's Hospital or at Rockefeller University.

DNA Isolation from Skin

DNA was isolated from frozen, OCT embedded or formalin fixed paraffin embedded (FFPE) skin samples as follows. For OCT embedded tissue samples, 30 cryosections of 10 μm thickness were cut and DNA extraction was carried out using the QIAamp DNA Mini Kit (Qiagen) kit as per manufacturer's instructions with overnight tissue digestion. This method generated 155-3730 ng DNA per sample. For FFPE samples, DNA was extracted from two 40 μm scrolls using the QIAamp DNA Mini Kit as per manufacturer's instructions with the following modifications. Paraffin was removed from tissue scrolls by two rounds of xylene extraction followed by two ethanol washes prior to overnight tissue digestion. Extra proteinase K was added after overnight digestion if visible tissue still remained. This method generated 272-3656 ng DNA per sample.

HTS Analyses—Immunosequencing

For each sample, DNA was extracted skin biopsies, then TCRβ CDR3 and TCRG CDR3 regions were amplified and sequenced using the methods described herein (ImmunoSEQ™ (Adaptive Biotechnologies, Seattle, Wash.)) from 400 ngs of DNA template. Bias-controlled V and J gene primers are used to amplify rearranged V(D)J segments for high throughput sequencing at ˜20× coverage. After correcting sequencing errors via a clustering algorithm, CDR3 segments were annotated according to the International ImMunoGeneTics collaboration. See Lefranc, M.-P., Giudicelli, V., Ginestoux, C., Jabado-Michaloud, J., Folch, G., Bellahcene, F., Wu, Y., Gemrot, E., Brochet, X., Lane, J., Regnier, L., Ehrenmann, F., Lefranc, G. and Duroux, P. IMGT®, the International ImMunoGeneTics Information system®. Nucl. Acids Res, 37, D1006-D1012 (2009); doi:10.1093/nar/gkn838. PMID: 18978023; Lefranc, M.-P., IMGT, the International ImMunoGeneTics Information System. Cold Spring Harb Protoc. 2011 Jun. 1. 2011(6). pii: pdb.top115. doi: 10.1101/pdb.top115. PMID: 21632786.

Controlling Bias in a Multiplex PCR

Since accurate quantification of lymphoblast clones for MRD detection is critical, an approach to ensure minimal bias in multiplex PCR, as described above was developed. See Carlson C S, Emerson R O, Sherwood A M, Desmarais C, Chung M-W, Parsons J M, et al. Using synthetic templates to design an unbiased multiplex PCR assay. Nature Communications. 2013; 4:2680. Briefly, each potential VDJ rearrangement of the TCRB locus contains one of thirteen J segments, one of 2 D segments and one of 52 V segments, many of which have disparate nucleotide sequences. In order to amplify all possible VDJ combinations, used a single tube, multiplex PCR assay with 45 V forward and 13 J reverse primers was used. To remove potential PCR bias, every possible V-J pair was chemically synthesized as a template with specific barcodes. These templates were engineered so as to be recognizable as non-biologic and have universal 3′ and 5′ ends to permit amplification with universal primers and subsequent quantification by HTS. This synthetic immune system can then be used to calibrate the multiplex PCR assay. Iteratively, the multiplex pool of templates is amplified and sequenced with our TCRB V/J-specific primers, and the primer concentrations are adjusted to re-balance PCR amplification once the multiplex primer mixture amplifies each V and J template nearly equivalently, residual bias is removed computationally. The parallel procedure for TCRG was described previously in Carlson C S, Emerson R O, Sherwood A M, Desmarais C, Chung M-W, Parsons J M, et al. Using synthetic templates to design an unbiased multiplex PCR assay. Nature Communications. 2013; 4:2680.

PCR Template Abundance Estimation

In order to estimate the average read coverage per input template in our multiplex PCR and sequencing approach a set of approximately 850 unique types of synthetic TCR analog, comprising each combination of Vβ and Jβ gene segments (25) for TCRB and approximately 75 for TCRG was employed. These molecules were included in each PCR reaction at very low concentration so that most unique types of synthetic template were not observed in the sequencing output. Using the known concentration of the synthetic template pool, the relationship between the number of observed unique synthetic molecules and the total number of synthetic molecules added to reaction (this is very nearly one-to-one at the low concentrations we employed) was simulated. These molecules then allowed for the calculation of the mean number of sequencing reads obtained per molecule of PCR template (the amplification factor) for each PCR reaction, and thus to estimate the number of T cells in the input material bearing each unique TCR rearrangement.

Clonal Detection

The putative cancer clone is defined by sequence abundance. A clone can have either one or two rearranged alleles. For the majority of clones, both TCRG alleles are rearranged and for TCRB a minority has both alleles rearranged. For consistency, a clone's abundance was defined by summing the abundance of the top two alleles for TCRG and the top single allele for TCRB. For TCRG the method is robust for the cases with only a single rearrangement, as the second largest allele which is present in a non-malignant cell is relatively low. The percent of T cells consisting of the cancer clone is determined by dividing the abundance of the cancer clone (number of reads) by the total number of T cell reads. For TCRG, this calculation was further divided by two because most cells have two rearrangements. The fraction of total nucleated cells is determined by multiplying this number by the fraction of T cells in the samples as described above.

Statistical Analyses

Results in different patient sample groups were compared using one-way ANOVA analyses using the non-parametric Kruskal-Wallis test and the Dunn's multiple comparison post-test.

Cryosection Immunostaining

CTCL skin samples were embedded in OCT, frozen, and stored at −80° C. until use. 5-μm cryosections were cut, air dried, fixed for 5 min in acetone, rehydrated in PBS, and blocked with 20 μg/ml of human IgG (Jackson ImmunoResearch Laboratories) for 15 min at room temperature. Sections were incubated with directly conjugated anti-TCR Vβ antibody for 30 min, and then rinsed three times in PBS/1% BSA for 5 min. Sections were mounted using Prolong Gold Antifade with DAPI (Life Technologies) and examined immediately by immunofluorescence microscopy. Sections were photographed using a microscope (Eclipse 6600; Nikon) equipped with a 40×/0.75 objective lens (Plan Fluor; Nikon). Images were captured with a camera (SPOT RT model 2.3.1; Diagnostic Instruments) and were acquired with SPOT 4.0.9 software (Diagnostic Instruments).

Identification of Expanded T Cell Clones Using High Throughput TCR CDR3 Region Sequencing and Discrimination of CTCL from Benign Inflammatory Skin Disorders

Expanded T cell clones were identified using multiplex PCR and high throughput sequencing methods described herein. DNA from 46 CTCL skin lesions, lesional skin from 23 patients with psoriasis, 11 patients with eczematous dermatitis and the skin of 6 healthy donors were analyzed by HTS for both the TCR Vγ and TCR Vβ genes. HTS was used to determine to provide earlier and more accurate diagnosis of CTCL.

Clinical information regarding the CTCL patients studied is provided in Table 2.

TABLE 2
Stages of CTCL patient samples

| | Stage | Stage | Stage | Stage | Stage | Stage | |
|---|---|---|---|---|---|---|---|
| | IA | IB | IIA | IIB | III | IV | Total |
| Blood | 0 | 4 | 0 | 2 | 0 | 2 | 8 |
| Skin | 11 | 19 | 5 | 8 | 2 | 5 | 50 |

Clonality values were calculated from entropy of the TCR V-β0 CDR3 frequency distribution, and then normalized by log (# unique TCR CDR3). These values range from 0 (polyclonal distribution) to 1 (monoclonal distribution). As expected, the aggregate clonality of T cells in CTCL skin lesions, increased with increasing stage of disease. FIG. 1A shows that clonality of lesional skin T cells increased with advanced stage of CTCL. The mean and SEM of clonality scores of 8 patients with Stage IA, 18 patients with stage IB, 5 patients with stage IIA, 7 patients with stage IIB, 2 patients with stage III, and 5 patients with stage IV disease are shown.

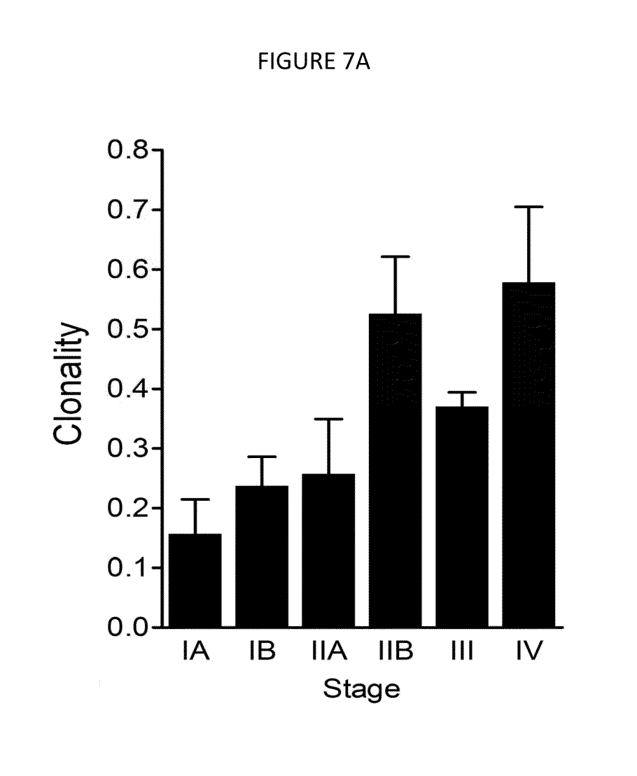

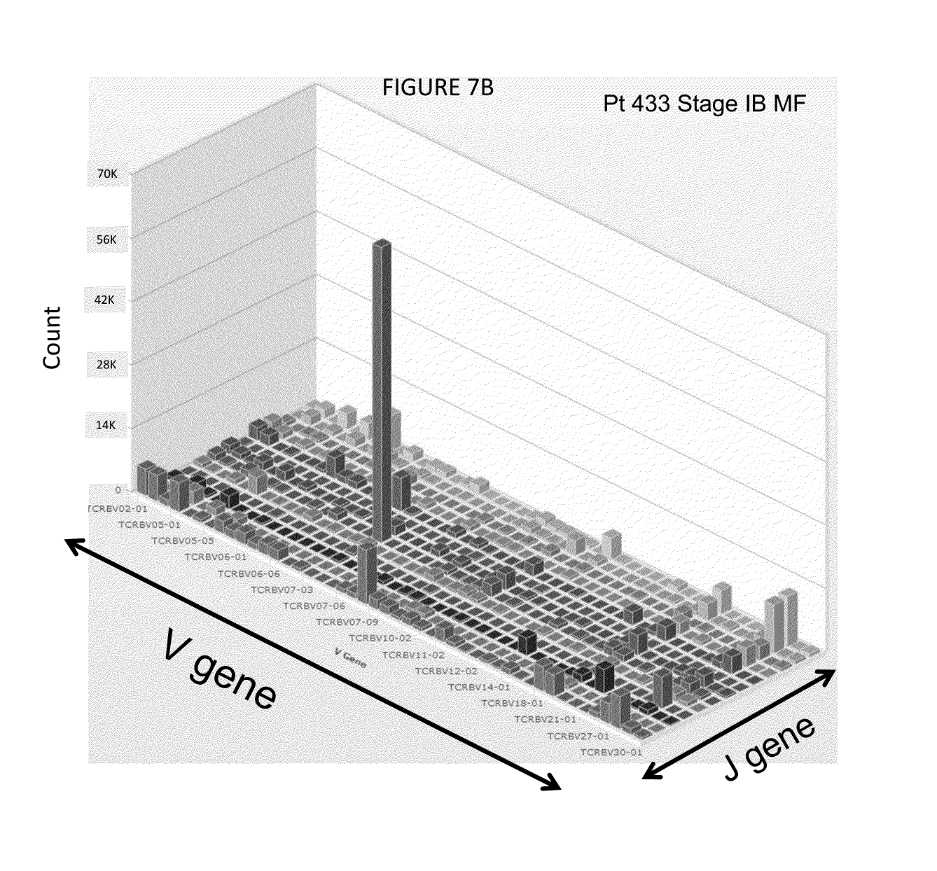

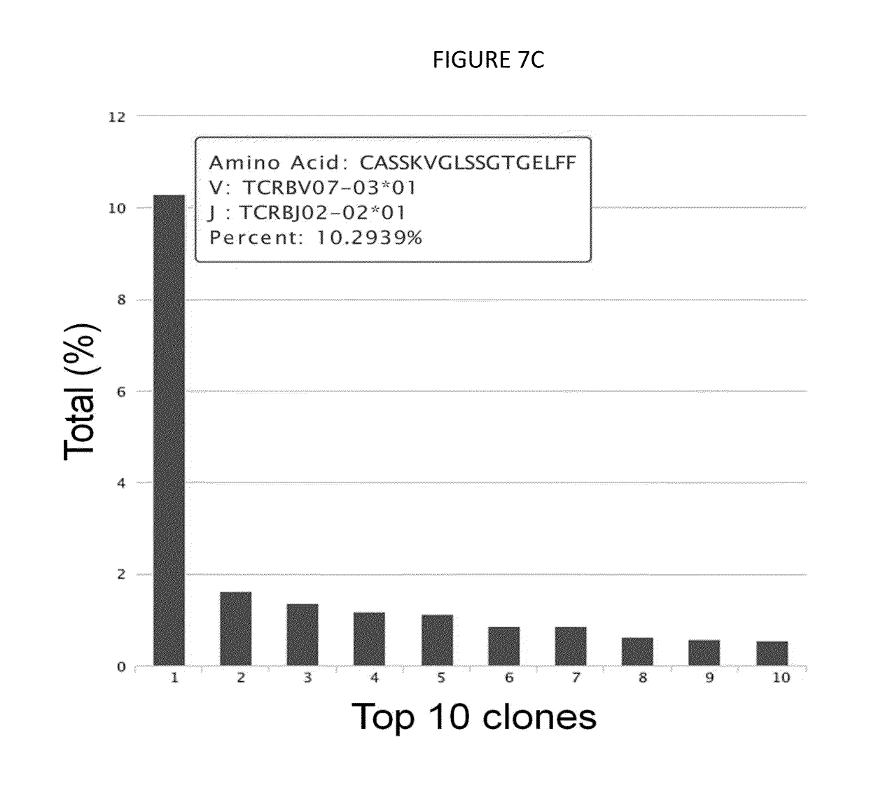

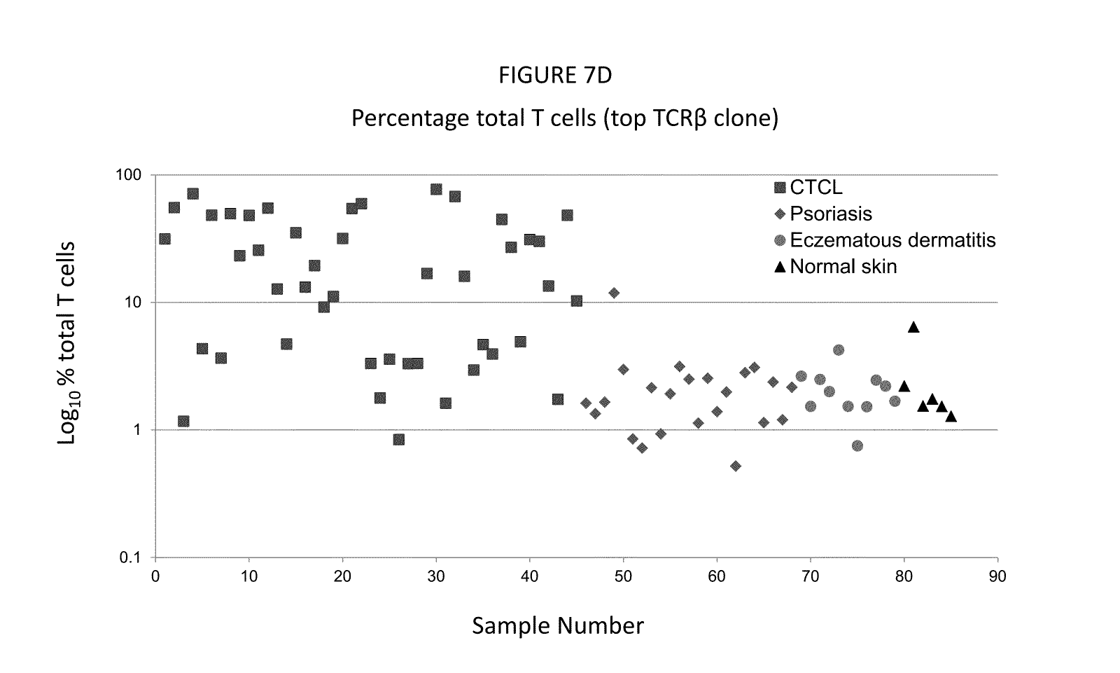

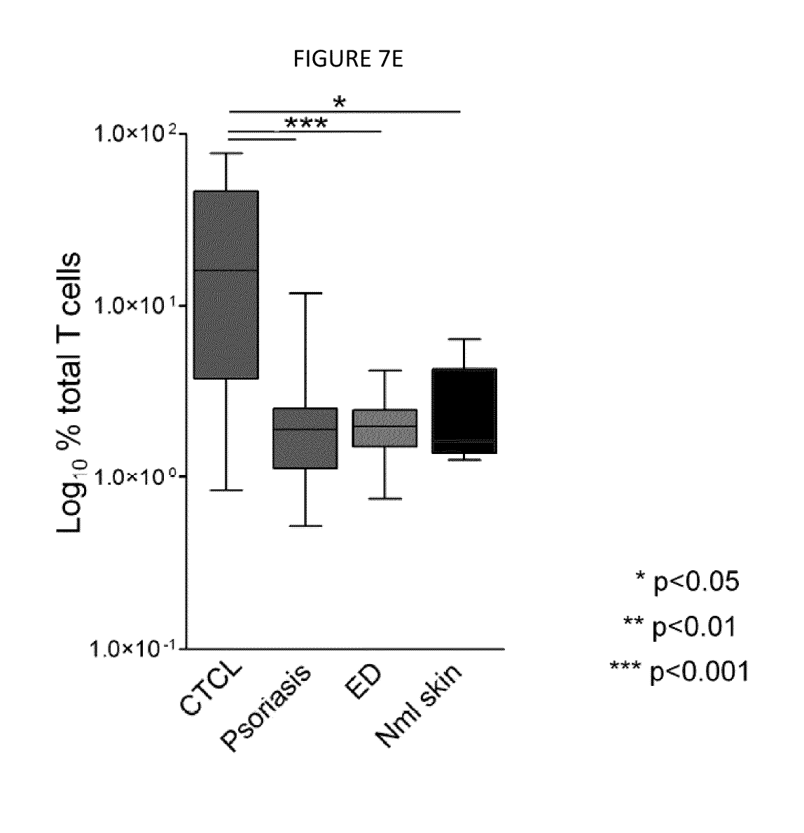

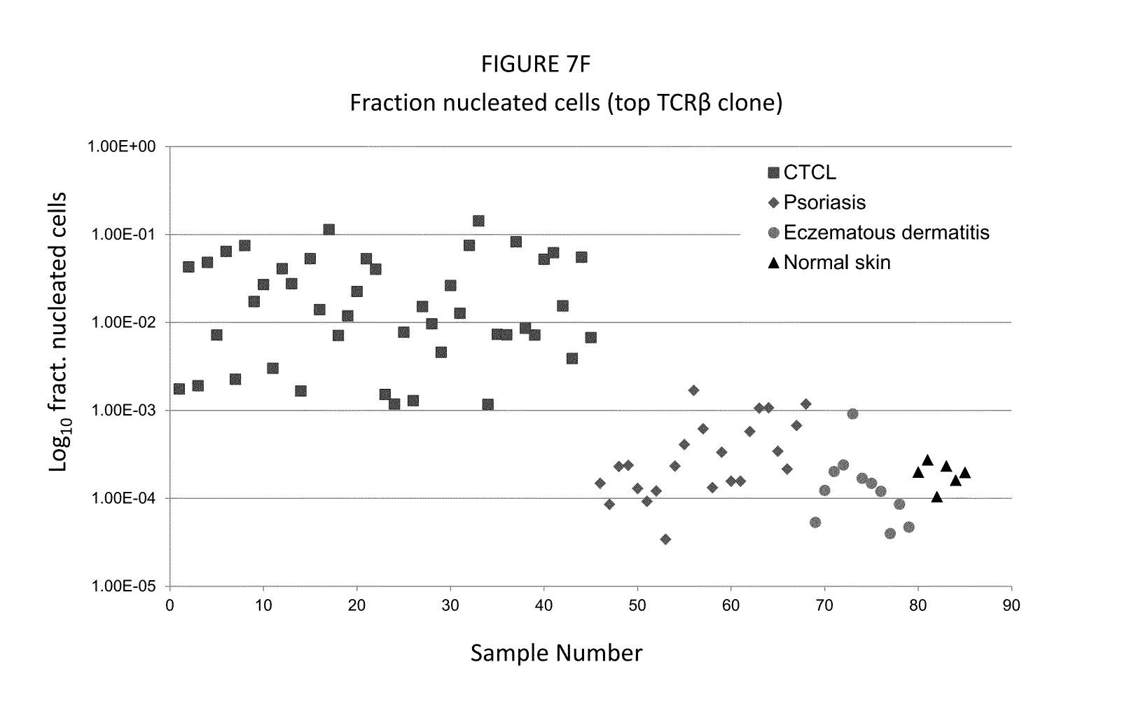

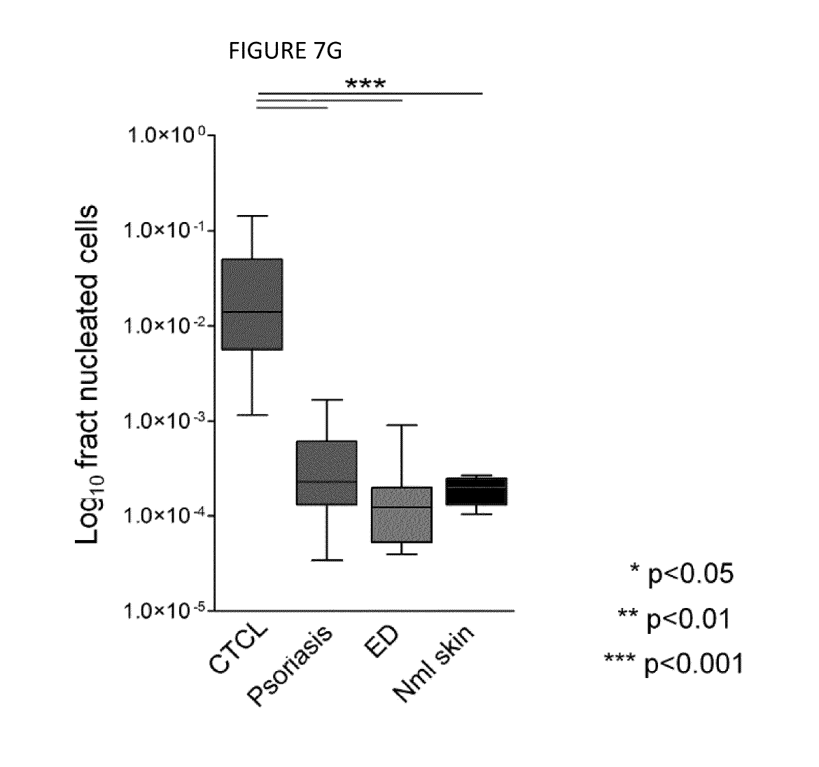

HTS of the TCR Vβ CDR3 regions identified expanded T cell clones in 46/46 lesional samples of CTCL skin (FIGS. 7B-7G). The aggregate V gene and J gene usage for a representative patient with stage IB MF is shown (FIG. 7B), as well as the detailed information regarding the specific amino acid sequence of the single expanded T cell clone in the sample (FIG. 7C). FIG. 7B shows a 3-D histograph of TCR sequencing results that show V gene vs. J gene usages of T cells from a lesional skin sample. Expanded populations of clonal malignant T cells in CTCL skin lesions are shown. The tallest (green) peak includes the clonal malignant T cell population, as well as other benign T cells that share the same V and J gene usage. FIG. 7C shows percent of clones for the top 10 T cell clones in a subject. The individual top T cell clone sequence is shown (1), with detailed information on the CDR3 AA sequence and V and J gene usage. The nine most frequent benign infiltrating T cell sequences are also shown (2-9). In this patient, the malignant T cell clone made up 10.3% of the total T cell population in lesional skin.

However, the top T cell clone expressed as the percentage of total sequenceable T cell genomes in the lesional skin sample was not sufficient to fully discriminate CTCL from other benign inflammatory skin diseases (FIGS. 7D, 17E). FIG. 7D shows the most frequent T cell clone expressed as a percentage of total T cells for individual samples. The most frequent T cell clone expressed as a percentage of total T cells did not completely discriminate CTCL from patients with benign inflammatory skin disease. FIG. 7E shows the most frequent T cell sequences expressed as a percentage of total T cell is shown for aggregate data for subjects with CTCL, psoriasis, eczematous dermatitis (ED) and from the skin of healthy individuals (Nml skin)

The fraction of the total DNA from skin that was contributed by the top cell clone was calculated. This calculation reflected what proportion the top T cell clone made up of the total cells in skin, as opposed to evaluating the expanded clone only as the percentage of total T cells present. When the frequency of the top T cell clone was evaluated as the fraction of total nucleated cells (including keratinocytes, fibroblasts and other cell types), HTS effectively distinguished CTCL from benign inflammatory skin disorders and from healthy skin (FIGS. 1F, 1G). FIG. 7F shows the most frequent T cell clone expressed as the fraction of total nucleated cells for individual samples from subjects with CTCL, psoriasis, eczematous dermatitis (ED) and from the skin of healthy individuals (Nml skin) The most frequent T cell clone expressed as the fraction of total nucleated cells successfully discriminates CTCL from benign inflammatory skin diseases. FIG. 7G shows the most frequent T cell sequence expressed as a fraction of total nucleated cells is shown for aggregate data (G) of samples from subjects with CTCL, psoriasis, eczematous dermatitis (ED) and from the skin of healthy individuals (Nml skin) This analysis allowed discrimination of CTCL from benign inflammatory skin diseases and healthy skin (p <0.001 for all groups). *p<0.05, **p<0.01,***p<0.001.

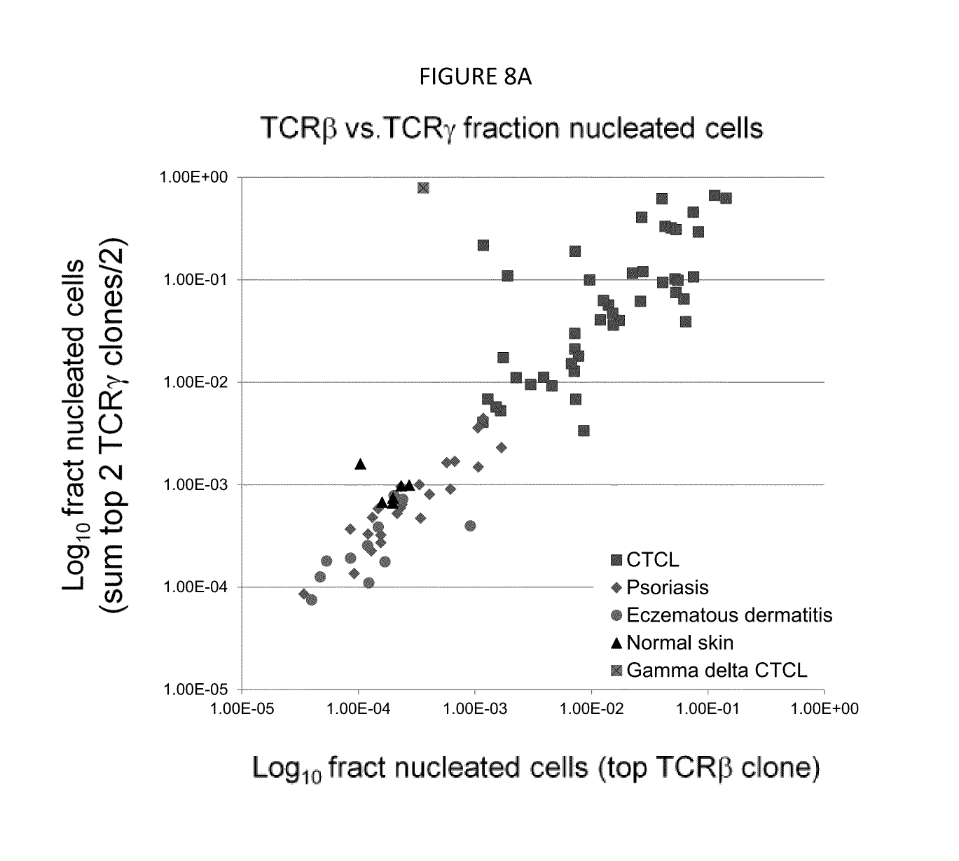

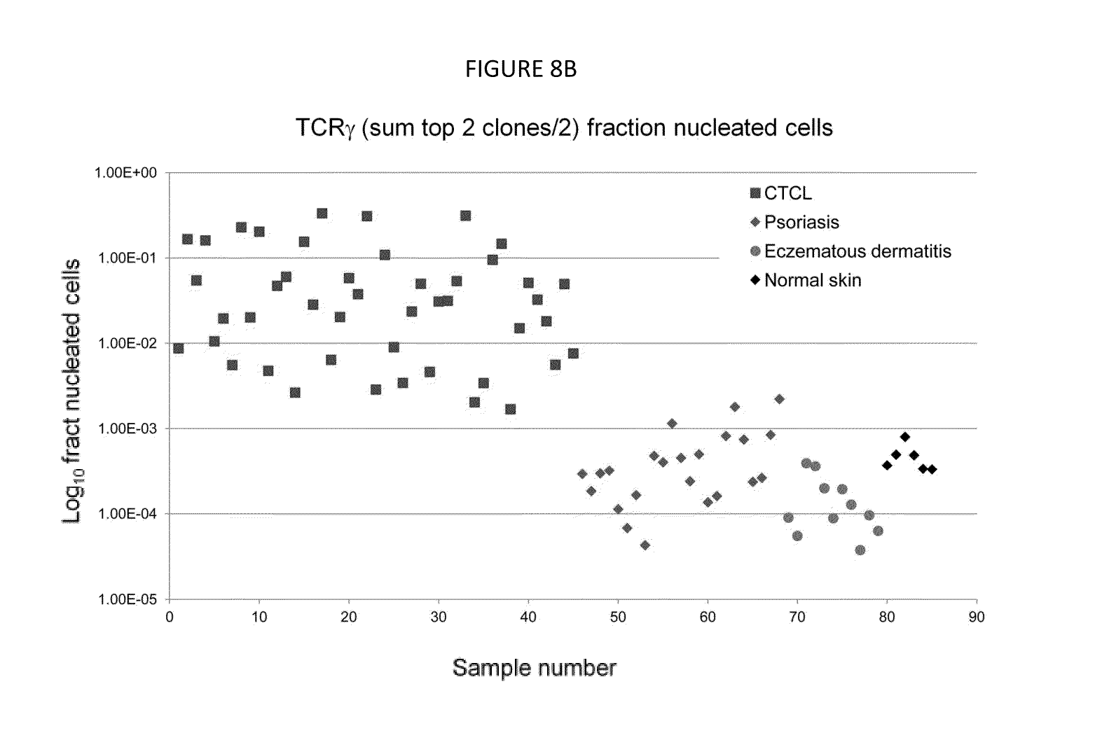

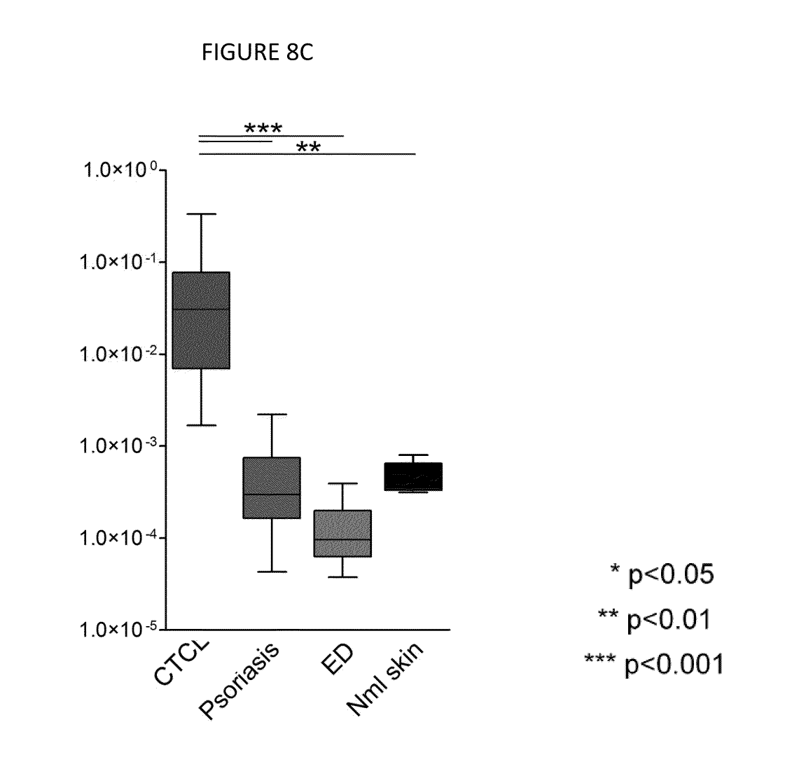

HTS of the TCR Vγ CDR3 genes was also carried out in the same skin samples. Most mature human peripheral blood T cells have two rearranged TCR Vγ genes; the average number of rearranged TCR Vγ genes for an aggregate population of mature human T cells is 1.8 (9). To account for the two rearranged TCR Vγ alleles most often present in a single T cell, the two most frequent top TCR Vγ gene sequences were added in order to calculate the frequency of the top T cell clone in skin samples. Using this approach, there was quite good correlation between TCRβ and TCRγ sequencing results in individual samples. FIG. 8A shows results of high throughput sequencing of both the TCRγ and TCRβ CDR3 regions obtained from skin samples. The top TCRβ sequence and the sum of the top two TCRγ sequences divided by two were expressed as the fraction of total nucleated cells, and the results from TCRγ and TCRβ sequencing are compared. In general, there was close concordance between TCRγ and TCRβ sequencing results. The exception was one patient with a known γδ T cell malignancy, in whom TCRγ sequencing identified a malignant clone but TCRβ did not, consistent with the known lack of TCR Vβ gene rearrangement in γδT cells.

As expected, some samples showed relatively lower clonal frequency by TCRβ analysis (falling to the left of the diagonal line in FIG. 8A); these samples represent T cell clones with two rearranged TCR f3 alleles, which would be expected to make up 17% of the total T cell population. A notable exception was a single case of γδ T cell CTCL, which had a clear clonal T-cell population on TCR Vγ HTS but no detectable clone by TCRβ HTS. In agreement with our results, it has recently been reported that human γδ T cells do not have rearranged TCRβ alleles (9). It would not be expected that this variant of CTCL would be detected by TCRβ HTS. When expressed as the fraction of total nucleated cells in skin, TCR gamma HTS also distinguished CTCL patients from those with benign inflammatory skin disease and from healthy skin (FIGS. 2B and 2C). FIG. 8B shows the sum of the top two TCR γ clones divided by two for individual samples expressed as a fraction of total nucleated cells discriminates CTCL from benign inflammatory skin diseases. FIG. 8C shows the sum of the top two TCR γ clones divided by two for an aggregate of samples expressed as a fraction of total nucleated cells discriminates CTCL from benign inflammatory skin diseases (Psoriasis, ED, Non malignant skin (nml)) (*p<0.05, ***p<0.001).

The method described above can also be used for detecting and diagnosing B cell lymphoid neoplasms and other lymphoid proliferative disorders.

### Example 4

### HTS Diagnosed CTCL in Patients Negative for Clonality by Conventional TCRγ PCR

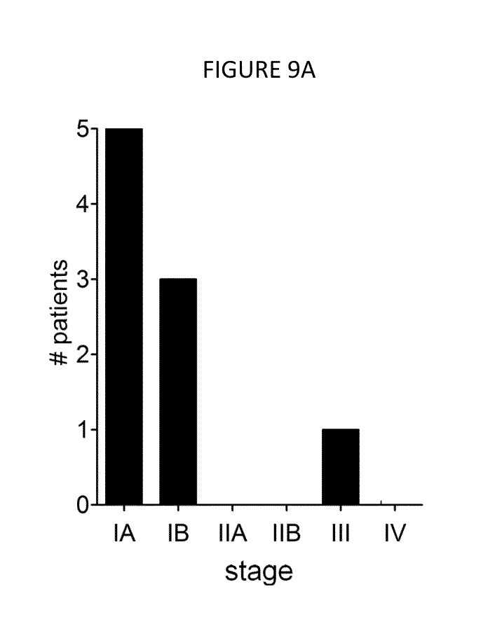

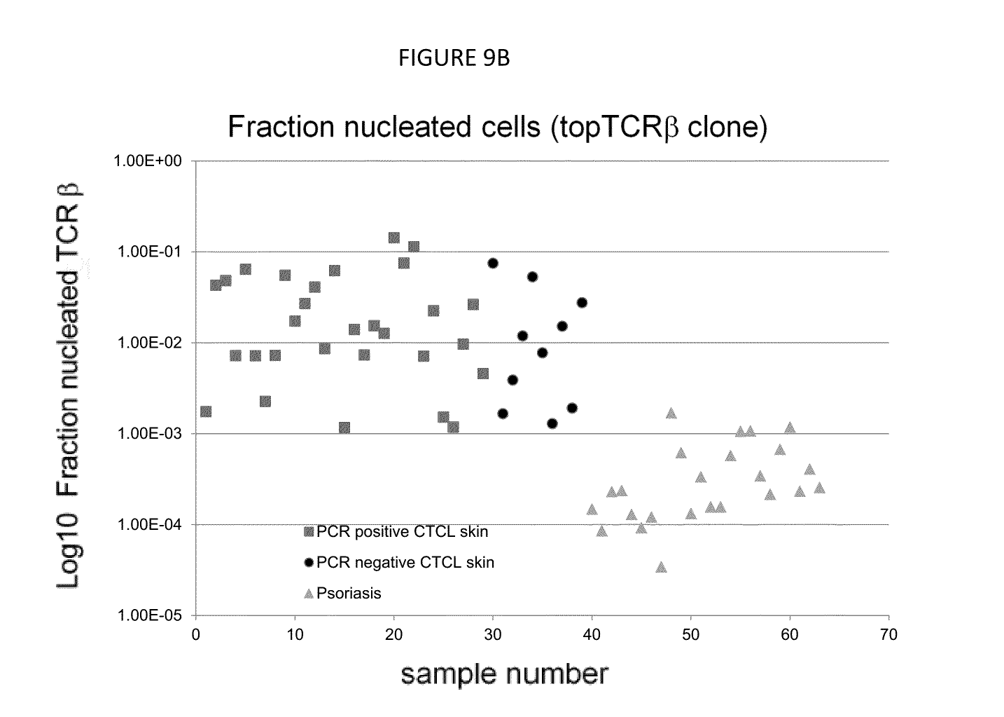

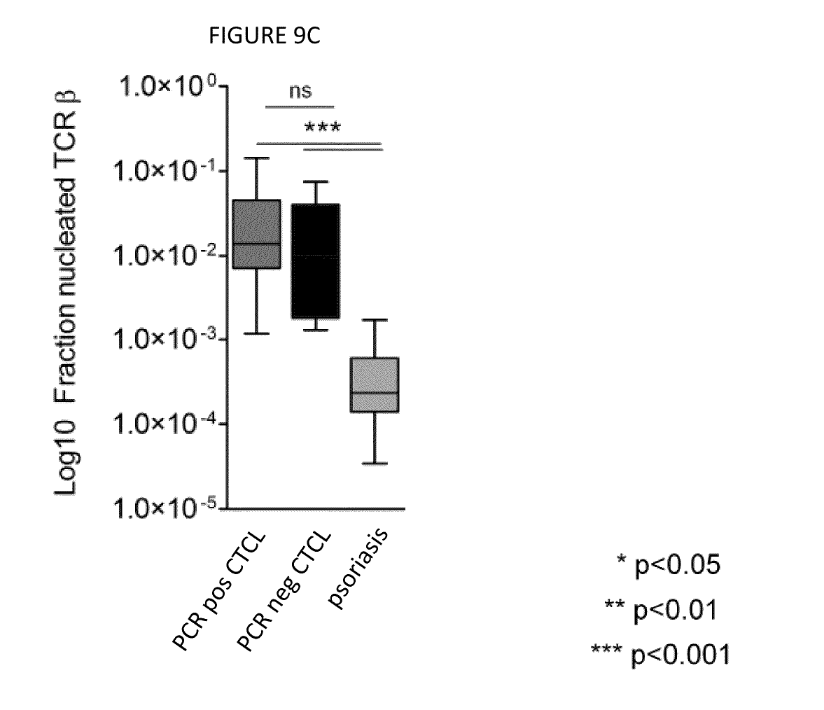

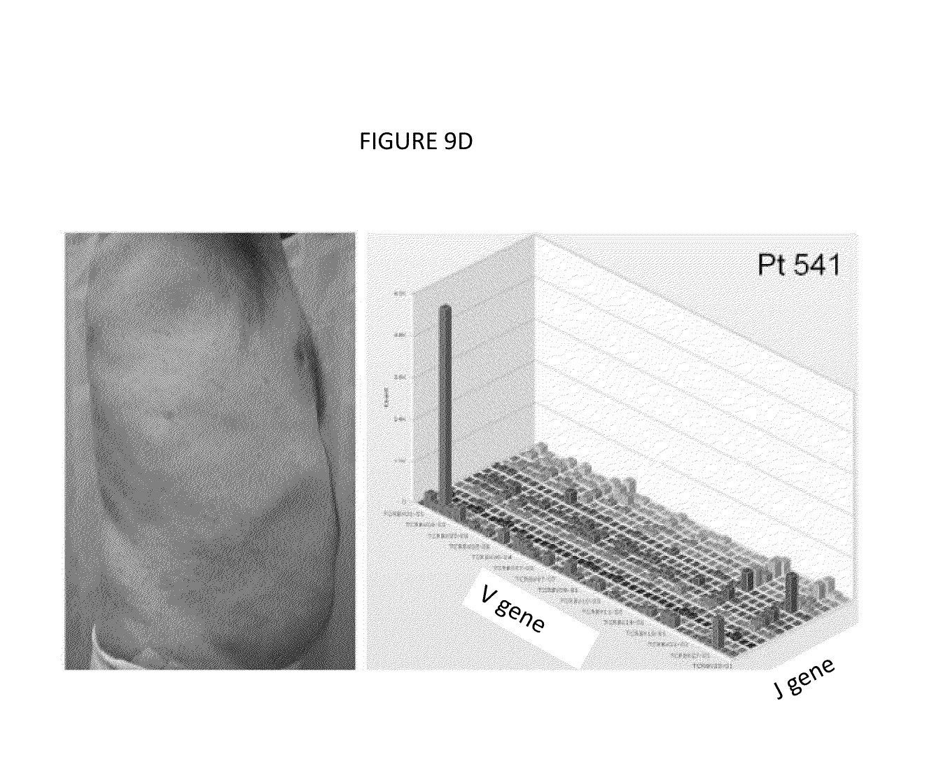

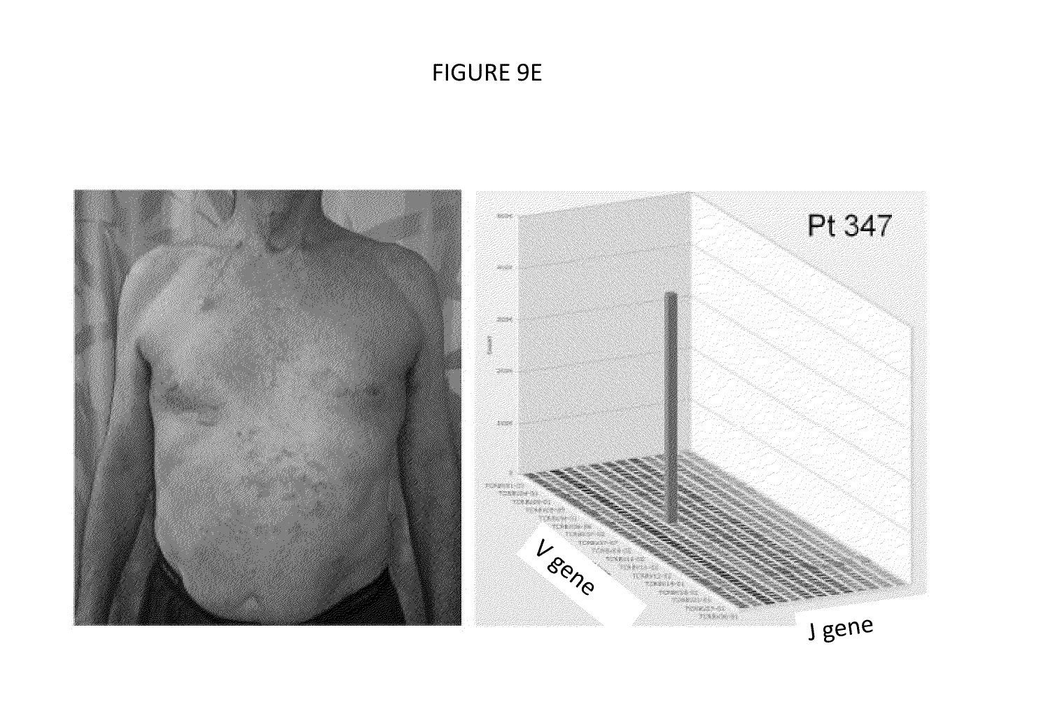

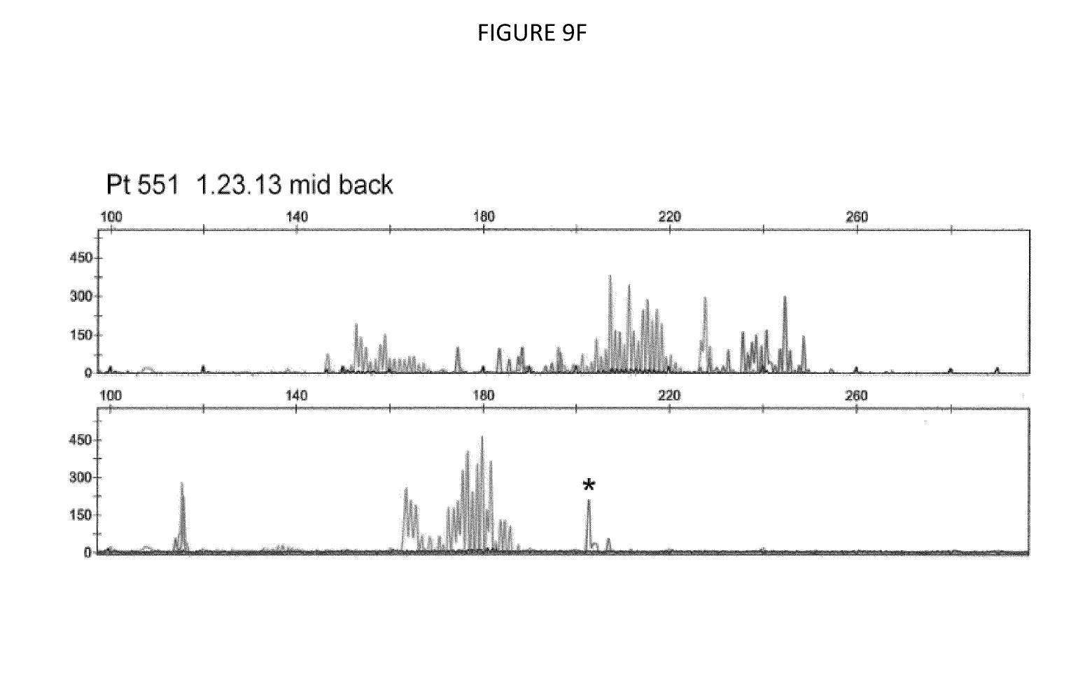

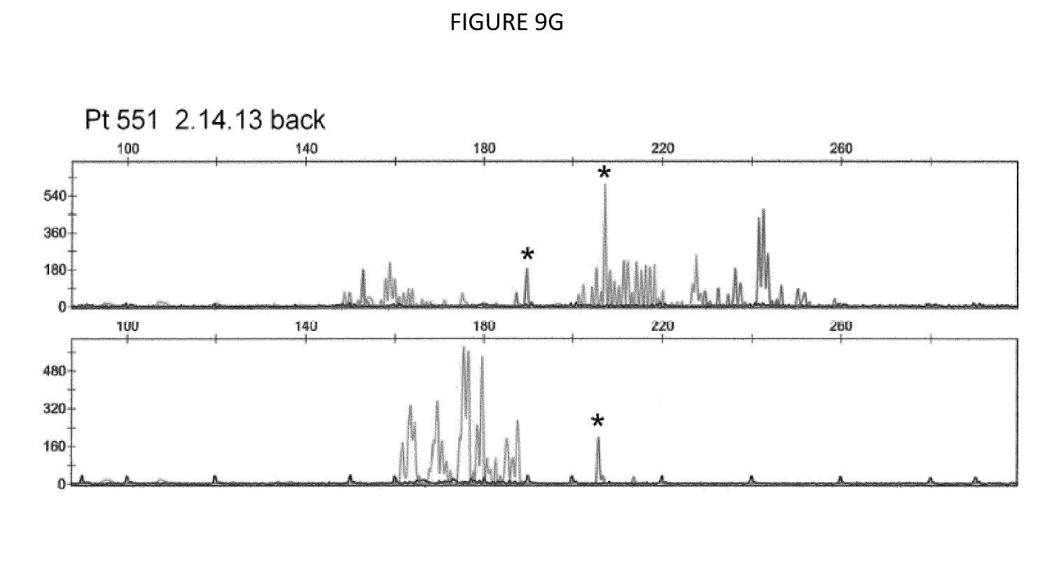

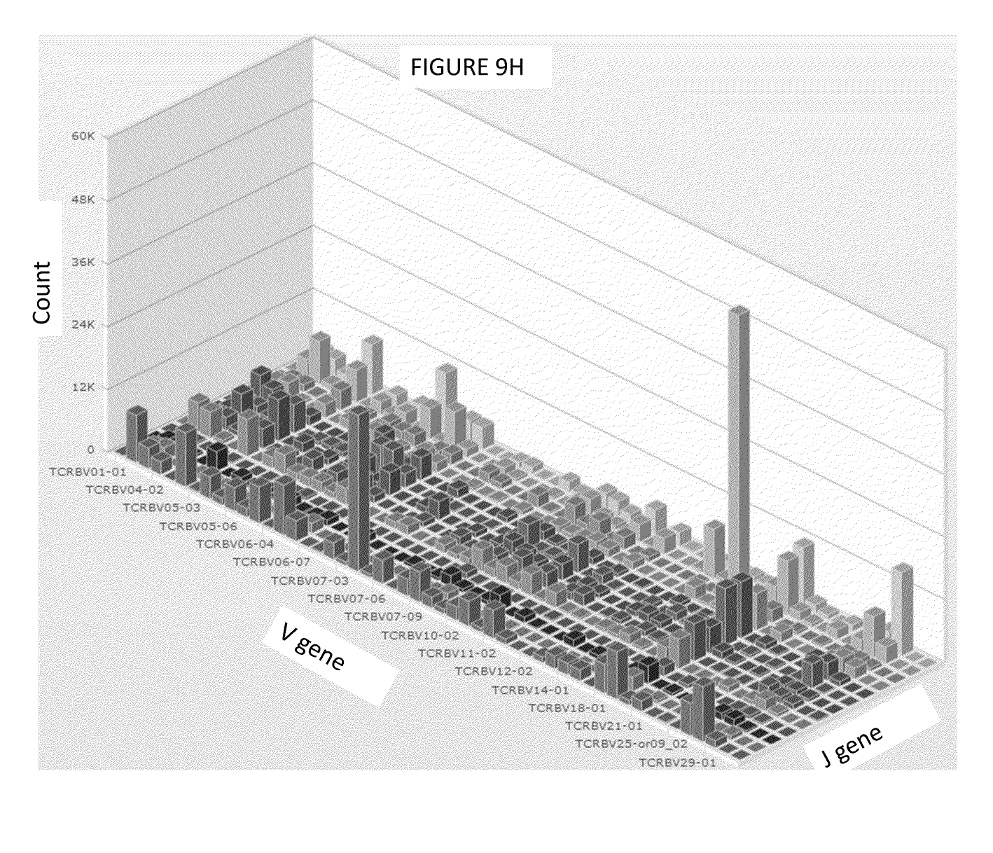

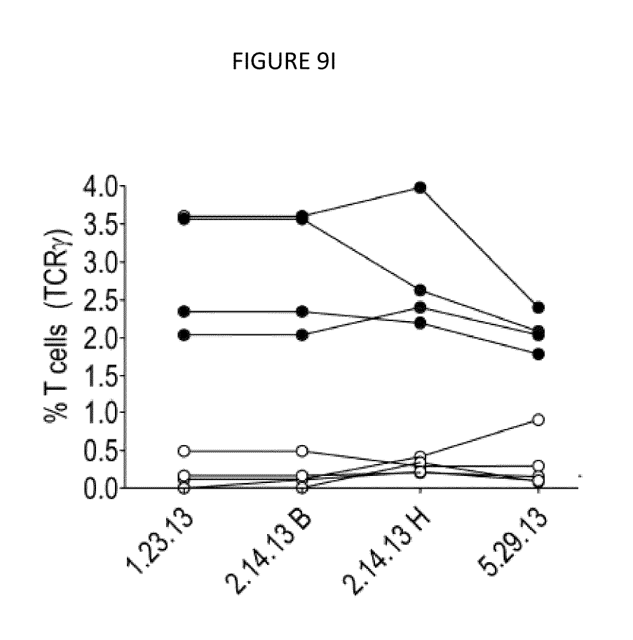

39 patients with clinically confirmed CTCL were evaluated by both HTS and by standard TCRγ PCR and GeneScan capillary electrophoresis (5, 6). HTS identified T cell clones in 39/39 patients compared to TCRγ PCR which identified clonal populations in only 29/39 samples. Nine of the ten patients who had detectable clonality by HTS but not by TCRγ PCR had early stage (IA or IB) disease (FIG. 9A). FIG. 9A shows patients diagnosed for CTCL using high throughput sequencing (HTS) in various stages, who were found negative for clonality by conventional TCRγ PCR. HTS and TCRγ PCR were carried out on skin biopsies from 39 CTCL patients; HTS identified clones in 39/39 CTCL patients compared to TCRγ PCR, which identified clonal populations in 29/39 samples. The stages of the nine of the ten CTCL patients with clones detected by HTS but negative for clonality by TCRγ PCR are shown.

However, the prevalence of the expanded T cell clone as assayed by HTS was similar in PCR negative and PCR positive patients (FIGS. 8B and 8C), suggesting that TCRγ PCR was negative in these patients for reasons other than a very low predominance of the malignant clone. The clinical photographs and HTS results in two representative patients with stage IB CTCL are shown (FIGS. 9D and 9E). FIG. 9D shows a clinical photo and HTS results for Patient 541, who had pathology proven stage IB CTCL, but in whom TCRγ PCR did not detect a clonal population. FIG. 9E shows a clinical photo and HTS results for patient 347. TCRγ PCR was negative and pathology was equivocal, but HTS results of TCRB demonstrated a clear malignant clone.

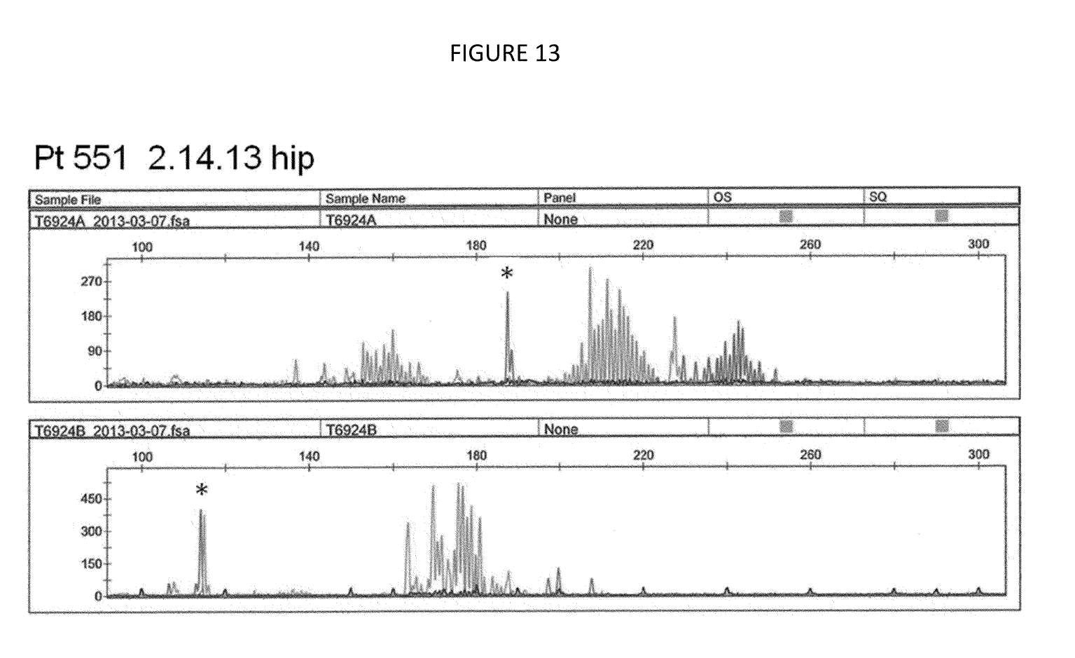

HTS demonstrated clear expanded T cell clones, but TCRγ PCR was read as negative for clonality in both cases. HTS was particularly helpful in patient 551, a patient with stage IA CTCL in whom three separate biopsies were all read as negative for clonality by TCRγ PCR (FIGS. 9F, 9G, and 13). FIG. 9F shows TCR γ PCR results for two biopsies from patient 551. In total, four skin biopsies were sent for HTS, and three biopsy samples were studied by TCRγ PCR. All samples were negative for clonality by TCRγ PCR, but 4/4 were positive for clonality by HTS. FIG. 9G shows TCRγ PCR results for two biopsies of patient 551. The results for the third sample are shown in FIG. 13. Asterisks indicate peaks noted by the pathologist within the expected areas, but none were judged significant enough to designate as a clonal population. FIG. 13 shows TCR γ chain PCR results for a third biopsy from patient 551. Asterisks indicate peaks noted by the pathologist within the expected areas, but none were judged significant enough to designate as a clonal population.

TCR β HTS demonstrated the presence of two high-frequency TCR β sequences (FIG. 9H), suggesting the presence of either a single T cell clone with two rearranged β alleles or two dominant T cell clones. FIG. 9H shows that HTS of TCRVβ demonstrated the presence of two distinct Vβ clonal sequences, denoting the presence of either a single malignant clone with two rearranged TCRβ alleles or two separate malignant T cell clones.

TCR γ HTS demonstrated the presence of four predominant TCR gamma sequences in all four biopsies from this patient, demonstrating two expanded T cell clones were present in this patient, each with two rearranged TCR gamma alleles. FIG. 9I shows results of HTS of TCRγ, which demonstrated the presence of four dominant gamma chain clones in all four biopsies (black circles), confirming that there were two clonal malignant T cell populations in this patient, each with one rearranged TCRβ allele and two rearranged TCRγ alleles. The five most frequent benign T cell TCRγ sequences are also shown for comparison (white circles). ***p<0.001.

The method described above can also be used for detecting and diagnosing B cell lymphoid neoplasms and other lymphoid proliferative disorders.

### Example 5

### HTS Discriminated CTCL Recurrences from Benign Inflammation, Provided Accurate Assessment of Responses to Therapy and Facilitated Early Diagnosis of Disease Recurrence in Both the Skin and Blood of Patients with CTCL

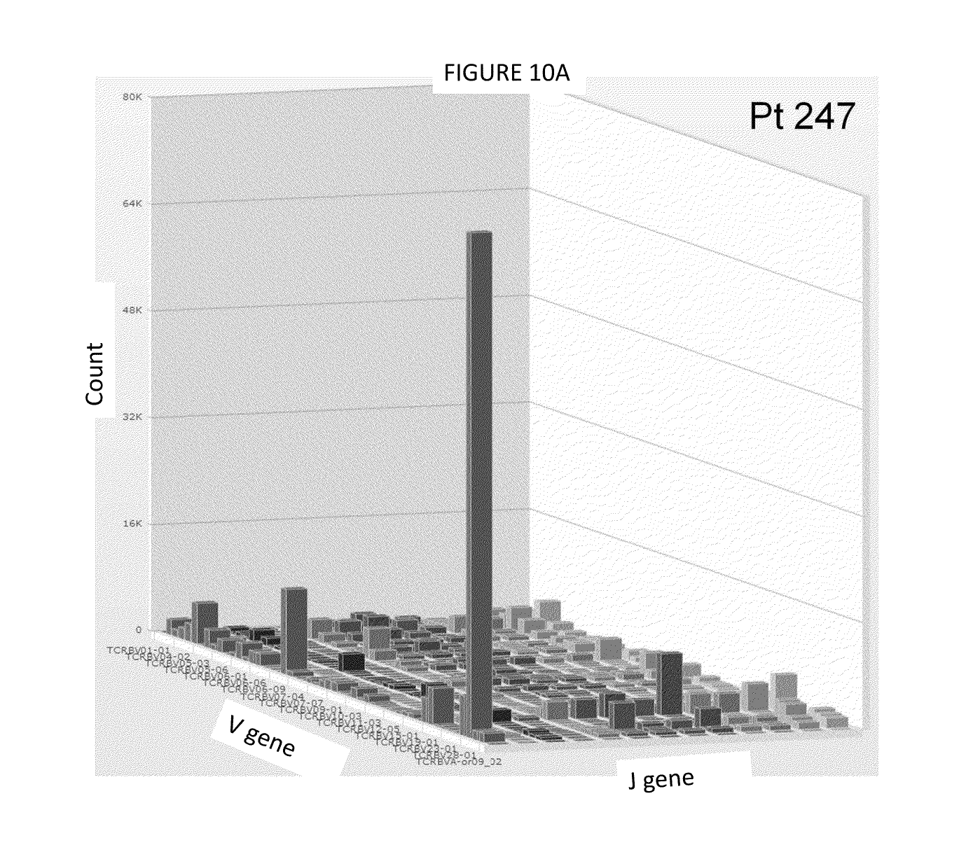

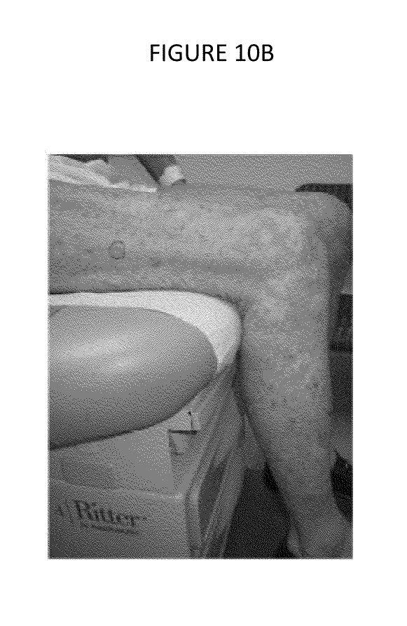

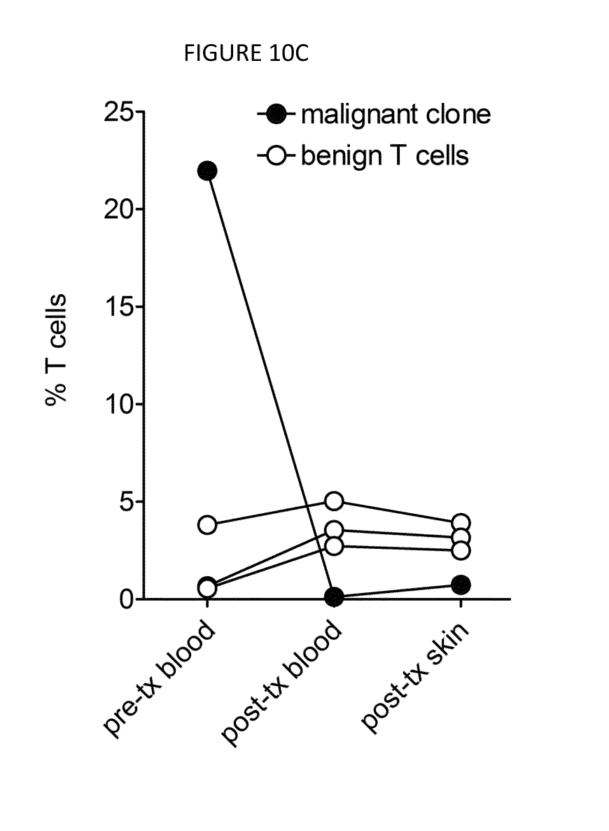

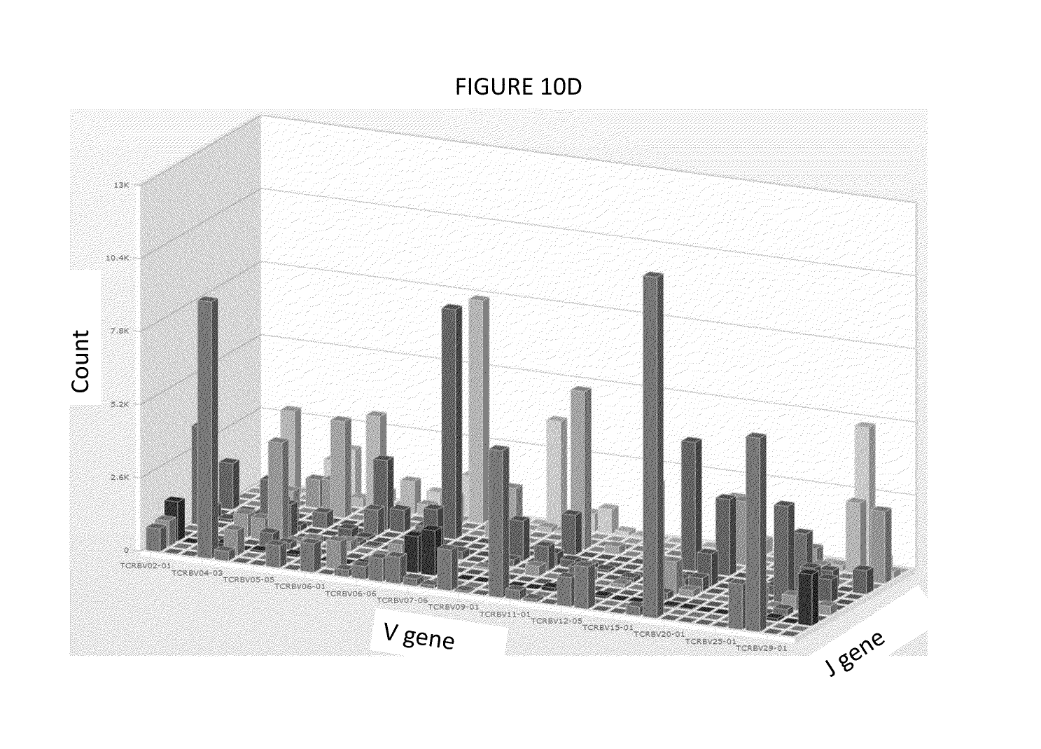

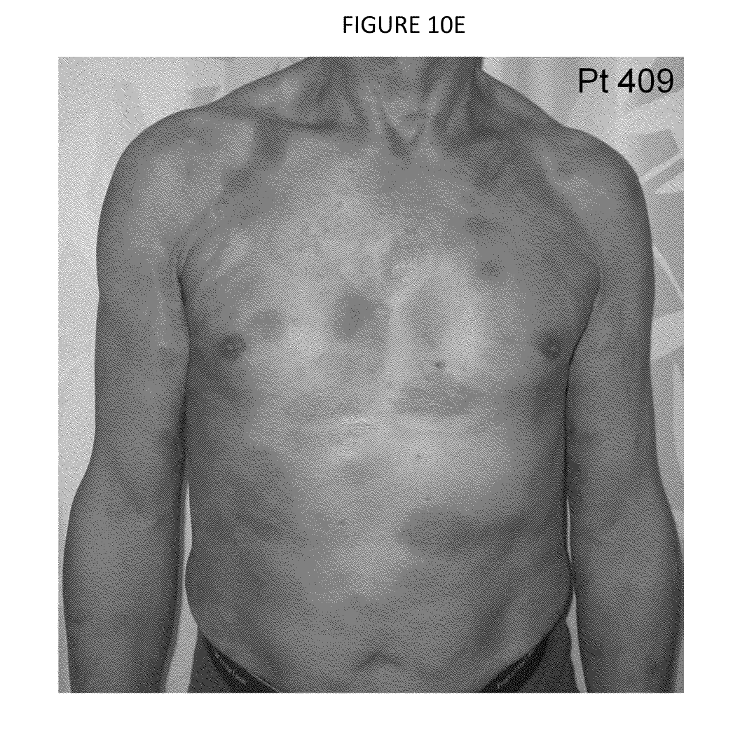

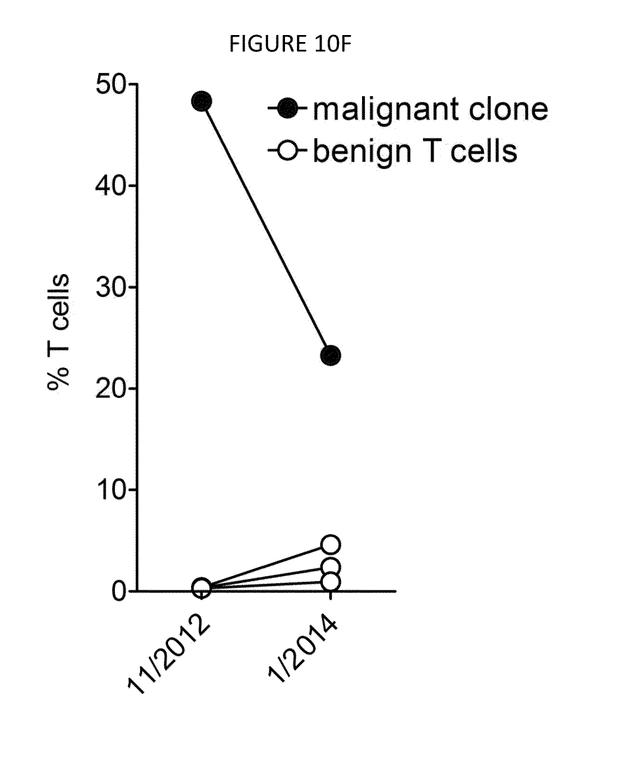

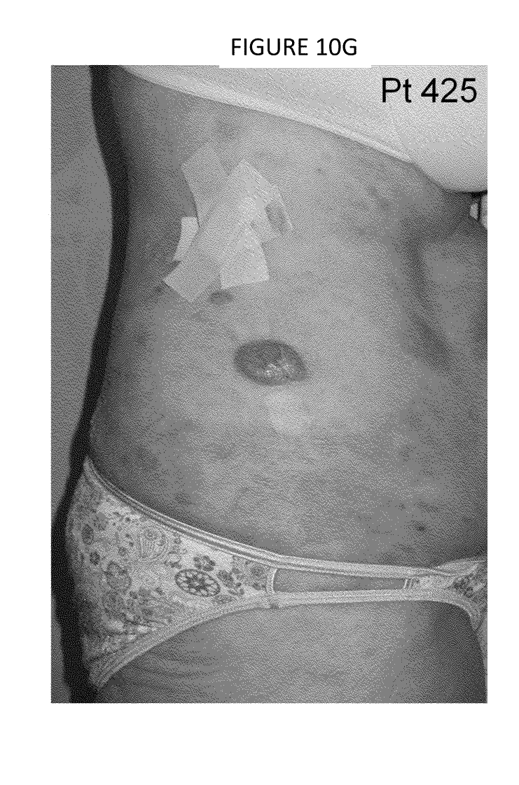

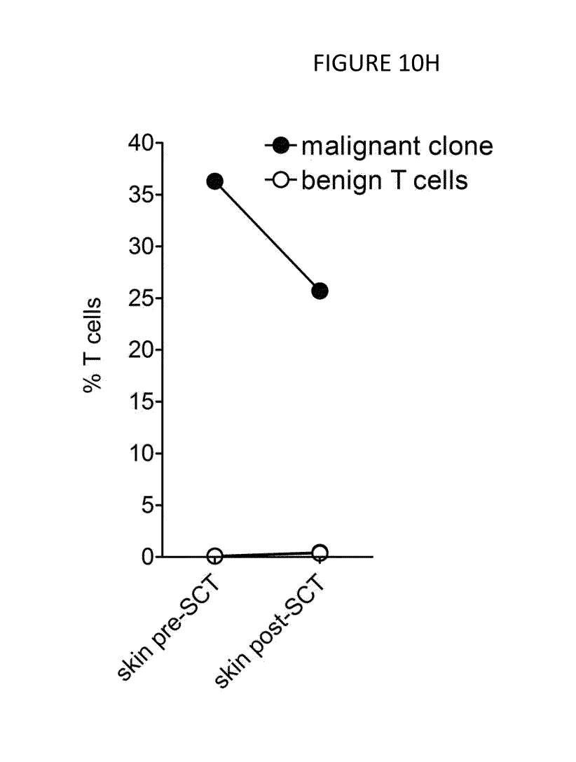

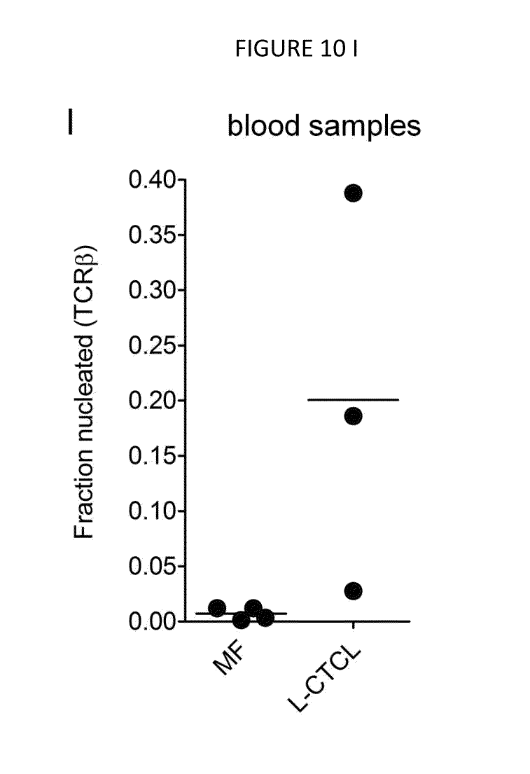

By virtue of its ability to identify and quantify clonal T cells, HTS greatly facilitates the care of patients with CTCL. HTS was remarkably effective in distinguishing early CTCL recurrences from benign inflammation of the skin, a challenge that occurs when patients develop a cutaneous eruption following what otherwise appears to be successful therapy. Patient 247 had stage IB CTCL with peripheral blood involvement that was detectable by both HTS (FIG. 10A) and by clinical flow cytometry analysis. The patient was begun on low dose alemtuzumab along with valacyclovir and sulfamethoxazole/trimethoprim prophylaxis. He experienced a rapid improvement in his skin erythema and pruritus but after his ninth cycle of alemtuzumab, he developed an inflammatory cutaneous eruption that was worrisome for recurrence of CTCL (FIG. 10B). FIG. 10A illustrates that HTS discriminates CTCL recurrences from benign inflammation, provides accurate assessment of responses to therapy and facilitates early diagnosis of disease recurrence in both the skin and blood of patients with CTCL. FIG. 10A shows that prior to therapy, TCRVβ HTS demonstrated a clear malignant clone in blood. FIG. 10B shows a photograph of patient 247. Histopathology was suggestive of a drug hypersensitivity response but a T cell dyscrasia could not be ruled out. HTS clearly demonstrated loss of the malignant T cell clone from both blood and skin (FIG. 10C), supporting the diagnosis of a nonmalignant hypersensitivity response. FIG. 10C shows a graph of HTS results of blood and lesional skin, which showed clearance of the malignant T cell clone from blood and skin, confirming this was a benign inflammatory dermatitis. Sulfamethoxazole/trimethoprim was discontinued, and the patient recovered completely with topical steroids and narrow band UVB therapy. HTS demonstrated the presence of a diverse population of skin resident memory T cells remaining in this patient's skin after alemtuzumab therapy (FIG. 10D). FIG. 10D shows a 3D histogram of diverse populations of T cells remaining within the skin of alemtuzumab treated patients. TCRVβ HTS of the skin of patient 247 while on alemtuzumab is shown. This patient had no circulating T or B cells but a diverse population of T cells remained in skin

HTS was also useful for its ability to track individual malignant T cell clones in the blood and skin over months and years in a particular patient. Patient 409 had stage IIA CTCL and was first studied by HTS in 2012, after treatment with electron beam and brachytherapy and again in 2014, after initiation of gemcitabine. HTS demonstrated an identical clonal T-cell population in the skin at both time points, reduced but still frequent after gemcitabine therapy (FIGS. 10E, 10F). FIG. 10E shows a photograph of skin of patient 247. FIG. 10F shows a graph of HTS results of the malignant clone and benign T cells (as % T cells). HTS demonstrated an identical clonal T-cell population in the skin at both time points, reduced but still frequent after gemcitabine therapy. The malignant clone (black circle) and three most frequent benign T cell clones (white circles) are shown.

In addition, HTS rapidly and efficiently diagnosed early disease recurrences. Pt 425 had a history of recalcitrant stage IIB CTCL with CD30⁺ large cell transformation. HTS performed prior to SCT identified the malignant T cell clone. She underwent a matched donor stem cell transplant and appeared well until 10 months after transplantation, when she developed a new right abdominal lesion. HTS demonstrated recurrence of the same malignant T cell clone demonstrable before SCT. Prompt withdrawal of systemic immunosuppression and skin directed narrowband UVB therapy induced a complete remission.

Lastly, HTS was effective in identifying and quantifying expanded T cell clones in blood samples from patients with CTCL. HTS did not identify expanded T cell clones in the blood of patients with MF, but did detect expanded clonal T cells in patients with clinical evidence of peripheral blood disease (leukemic CTCL, L-CTCL), as evidenced by positive clinical flow cytometry analyses (FIGS. 10A-I).

### Example 6

### For Patients with Skin Limited Disease and No Clinical Involvement of Peripheral Blood, HTS Demonstrated Hematogenous Spread of Small Numbers of Malignant T Cells

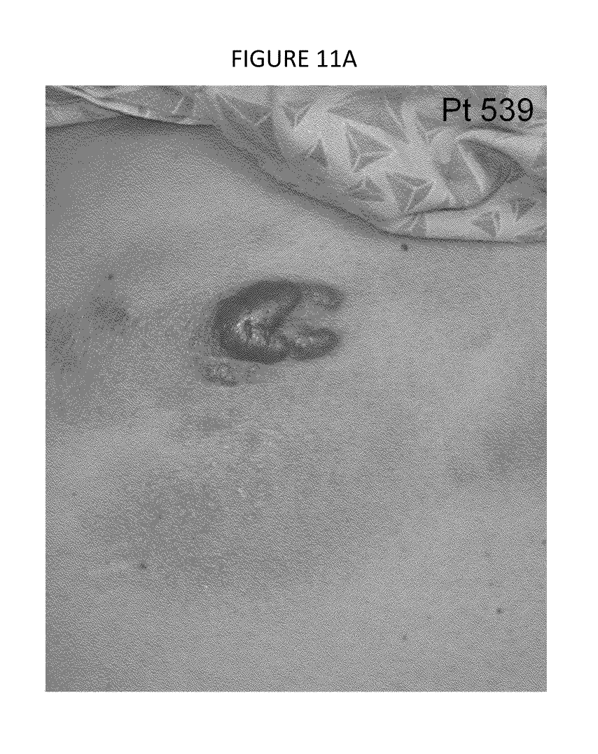

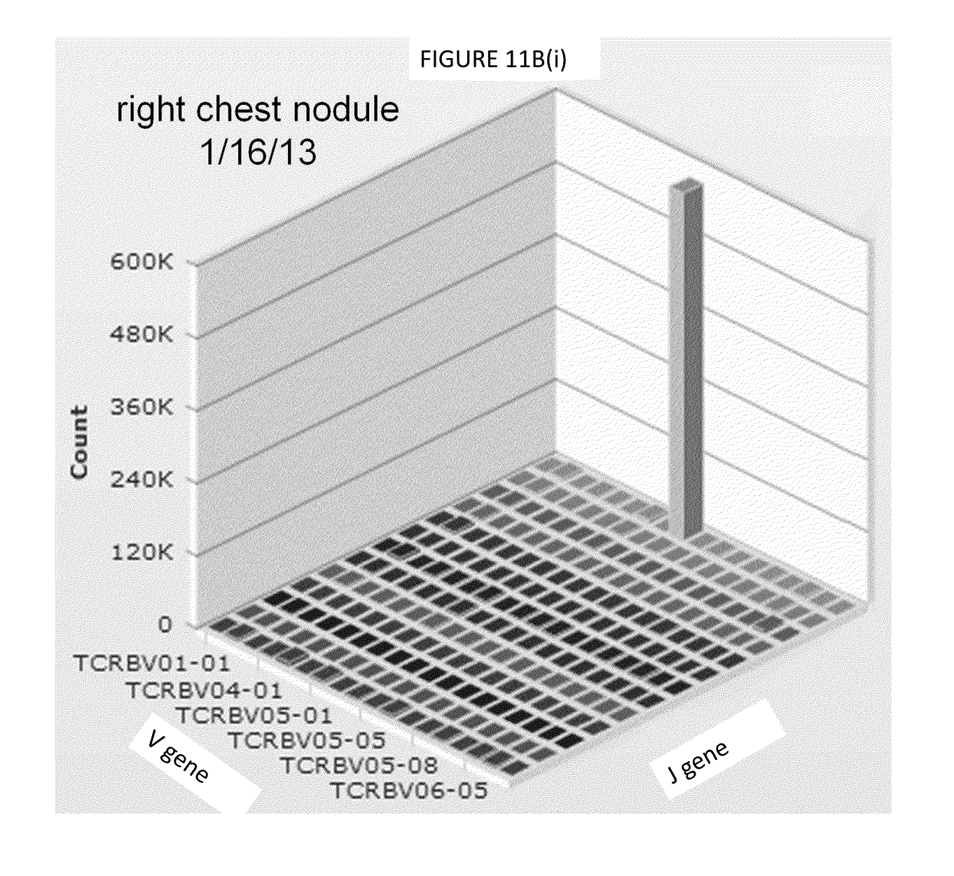

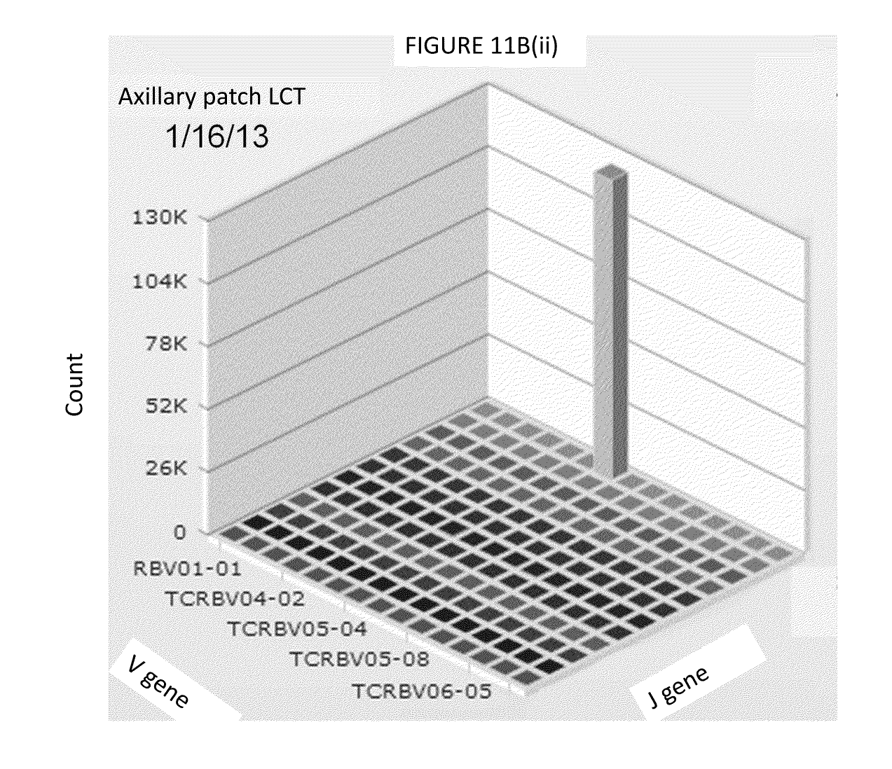

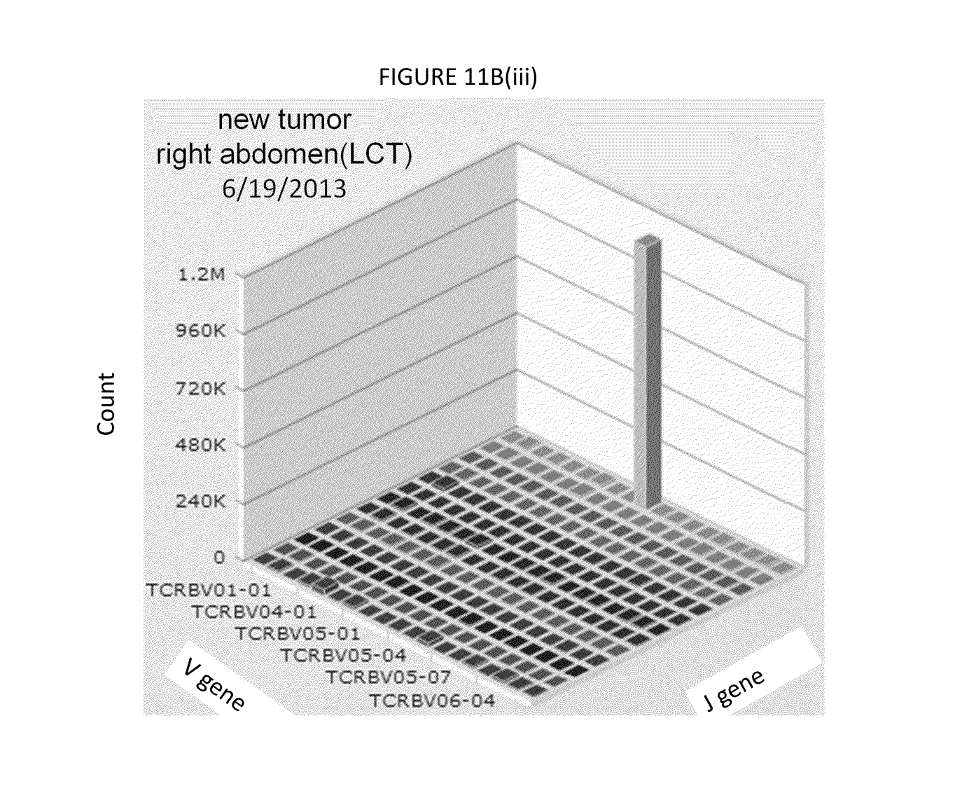

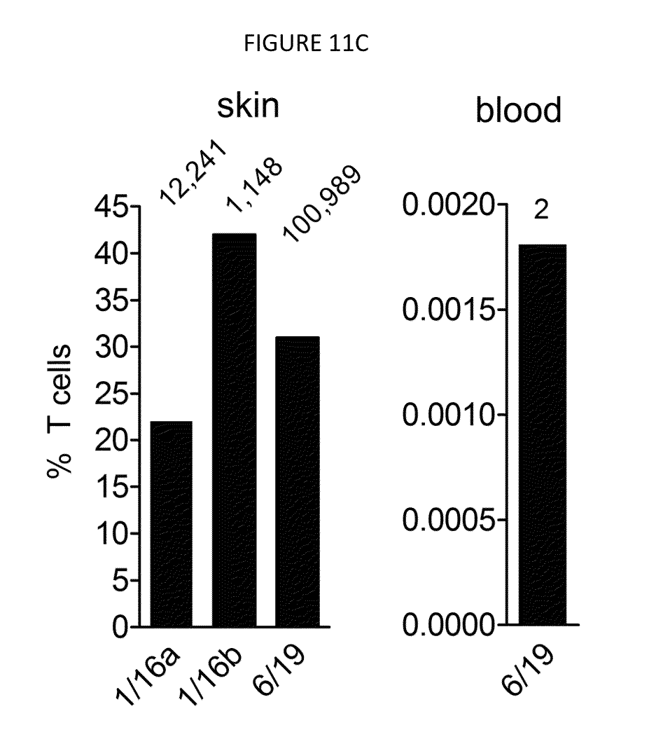

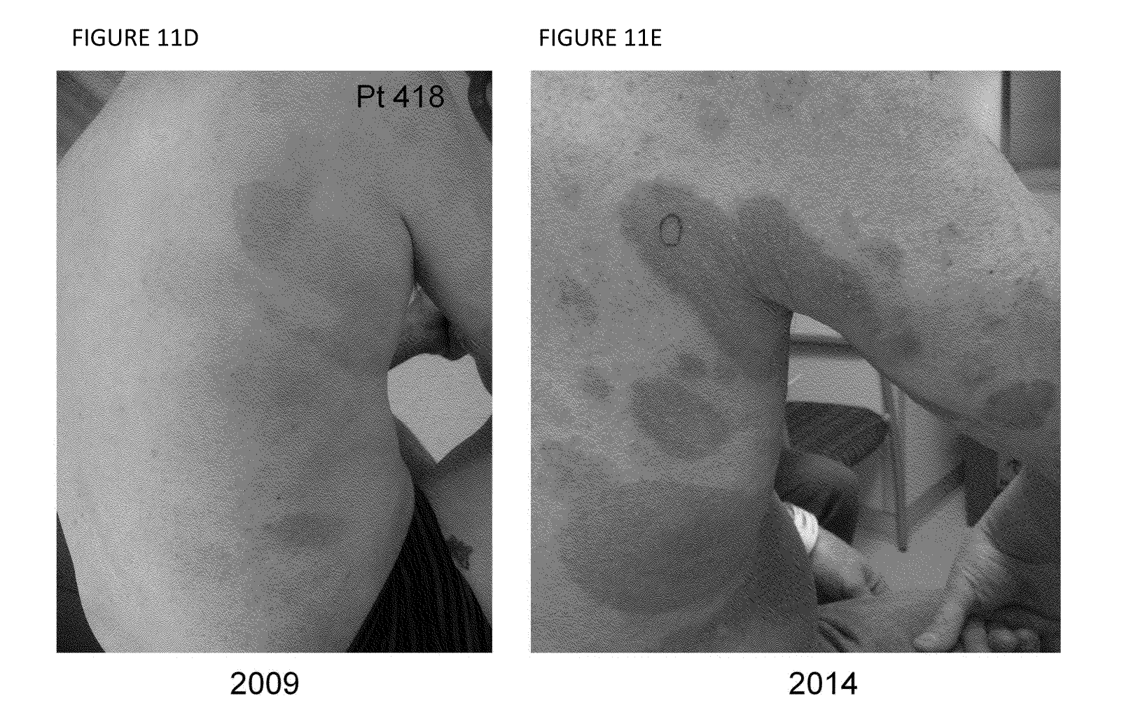

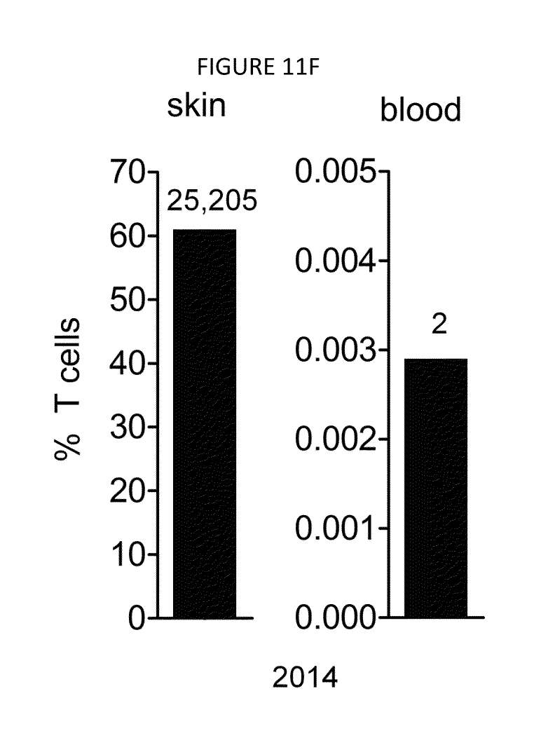

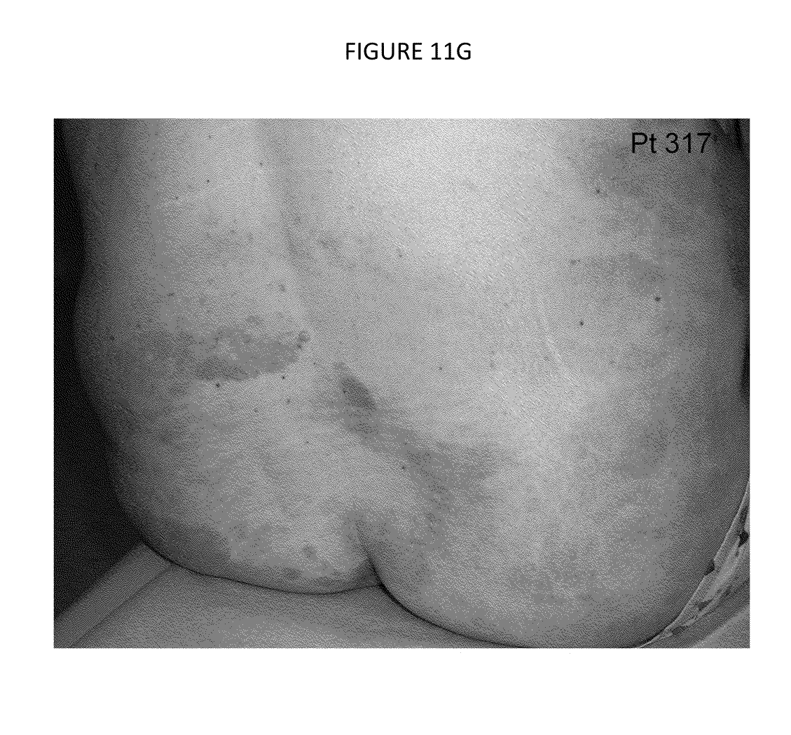

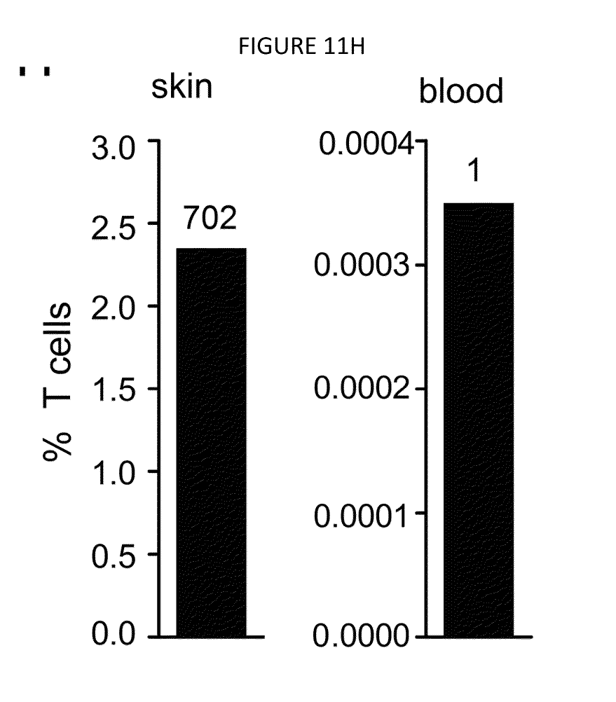

The methods of the invention not only enhance clinical care but also provide novel biologic insights into CTCL with possible implications for disease staging. For example, patients with skin limited CTCL and no evidence of peripheral blood involvement by clinically available flow cytometry analyses can develop new CTCL skin lesions distant from previously involved areas. Patient 539 had stage II CTCL with patchy (LCT on histopathology) and nodular skin (MF on histopathology) skin lesions (FIG. 11A) and no evidence of peripheral blood disease by clinical flow analyses. The skin lesions improved after local radiation therapy but the patient developed a new tumor at a previously uninvolved distant site five months later. HTS studies demonstrated the same malignant T cell clone in all three skin lesions. Evaluation of the peripheral blood at the time of the development of the new skin lesions also demonstrated low but detectable numbers of the same malignant clone within the peripheral circulation. At both time points, clinical flow studies were negative for peripheral blood involvement. Patient 418 had a long-standing Stage IIB folliculotropic CTCL previously controlled with a number of therapies including narrowband UVB, psoralen and UVA light (PUVA), oral bexarotene, Denileukin diftitox, electron beam radiation, pralatrexate and nitrogen mustard (FIG. 11D). In 2014, he was seen with thickening of existing lesions and development of new skin lesions (FIG. 11E). Clinical flow analyses were negative for peripheral blood involvement. HTS analysis of blood and skin samples demonstrated the presence of an abundant T cell clone in skin that was demonstrable in low levels in peripheral blood (FIG. 11F). Patient 317 had an over 40 year history of long-standing large plaque parapsoriasis since childhood and was eventually diagnosed with mycosis fungoides in 2007 (FIG. 11G). In 2014, he presented with a 1.5 year history of worsening disease with new areas of involvement. HTS analyses of skin and blood demonstrated the presence of a clonal T-cell population in skin that was also present in very low numbers within the peripheral blood. In summary, in 3/3 patients with skin limited disease, there was no evidence of peripheral blood disease by flow cytometry analyses. However, HTS demonstrated the hematogenous spread of low numbers of clonal T cells.

### Example 7

### HTS is a Valuable Research Tool that Provides Novel Insights into the Biology of CTCL

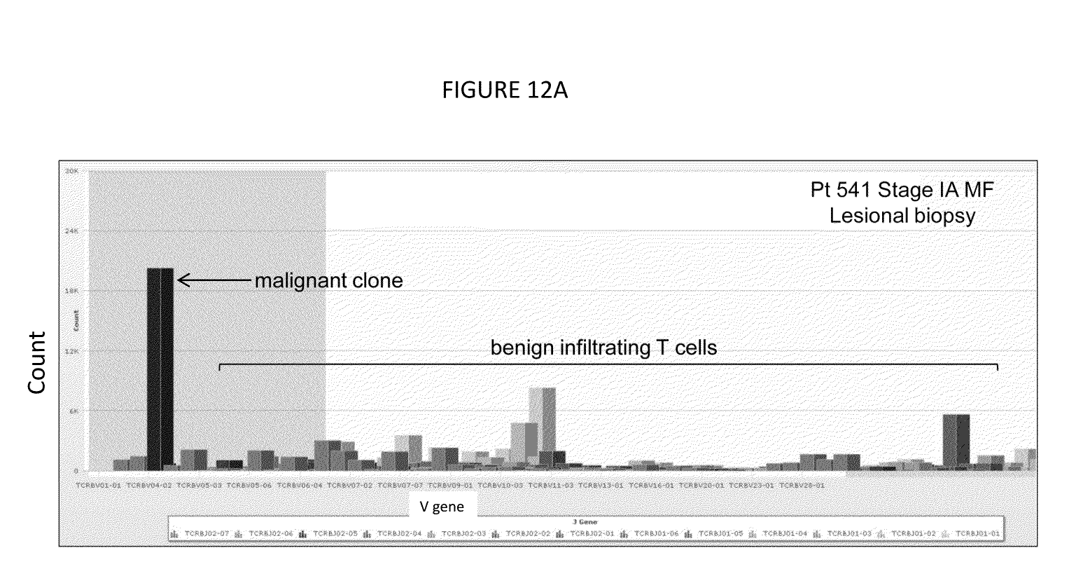

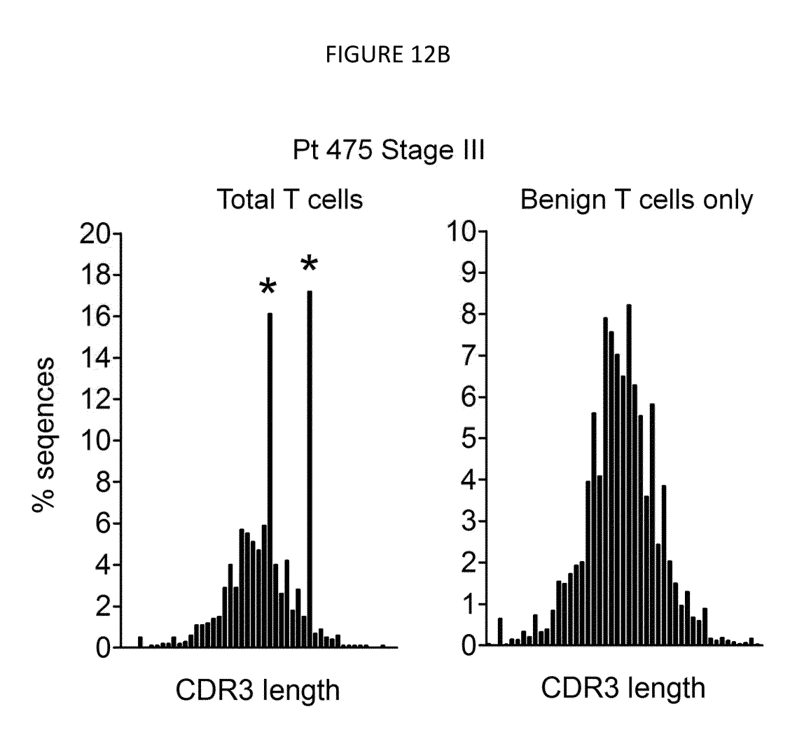

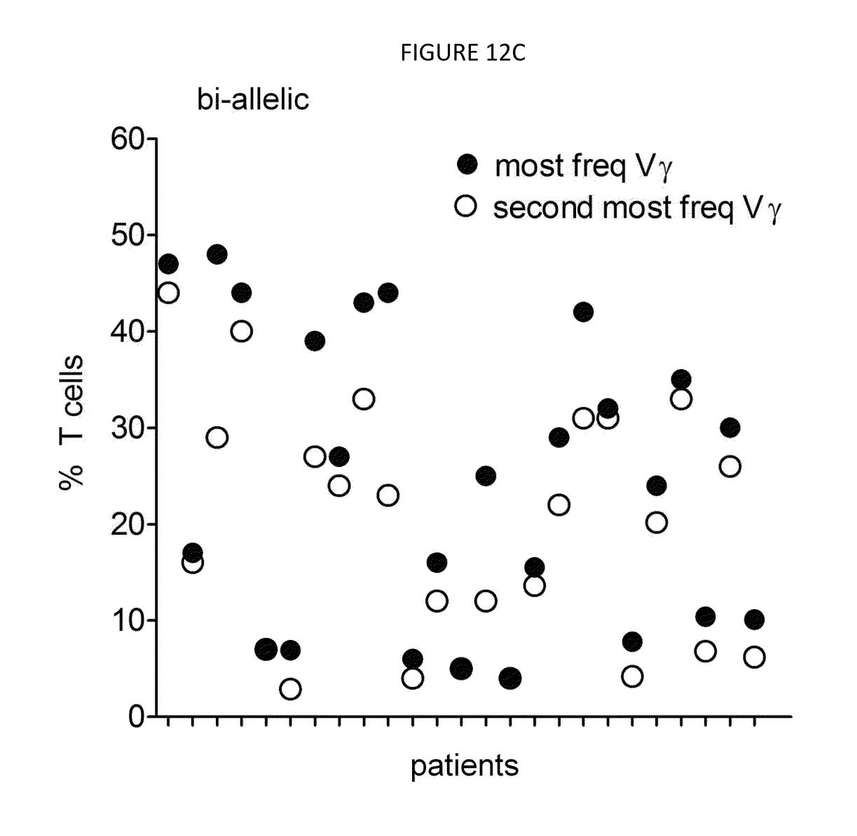

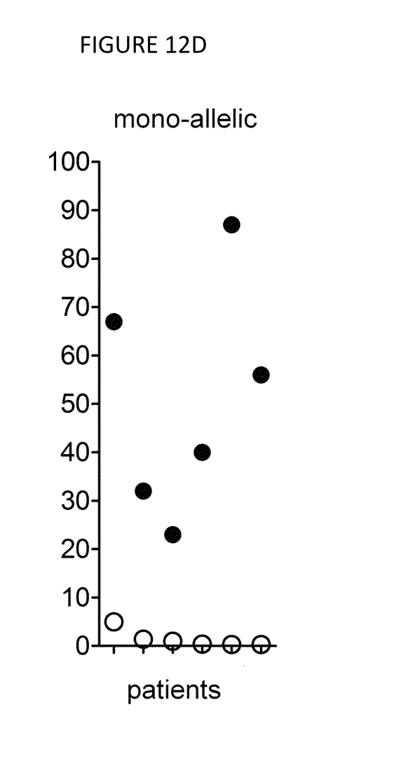

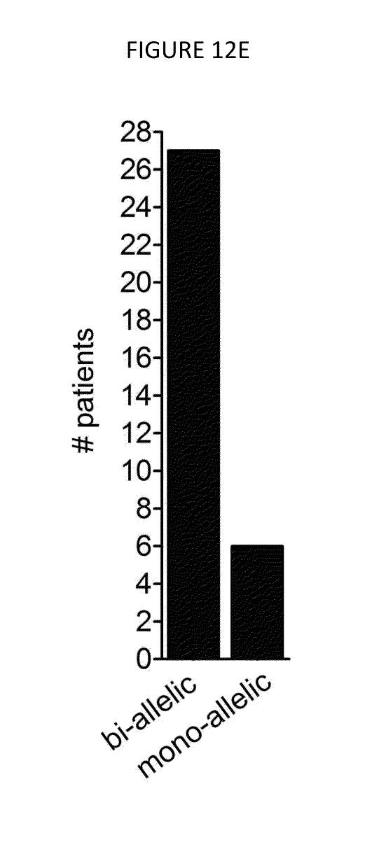

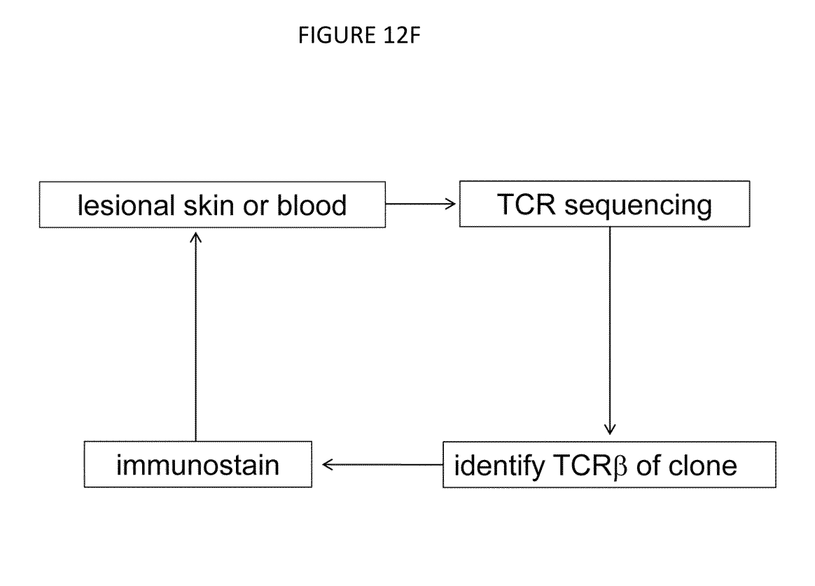

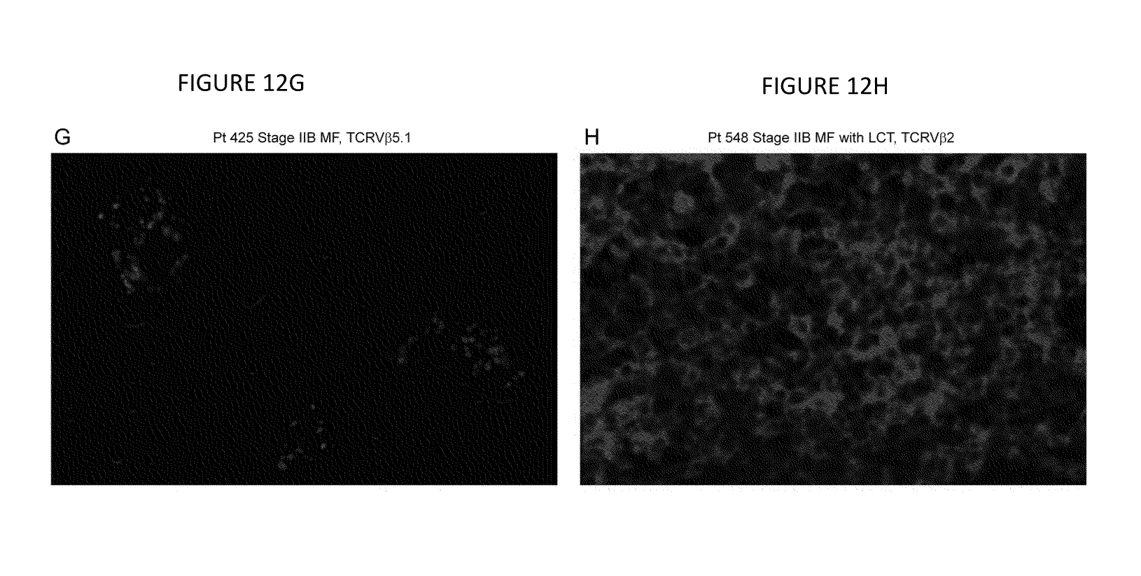

The methods of the invention are a powerful technology that provide the exact sequences and numbers of every T cell a particular biologic specimen. In CTCL, HTS allows the study of both the malignant clonal T cells and benign infiltrating T cells within the skin lesion (FIG. 7A). The wealth of information generated by HTS can be used to selectively examine the numbers and diversity of benign infiltrating T cells in CTCL skin lesions, something not possible with previous methods. For example, TCRγ HTS can be used to generate graphs similar to classic spectra typing which depict the CDR3 lengths of total T cells (including the two rearranged TCRγ alleles of the malignant clone, FIG. 12B left panel) or of the benign T cells in isolation (FIG. 12B right panel). This ability to isolate malignant from benign infiltrating T cells and study the diversity and numbers of each provides an unparalleled opportunity to evaluate both malignant T cell and responding benign T cells within CTCL skin lesions.

TCRγ HTS also allows determination of how many rearranged TCRγ alleles exist in a given malignant clone and this provided a novel insight into the underlying biology of CTCL. CTCL patients in whom TCRγ HTS demonstrates the presence of two similarly abundant TCRγ sequences are patients with T-cell clones that have both TCRγ alleles rearranged (bi-allelic, FIG. 12C). Likewise, patients with a single most frequent TCRγ sequence by HTS are those who have T cell clones with a single rearrangement of the TCRγ allele (mono-allelic, FIG. 12D). Having two independent and distinct TCRγ marker sequences can provide additional verification of the malignant clone. In our specimens, 27 CTCL T cell clones were clearly bi-allelic and six were clearly mono-allelic (FIG. 12E). On average, each malignant T cell clone had 1.8 rearranged TCRγ alleles, in complete agreement with the values observed in human mature T cells (9). The observation demonstrates that CTCL is a malignancy arising in mature T cells, not a malignancy of immature T cells or lymphoid progenitor cells.

Lastly, TCRβ HTS provides an unparalleled opportunity to identify the TCR Vβ subunit utilized by the malignant T cell clone. Approximately 60 to 70% of TCRVβ subfamilies can be recognized by commercially available monoclonal antibodies, allowing for immunostaining of the malignant T cells in samples of lesional skin or in blood. T cells expressing the TCR Vβ subunit utilized by the malignant T cell clone were immunostained in cryosections from lesional skin of two MF patients (FIGS. 12G, 12H). Immuno-identification of the malignant T cell clone could be used to selectively study malignant T cell gene expression, cytokine production and other features utilizing the techniques of immunostaining, laser capture micro-dissection followed by RT-PCR or transcriptional profiling.

High throughput sequencing of the TCR CDR3 regions, the hyper variable portions of the TCR γ and β genes that make up the antigen recognition domains, provides an exact fingerprint for every T cell in a particular biologic specimen. The exact unique nucleotide sequences that make up each individual T cell clone are identified, the number of these cells relative to other T cells in the sample is measured and the total number and overall diversity of T cells is provided (8). This single, one step study provides an unprecedented amount of information about T cells in a biologic specimen.

The methods of the invention detected an expanded T cell clone in the skin lesions and blood of all CTCL patients studied. The skin lesions of 39 of our patients were studied by both HTS and TCRγ PCR. HTS identified expanded T cell clones in all of these patients whereas TCR V gamma PCR positive in only 74%. HTS was particularly effective in detecting expanded T cell clones in patients with earlier stages of disease. Surprisingly, patients who had clones identified by HTS but not by TCRγ PCR were not necessarily those with lower numbers of clonal T cells in skin lesions, suggesting that factors other than a lower sensitivity of TCR gamma PCR may interfere for the detection of clonal T cell populations by this technique. However, taken together, our results suggest that HTS is superior to TCR gamma PCR in detecting clonal T cell populations in patients with CTCL.

One of the clinical challenges in diagnosing patients with early-stage disease is discriminating CTCL from benign inflammatory skin disorders such as psoriasis and atopic dermatitis, which these lymphomas can clinically resemble. To be a successful diagnostic test, HTS must therefore not only detect expanded T cell clones in patients with CTCL but must also be able to successfully discriminate CTCL from benign inflammatory skin disorders. Clonal T cell expansion occurs in healthy immune responses and clonal expansion of individual, antigen-specific T cells is the normal consequence of antigen recognition. Expanded T cell clones have been detected in benign inflammatory skin disorders and expanded CD8⁺ T cell clones are frequently found in the peripheral circulation of older individuals and are thought to represent expanded populations of anti-CMV specific T cells (12-14). Because many benign inflammatory skin diseases are thought to be antigen driven, for example in response to allergens in atopic dermatitis and to autoantigens in psoriasis, is not surprising that predominant T cell clones have been observed in these disorders.

Indeed, the proportion of the top T cell clone, expressed as a percentage of the total T cell population, did not clearly distinguish between CTCL and benign inflammatory skin disorders (FIGS. 7D, 7E). This analysis evaluates the frequency of the top clone with respect to the remaining T cell population, but it is not a measure of the absolute number of clonal T cells in a particular unit of skin. One way of measuring the absolute number of clonal T cells in a particular skin volume is to determine what fraction of the total DNA derived from all nucleated cells in skin is contributed by the top T cell clone. This is a measure of how many clonal T cells are present in a particular unit of skin. When the data was considered in this way, we found that HTS successfully discriminated CTCL from benign inflammatory skin diseases (FIGS. 8F, 8G, 9B, 9C). In other words, particular T cell clones may be frequent within the total T cell population in benign inflammatory skin diseases but the absolute number of these cells per unit skin rarely exceeded a certain threshold (e.g., a threshold of 1:500, 1:600, 1:700, 1:800, 1:900, or preferably 1:1000 or greater). In CTCL by contrast, clonal T cells accumulated not only in frequency but also in absolute numbers to levels that exceed those observed in benign inflammatory skin diseases.

In addition to its superior ability to diagnose CTCL the early stages and to discriminate it from benign inflammatory skin diseases, the methods of the invention were also useful in following the clinical courses of established CTCL patients. By identifying and quantifying the malignant T cell clone in both blood and skin, HTS detected early disease recurrences, discriminated recurrences from benign inflammatory processes within the skin and allowed us to follow the burden of clonal malignant T cells over time (FIGS. 9A-9I). A wealth of additional information was also generated by this technique, including identification and quantification of any additional T cell clones that exist and the number and diversity of benign infiltrating T cells in skin lesions and in the blood (FIGS. 10A-10I). The presence of tumor infiltrating lymphocytes has been correlated with better outcomes in a variety of human cancers (15). HTS may therefore be a useful tool in evaluating the health of a patient's T cell repertoire perhaps also gauging their likelihood to progress.

In addition to its clinical uses, the methods of the invention are a powerful research tool that allows a more selective identification and study of malignant T cells than has been previously possible. By combining findings of TCR beta and gamma HTS, the average malignant T cell clone was calculated to contain 1.8 rearranged TCR gamma alleles, the exact proportion observed in mature peripheral blood T cells (9). This observation demonstrated that CTCL is a malignancy of mature T cells and is not a malignancy of immature T cells or lymphoid progenitor cells. HTS of the TCR β allele allowed identification of the Vβ usage of the malignant clone. With this information, malignant T cells can be identified by immunostaining with commercially available TCR Vβ antibodies. This will allow selective study of the cytokine production and gene expression by immunostainining or by laser capture micro dissection combined with RT-PCR and transcriptional profiling. It should be noted that immunostaining for the malignant clone TCR Vβ will also identify benign T cells utilizing the same TCR Vβ. In cases where the malignant T cells greatly outnumber benign T cells, this is less significant. However, in situations of low clonal predominance, in situ hybridization for the exact CDR3 nucleotide sequence of the clone could be used to selectively identify malignant T cells.

The methods of the invention provide superior sensitivity than existing TCRγ PCR methods in diagnosing CTCL and distinguishing it from benign inflammatory skin disorders. The methods of the invention facilitate the clinical follow-up in patients by allowing quantification and identification of malignant T cells in the blood and skin as well as quantifying and analyzing the diversity of remaining benign T cells that make up the patient's immune repertoire. The method described above can also be used for detecting and diagnosing B cell lymphoid neoplasms and other lymphoid proliferative disorders and distinguishing the malignant conditions from benign ones.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES CITED

1. R. Willemze et al., WHO-EORTC classification for cutaneous lymphomas 10.1182/blood-2004-09-3502. Blood 105, 3768 (May 15, 2005, 2005).
2. Y. H. Kim, H. L. Liu, S. Mraz-Gernhard, A. Varghese, R. T. Hoppe, Long-term outcome of 525 patients with mycosis fungoides and Sezary syndrome: clinical prognostic factors and risk for disease progression. Archives of dermatology 139, 857 (July 2003).
3. (© 2009 National Comprehensive Cancer Network, Inc. , 2009), vol. 2009, pp. To view the most recent and complete version of the NCCN Guidelines, go online to NCCN.org.
4. D. P. Fivenson, C. A. Hanson, B. J. Nickoloff, Localization of clonal T cells to the epidermis in cutaneous T-cell lymphoma. Journal of the American Academy of Dermatology 31, 717 (November1994).
5. J. J. van Dongen et al., Design and standardization of PCR primers and protocols for detection of clonal immunoglobulin and T-cell receptor gene recombinations in suspect lymphoproliferations: report of the BIOMED-2 Concerted Action BMH4-CT98-3936. Leukemia: official journal of the Leukemia Society of America, Leukemia Research Fund, UK 17, 2257 (December 2003).
6. R. Ponti et al., TCRgamma-chain gene rearrangement by PCR-based GeneScan: diagnostic accuracy improvement and clonal heterogeneity analysis in multiple cutaneous T-cell lymphoma samples. The Journal of investigative dermatology 128, 1030 (April 2008).
7. R. van Doorn et al., Mycosis fungoides: disease evolution and prognosis of 309 Dutch patients. Archives of dermatology 136, 504 (April 2000).
8. H. S. Robins et al., Comprehensive assessment of T-cell receptor beta-chain diversity in alphabeta T cells. Blood 114, 4099 (Nov. 5, 2009).
9. A. M. Sherwood et al., Deep sequencing of the human TCRgamma and TCRbeta repertoires suggests that TCRbeta rearranges after alphabeta and gammadelta T cell commitment. Sci Transl Med 3, 90ra61 (Jul. 6, 2011).
10. J. Marcus Muche et al., Cellular coincidence of clonal T cell receptor rearrangements and complex clonal chromosomal aberrations-a hallmark of malignancy in cutaneous T cell lymphoma. The Journal of investigative dermatology 122, 574 (March 2004).
11. H. M. Padilla-Nash, K. Wu, H. Just, T. Ried, K. Thestrup-Pedersen, Spectral karyotyping demonstrates genetically unstable skin-homing T lymphocytes in cutaneous T-cell lymphoma. Exp Dermatol 16, 98 (February 2007).
12. N. Khan et al., Cytomegalovirus seropositivity drives the CD8 T cell repertoire toward greater clonality in healthy elderly individuals. J Immunol 169, 1984 (Aug. 15, 2002).
13. W. J. Lin, D. A. Norris, M. Achziger, B. L. Kotzin, B. Tomkinson, Oligoclonal expansion of intraepidermal T cells in psoriasis skin lesions. The Journal of investigative dermatology 117, 1546 (December 2001).
14. L. I. Sakkas et al., Oligoclonal T cell expansion in the skin of patients with systemic sclerosis. J Immunol168, 3649 (Apr. 1, 2002).
15. C. Linnemann, R. Mezzadra, T. N. Schumacher, TCR repertoires of intratumoral T-cell subsets. Immunol Rev 257, 72 (January 2014).

1-15. (canceled)

16. A method for diagnosing a lymphoid malignancy in a human subject, comprising:
obtaining a genomic DNA sample from the human subject;
generating a profile of rearranged T-cell receptor (TCR) complementarity determining region-3 (CDR3) sequences, the profile comprising a frequency of each unique TCR CDR3 rearranged sequence;
identifying a T cell clone with the highest frequency of occurrence in a total number of nucleated cells in the sample; and
determining whether the T cell clone with the highest frequency of occurrence has a frequency of occurrence that is above or below a predetermined threshold for malignancy, wherein a frequency of occurrence above the predetermined threshold indicates a lymphoid malignancy in the subject.

17. The method of claim 16, further comprising diagnosing the subject with a lymphoid malignancy.

18. The method of claim 17, wherein diagnosis is at an early stage.

19. The method of claim 16, further comprising detecting a lymphoid malignancy in a subject in whom a lymphoid malignancy was not identified using another detection method.

20. The method of claim 16, wherein the predetermined threshold for malignancy is a threshold of 1 in 1000 total nucleated cells.

21. The method of claim 16, wherein the predetermined threshold for malignancy is equal to or greater than a threshold of 1 in 1000 total nucleated cells.

22. The method of claim 16, further comprising determining that the most frequent T cell clone has a frequency of occurrence that is at least one standard deviation above or below the predetermined threshold.

23. The method of claim 16, further comprising determining that the most frequent T cell clone has a frequency of occurrence that is statistically significantly different at p<0.05 from the predetermined threshold.

24. The method of claim 16, further comprising determining that the most frequent T cell clone has a frequency of occurrence that is statistically significantly different at p<0.01 from the predetermined threshold.

25. The method of claim 16, further comprising determining that the most frequent T cell clone has a frequency of occurrence that is statistically significantly different at p<0.001 from the predetermined threshold.

26. The method of claim 16, wherein the sample is obtained from a blood sample.

27. The method of claim 16, wherein generating a profile of rearranged T-cell receptor (TCR) complementarity determining region-3 (CDR3) sequences comprises:
amplifying the rearranged T-cell receptor (TCR) complementarity determining region-3 (CDR3) sequences in a single multiplex PCR using a plurality of V-segment primers and a plurality of J-segment primers to produce a plurality of amplicons representing the diversity of TCR genes in the sample; and
sequencing the plurality of amplicons to produce a plurality of sequence reads.

28. The method of claim 27, further comprising correcting for amplification bias in the plurality of V-segment primers and plurality of J-segment primers.

29. A kit for diagnosing a lymphoid malignancy in a human subject, comprising:
compositions for amplifying genomic DNA obtained from a sample from the human subject in a single multiplex PCR; and
instructions for amplification of the genomic DNA and high throughput sequencing and instructions for determining whether a top T cell clone in the sample has a frequency of occurrence that is above or below a predetermined threshold for malignancy, wherein a frequency of occurrence above the predetermined threshold is indicates a lymphoid malignancy in the subject.

30. The method of claim 16, wherein the lymphoid malignancy is selected from: acute T-cell lymphoblastic leukemia (T-ALL), acute B-cell lymphoblastic leukemia (B-ALL), multiple myeloma, plasmacytoma, macroglobulinemia, chronic lymphocytic leukemia (CLL), acute lymphoblastic leukemia (ALL), multiple myeloma, plasmacytoma, macroglobulinemia, Hodgkins lymphoma, non-Hodgkins lymphoma, cutaneous T-cell lymphoma (CTCL), mantle cell lymphoma, peripheral T-cell lymphoma, hairy cell leukemia, T prolymphocytic lymphoma, angioimmunoblastic T-cell lymphoma, T lymphoblastic leukemia/lymphoma, peripheral T-cell lymphoma, adult T cell leukemia/lymphoma, mycosis fungoides, Sezary syndrome, T lymphoblastic leukemia, myeloproliferative neoplasm, and myelodysplastic syndrome.

31-84. (canceled)

85. The method of claim 30, wherein the lymphoid malignancy is CTCL.

86. The method of claim 16, wherein the predetermined threshold is determined by:
determining a T cell clone with the highest frequency of occurrence in one or more samples from subjects with a non-malignant condition;
determining the T cell clone with the highest frequency in one or more samples obtained from subjects previously diagnosed with a lymphoid malignancy;
comparing the frequencies of occurrence of T cell clones with the highest frequency from subjects with a non-malignant condition with frequencies of occurrence of T cells clones from subjects previously diagnosed with a lymphoid malignancy and determining a threshold for malignancy based on the comparison.

87. The method of claim 86 wherein determining the T cell clone with the highest frequency of occurrence comprises determining the frequency of the most frequent T cell clone a a fraction of a total number of nucleated cells in the sample.

88. The method of claim 86 wherein determining the T cell clone with the highest frequency of occurrence in the sample comprises determining a percentage.
